(19) United States
(12) Patent Application Publication
(10) Pub. No.: US 2011/0160185 A9
(43) Pub. Date: Jun. 30, 2011

(54) PYRROLOPYRIMIDINE DERIVATIVES AS JAK3 INHIBITORS

(76) Inventors: Jorge Salas Solana, Barcelona (ES); Carmen Almansa Rosales, Barcelona (ES); Robert Soliva Soliva, Barcelona (ES); Montserrat Fontes Ustrell, Barcelona (ES); Marc Vendrell Escobar, Barcelona (ES)

(21) Appl. No.: 12/594,228

(22) PCT Filed: Mar. 31, 2008
(86) PCT No.: PCT/EP08/53842

§ 371 Date: Nov. 18, 2009

(30) Foreign Application Priority Data
Apr. 2, 2007 (EP) 07380088.0

(51) Int. Cl. A61K 31/55; C07D 487/04; A61K 31/519; A61K 31/5377; A61K 31/541; A61K 31/497; A61K 31/551

(52) U.S. Cl. 514/214.03; 544/280; 544/117; 544/58.2; 540/600; 540/575; 540/582; 514/265.1; 514/234.2; 514/228.5; 514/252.16; 514/217.06; 514/218

(57) ABSTRACT

Pyrrolopyrimidine derivatives of Formula (I) that are useful as JAK3 kinase inhibitors.

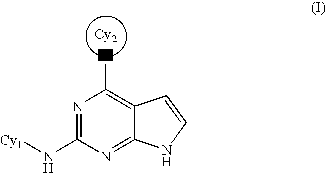

### FIELD OF THE INVENTION

The present invention relates to a new series of pyrrolopyrimidine derivatives, as well as to processes for their preparation, to pharmaceutical compositions comprising them and to their use in therapy.

### BACKGROUND OF THE INVENTION

The Janus kinases (JAKs) are cytoplasmic protein tyrosine kinases that play pivotal roles in pathways that modulate cellular functions in the lympho-hematopoietic system that are critical for cell proliferation and cell survival. JAKs are involved in the initiation of cytokine-triggered signaling events by activating through tyrosine phosphorylation the signal transducers and activators of transcription (STAT) proteins. JAK/STAT signaling has been implicated in the mediation of many abnormal immune responses such as transplant rejection and autoimmune diseases, as well as in solid and hematologic malignancies such as leukemias and lymphomas and in myeloproliferative disorders, and has thus emerged as an interesting target for drug intervention.

Four members of the JAK family have been identified so far: JAK1, JAK2, JAK3 and Tyk2. Unlike JAK1, JAK2 and Tyk2, whose expression is ubiquitous, JAK3 is mainly found in hematopoietic cells. JAK3 is associated in a non-covalent manner with the γc subunit of the receptors of IL-2, IL-4, IL-7, IL-9, IL-13 and IL-15. These cytokines play an important role in the proliferation and differentiation of T lymphocytes. JAK3-deficient mouse T cells do not respond to IL-2. This cytokine is fundamental in the regulation of T lymphocytes. In this regard, it is known that antibodies directed against the IL-2 receptor are able to prevent transplant rejection. In patients with X severe combined immunodeficiency (X-SCID), very low levels of JAK3 expression as well as genetic defects in the γc subunit of the receptor have been identified, which indicates that immunosuppression is a consequence of an alteration in the JAK3 signaling pathway.

Animal studies have suggested that JAK3 not only plays a critical role in T and B lymphocyte maturation, but also that JAK3 is required to maintain lymphocyte function. Modulation of the immunological activity through this new mechanism can prove useful in the treatment of T cell proliferative disorders such as transplant rejection and autoimmune diseases.

JAK3 has also been shown to play an important role in mast cells, because antigen-induced degranulation and mediator release have been found to be substantially reduced in mast cells from JAK3 deficient mice. JAK3 deficiency does not affect mast cell proliferation nor IgE receptor expression levels. On the other hand, JAK3−/− and JAK3+/+ mast cells contain the same intracellular mediators. Therefore, JAK3 appears to be essential in the IgE-induced release of mediators in mast cells and its inhibition would be, thus, an effective treatment for allergic reactions.

In conclusion, JAK3 kinase inhibitors have been recognised as a new class of effective immunosuppressive agents useful for transplant rejection prevention and in the treatment of immune, autoimmune, inflammatory and proliferative diseases such as psoriasis, psoriatic arthritis, rheumatoid arthritis, multiple sclerosis, inflammatory bowel diseases, systemic lupus erythematosus, type I diabetes and complications from diabetes, allergic reactions and leukemia (see e.g. O′Shea J. J. et al, Nat. Rev. Drug. Discov. 2004, 3(7):555-64; Cetkovic-Cvrlje M. et al, Curr. Pharm. Des. 2004, 10(15):1767-84; Cetkovic-Cvrlje M. et al, Arch. Immunol. Ther. Exp. (Warsz), 2004, 52(2):69-82).

Accordingly, it would be desirable to provide novel compounds that are capable of inhibiting JAK/STAT signaling pathways, and in particular which are capable of inhibiting JAK3 activity, and which are good drug candidates. Compounds should exhibit good activity in in vivo pharmacological assays, good oral absorption when administered by the oral route, as well as be metabolically stable and exhibit a favourable pharmacokinetic profile. Moreover, compounds should not be toxic and exhibit few side effects.

### DESCRIPTION OF THE INVENTION

One aspect of the invention relates to a compound of formula I

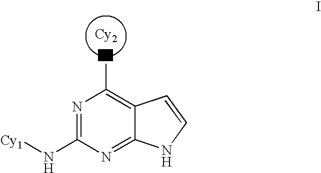

wherein:

Cy₁ represents phenyl or a 5- or 6-membered aromatic heterocycle bonded to the NH group through a C atom, each of which can be optionally fused to a 5- or 6-membered saturated, partially unsaturated or aromatic carbocyclic or heterocyclic ring, wherein Cy₁ can contain from 1 to 4 heteroatoms selected from N, O and S, wherein one or more C or S atoms of the optional 5- or 6-membered fused ring can be optionally oxidized forming CO, SO or SO₂ groups, and wherein Cy₁ can be optionally substituted with one or more R₁;

Cy₂ represents a 3- to 7-membered monocyclic or 6- to 11-membered bicyclic heterocycle, wherein the ring which contains the N atom which is bonded to the pyrrolopyrimidine moiety is saturated or partially unsaturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂;

each R₁ and R₂ independently represent C₁₋₄alkyl, C₂₋₄alkenyl, C₂₋₄alkynyl, halogen, —ON, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —NR₅SO₂R₄, —C(═N—OH)R₄ or Cy₃, wherein C₁₋₄alkyl, C₂₋₄alkenyl and C₂₋₄alkynyl can be optionally substituted with one or more R₆ and Cy₃ can be optionally substituted with one or more R₇;

R₃ represents hydrogen or R₄;

R₄ represents C₁₋₄alkyl, C₂₋₄alkenyl, C₂₋₄alkynyl, or Cy₄, wherein C₁₋₄alkyl, C₂₋₄alkenyl and C₂₋₄alkynyl can be optionally substituted with one or more R₆ and Cy₄ can be optionally substituted with one or more R₈;

R₅ represents hydrogen or C₁₋₄alkyl;

R₆ represents halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, —C(═N—OH)R₁₀ or Cy₄, wherein Cy₄ can be optionally substituted with one or more R₈;

R₇ represents C₁₋₄alkyl that can be optionally substituted with one or more R₁₁, or R₇ represents any of the meanings described for R₁₂;

R₈ represents C₁₋₄alkyl, haloC₁₋₄alkyl, C₁₋₄alkoxyCᵢ₋₄alkyl, hydroxyC₁₋₄alkyl, cyanoC₁₋₄alkyl or any of the meanings described for R₁₂;

R₉ represents hydrogen or R₁₀;

R₁₀ represents C₁₋₄alkyl, haloC₁₋₄alkyl, C₁₋₄alkoxyC₁₋₄alkyl, hydroxyC₁₋₄alkyl, cyanoC₁₋₄alkyl, Cy₅-C₁₋₄alkyl or Cy₄, wherein Cy₄ can be optionally substituted with one or more R₈;

R₁₁ represents halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, or —C(═N—OH)R₁₀;

R₁₂ represents halogen, —CN, —NO₂, —COR₁₃, —CO₂R₁₃, —CONR₁₃R₁₃, —OR₁₃, —OCOR₁₄, —OCONR₁₄R₁₄, —OCO₂R₁₄, —SR₁₃, —SOR₁₄, —SO₂R₁₄, —SO₂NR₁₃R₁₃, —SO₂NR₅COR₁₄, —NR₁₃R₁₃, —NR₅COR₁₃, —NR₅CONR₁₃R₁₃, —NR₅CO₂R₁₄, —NR₅SO₂R₁₄ or —C(═N—OH)R₁₄;

R₁₃ represents hydrogen or R₁₄;

R₁₄ represents C₁₋₄alkyl, haloC₁₋₄alkyl, C₁₋₄alkoxyC₁₋₄alkyl or hydroxyC₁₋₄alkyl;

or two R₁₃ groups or two R₁₄ groups on the same N atom can be bonded completing, together with the N atom, a 5- or 6-membered saturated ring, which can additionally contain one or two heteroatoms selected from N, S and O and which can be optionally substituted with one or more C₁₋₄alkyl groups;

each Cyₐ and Cy₄ independently represent a 3- to 7-membered monocyclic or 6- to 11-membered bicyclic ring which can be carbocyclic or heterocyclic, in which case it can contain from 1 to 4 heteroatoms selected from N, S and O, wherein each Cyₐ and Cy₄ can be saturated, partially unsaturated or aromatic, and can be bonded to the rest of the molecule through any available C or N atom, and wherein one or more C or S atoms of the ring can be optionally oxidized forming CO, SO or SO₂ groups;

Cy₅ represents a ring selected from (a)-(c):

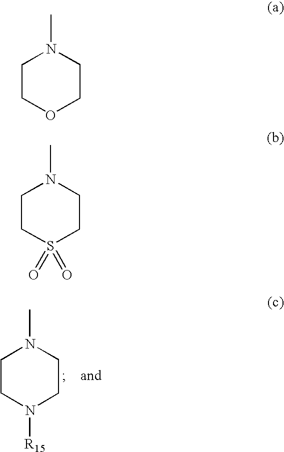

R₁₅ represents hydrogen or C₁₋₄alkyl.

The present invention also relates to the salts and solvates of the compounds of formula I.

Some compounds of formula I can have chiral centers that can give rise to various stereoisomers. The present invention relates to each of these stereoisomers and also mixtures thereof.

The compounds of formula I are JAK, particularly JAK3, kinase inhibitors and therefore can be useful for the treatment of any disease mediated by this kinase.

Thus, another aspect of the invention relates to a compound of formula I

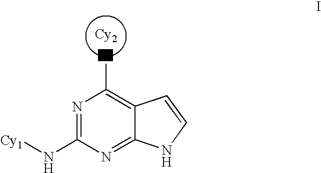

wherein:

Cy₁ represents phenyl or a 5- or 6-membered aromatic heterocycle bonded to the NH group through a C atom, each of which can be optionally fused to a 5- or 6-membered saturated, partially unsaturated or aromatic carbocyclic or heterocyclic ring, wherein Cy₁ can contain from 1 to 4 heteroatoms selected from N, O and S, wherein one or more C or S atoms of the optional 5- or 6-membered fused ring can be optionally oxidized forming CO, SO or SO₂ groups, and wherein Cy₁ can be optionally substituted with one or more R₁;

Cy₂ represents a 3- to 7-membered monocyclic or 6- to 11-membered bicyclic heterocycle, wherein the ring which contains the N atom which is bonded to the pyrrolopyrimidine moiety is saturated or partially unsaturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂;

each R₁ and R₂ independently represent C₁₋₄alkyl, C₂₋₄alkenyl, C₂₋₄alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —NR₅SO₂R₄, —C(═N—OH)R₄ or Cy₃, wherein C₁₋₄alkyl, C₂₋₄alkenyl and C₂₋₄alkynyl can be optionally substituted with one or more R₆ and Cy₃ can be optionally substituted with one or more R₇;

R₃ represents hydrogen or R₄;

R₄ represents C₁₋₄alkyl, C₂₋₄alkenyl, C₂₋₄alkynyl, or Cy₄, wherein C₁₋₄alkyl, C₂₋₄alkenyl and C₂₋₄alkynyl can be optionally substituted with one or more R₆ and Cy₄ can be optionally substituted with one or more R₈;

R₅ represents hydrogen or C₁₋₄alkyl;

R₆ represents halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, —C(═N—OH)R₁₀ or Cy₄, wherein Cy₄ can be optionally substituted with one or more R₈;

R₇ represents C₁₋₄alkyl that can be optionally substituted with one or more R₁₁, or R₇ represents any of the meanings described for R₁₂;

R₈ represents C₁₋₄alkyl, haloC₁₋₄alkyl, C₁₋₄alkoxyC₁₋₄alkyl, hydroxyC₁₋₄alkyl, cyanoC₁₋₄alkyl or any of the meanings described for R₁₂;

R₉ represents hydrogen or R₁₀;

R₁₀ represents C₁₋₄alkyl, haloC₁₋₄alkyl, C₁₋₄alkoxyC₁₋₄alkyl, hydroxyC₁₋₄alkyl, cyanoC₁₋₄alkyl, Cy₅-C₁₋₄alkyl or Cy₄, wherein Cy₄ can be optionally substituted with one or more R₈;

R₁₁ represents halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, or —C(═N—OH)R₁₀;

R₁₂ represents halogen, —CN, —NO₂, —COR₁₃, —CO₂R₁₃, —CONR₁₃R₁₃, —OR₁₃, —OCOR₁₄, —OCONR₁₄R₁₄, —OCO₂R₁₄, —SR₁₃, —SOR₁₄, —SO₂R₁₄, —SO₂NR₁₃R₁₃, —SO₂NR₅COR₁₄, —NR₁₃R₁₃, —NR₅COR₁₃, —NR₅CONR₁₃R₁₃, —NR₅CO₂R₁₄, —NR₅SO₂R₁₄ or —C(═N—OH)R₁₄;

R₁₃ represents hydrogen or R₁₄;

R₁₄ represents C₁₋₄alkyl, haloC₁₋₄alkyl, C₁₋₄alkoxyC₁₋₄alkyl or hydroxyC₁₋₄alkyl;

or two R₁₃ groups or two R₁₄ groups on the same N atom can be bonded completing, together with the N atom, a 5- or 6-membered saturated ring, which can additionally contain one or two heteroatoms selected from N, S and O and which can be optionally substituted with one or more C₁₋₄alkyl groups;

each Cy₃ and Cy₄ independently represent a 3- to 7-membered monocyclic or 6- to 11-membered bicyclic ring which can be carbocyclic or heterocyclic, in which case it can contain from 1 to 4 heteroatoms selected from N, S and O, wherein each Cy₃ and Cy₄ can be saturated, partially unsaturated or aromatic, and can be bonded to the rest of the molecule through any available C or N atom, and wherein one or more C or S atoms of the ring can be optionally oxidized forming CO, SO or SO₂ groups;

Cy₅ represents a ring selected from (a)-(c):

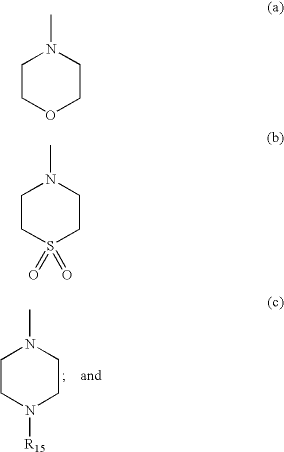

R₁₅ represents hydrogen or C₁₋₄alkyl, for use in therapy.

Another aspect of the invention relates to a pharmaceutical composition which comprises a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment of a disease mediated by JAKs, particularly JAK3.

Another aspect of the present invention relates to the use of a compound of formula I or a pharmaceutically acceptable salt thereof for the manufacture of a medicament for the treatment of at least one disease selected from transplant rejection, immune, autoimmune and inflammatory diseases, neurodegenerative diseases, and proliferative disorders. In a preferred embodiment, the disease is selected from transplant rejection and immune, autoimmune and inflammatory diseases.

Another aspect of the present invention relates to the use of a compound of formula I or a pharmaceutically acceptable salt thereof for the manufacture of a medicament for the treatment of a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas, and thromboembolic and allergic complications associated with leukemias and lymphomas.

Another aspect of the present invention relates to a compound of formula I or a pharmaceutically acceptable salt thereof for the treatment of a disease mediated by JAKs, particularly JAK3.

Another aspect of the present invention relates to a compound of formula I or a pharmaceutically acceptable salt thereof for the treatment of at least one disease selected from transplant rejection, immune, autoimmune and inflammatory diseases, neurodegenerative diseases, and proliferative disorders. In a preferred embodiment, the disease is selected from transplant rejection and immune, autoimmune and inflammatory diseases.

Another aspect of the present invention relates to a compound of formula I or a pharmaceutically acceptable salt thereof for the treatment of a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas, and thromboembolic and allergic complications associated with leukemias and lymphomas.

Another aspect of the present invention relates to the use of a compound of formula I or a pharmaceutically acceptable salt thereof for the treatment of a disease mediated by JAKs, particularly JAK3.

Another aspect of the present invention relates to the use of a compound of formula I or a pharmaceutically acceptable salt thereof for the treatment of at least one disease selected from transplant rejection, immune, autoimmune and inflammatory diseases, neurodegenerative diseases, and proliferative disorders. In a preferred embodiment, the disease is selected from transplant rejection and immune, autoimmune and inflammatory diseases.

Another aspect of the present invention relates to the use of a compound of formula I or a pharmaceutically acceptable salt thereof for the treatment of a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas, and thromboembolic and allergic complications associated with leukemias and lymphomas.

Another aspect of the present invention relates to a method of treating a disease mediated by JAKs, particularly JAK3, in a subject in need thereof, especially a human being, which comprises administering to said subject a compound of formula I or a pharmaceutically acceptable salt thereof.

Another aspect of the present invention relates to a method of treating at least one disease selected from transplant rejection, immune, autoimmune and inflammatory diseases, neurodegenerative diseases, and proliferative disorders in a subject in need thereof, especially a human being, which comprises administering to said subject a compound of formula I or a pharmaceutically acceptable salt thereof. In a preferred embodiment, the disease is selected from transplant rejection and immune, autoimmune and inflammatory diseases.

Another aspect of the present invention relates to a method of treating a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas, and thromboembolic and allergic complications associated with leukemias and lymphomas in a subject in need thereof, especially a human being, which comprises administering to said subject a compound of formula I or a pharmaceutically acceptable salt thereof.

Another aspect of the present invention relates to a process for the preparation of a compound of formula I as defined above, which comprises:

(a) reacting a compound of formula IV with a compound of formula V

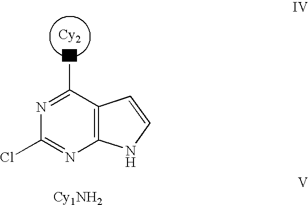

wherein Cy₁ and Cy₂ have the previously described meaning; or

(b) converting, in one or a plurality of steps, a compound of formula I into another compound of formula I.

In the above definitions, the term C₁₋₄ alkyl, as a group or part of a group, means a straight or branched alkyl chain which contains from 1 to 4 carbon atoms and includes the groups methyl, ethyl, propyl, isopropyl, butyl, isobutyl, sec-butyl and tert-butyl.

A C₂₋₄alkenyl group means a straight or branched alkyl chain which contains from 2 to 4 C atoms, and also contains one or two double bonds. Examples include the groups ethenyl, 1-propenyl, 2-propenyl, isopropenyl, 1-butenyl, 2-butenyl, 3-butenyl and 1,3-butadienyl.

A C₂₋₄alkynyl group means straight or branched alkyl chain which contains from 2 to 4 C atoms, and also contains one or two triple bonds. Examples include the groups ethynyl, 1-propynyl, 2-propynyl, 1-butynyl, 2-butynyl, 3-butynyl and 1,3-butadiynyl.

A C₁₋₄alkoxy group, as a group or part of a group, means a group of formula —OC₁₋₄alkyl, wherein the C₁₋₄alkyl moiety has the same meaning as previously described. Examples include methoxy, ethoxy, propoxy, isopropoxy, butoxy, isobutoxy, sec-butoxy and tert-butoxy.

Halogen or its abbreviation halo means fluoro, chloro, bromo or iodo.

A C₁₋₄alkoxyC₁₋₄alkyl group means a group resulting from the replacement of one or more hydrogen atoms from a C₁₋₄alkyl group with one or more C₁₋₄alkoxy groups as defined above, which can be the same or different. Examples include, among others, the groups methoxymethyl, ethoxymethyl, propoxymethyl, isopropoxymethyl, butoxymethyl, isobutoxymethyl, sec-butoxymethyl, tert-butoxymethyl, dimethoxymethyl, 1-methoxyethyl, 2-methoxyethyl, 2-ethoxyethyl, 1,2-diethoxyethyl, 1-butoxyethyl, 2-sec-butoxyethyl, 3-methoxypropyl, 2-butoxypropyl, 1-methoxy-2-ethoxypropyl, 3-tert-butoxypropyl and 4-methoxybutyl.

A haloC₁₋₄alkyl group means a group resulting from the replacement of one or more hydrogen atoms from a C₁₋₄alkyl group with one or more halogen atoms (i.e. fluoro, chloro, bromo or iodo), which can be the same or different. Examples include, among others, the groups trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl and nonafluorobutyl.

A hydroxyC₁₋₄alkyl group means a group resulting from the replacement of one or more hydrogen atoms from a C₁₋₄alkyl group with one or more hydroxy groups. Examples include, among others, the groups hydroxymethyl, 1-hydroxyethyl, 2-hydroxyethyl, 1,2-dihydroxyethyl, 3-hydroxypropyl, 2-hydroxypropyl, 1-hydroxypropyl, 2,3-dihydroxypropyl, 4-hydroxybutyl, 3-hydroxybutyl, 2-hydroxybutyl and 1-hydroxybutyl.

A cyanoC₁₋₄alkyl group means a group resulting from the replacement of one or more hydrogen atoms from a C₁₋₄alkyl group with one or more cyano groups. Examples include, among others, the groups cyanomethyl, dicyanomethyl, 1-cyanoethyl, 2-cyanoethyl, 3-cyanopropyl, 2,3-dicyanopropyl and 4-cyanobutyl.

A Cy₅-C₁₋₄alkyl group means a group resulting from the replacement of one hydrogen atom from a C₁₋₄alkyl group with one Cy₅ group. Examples include, among others, the groups (morpholin-4-yl)methyl, 2-(morpholin-4-yl)ethyl, 3-(morpholin-4-yl)propyl, 4-(morpholin-4-yl)butyl, (piperazin-1-yl)methyl, (4-methylpiperazin-1-yl)methyl, 2-(4-methylpiperazin-1-yl)ethyl, 3-(4-methylpiperazin-1-yl)propyl, 4-(4-methylpiperazin-1-yl)butyl, (4-ethyl piperazin-1-yl)methyl, (4-propylpiperazin-1-yl)methyl, (4-butylpiperazin-1-yl)methyl, (1,1-dioxothiomorpholin-4-yl)methyl, 2-(1,1-dioxothiomorpholin-4-yl)ethyl, 3-(1,1-dioxothiomorpholin-4-yl)propyl and 4-(1,1-dioxothiomorpholin-4-yl)butyl.

A Cy₄-C₁₄alkyl group means a group resulting from the replacement of one hydrogen atom from a C₁₋₄alkyl group with one Cy₄ group as defined above.

A group NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, R₉CO—C₁₋₄alkyl, NR₉R₉—C₁₋₄alkyl, R₁₀SO₂NR₅—C₁₋₄alkyl or NR₉R₉CONR₅—C₁₋₄alkyl means a group resulting from the replacement of one hydrogen atom from a C₁₋₄alkyl group with one —SO₂NR₉R₉, —CONR₉R₉, —SO₂NR₅COR₁₀, —NR₅COR₉, —COR₉, —NR₉R₉, —NR₅SO₂R₁₀ or —NR₅CONR₉R₉ group, respectively. For example, examples of a group NR₉R₉SO₂—C₁₋₄alkyl include, among others, the groups sulfamoylmethyl, 1-sulfamoylethyl, 2-sulfamoylethyl, 1-sulfamoylpropyl, 2-sulfamoylpropyl, 3-sulfamoylpropyl, 1-sulfamoylbutyl, 2-sulfamoylbutyl, 3-sulfamoylbutyl, 4-sulfamoylbutyl, N-methylsulfamoylmethyl, N,N-dimethylsulfamoylmethyl and N-ethyl-N-methylsulfamoylmethyl.

The term Cy₁ refers to a phenyl group or a 5- or 6-membered aromatic heterocycle that must be bonded to the NH group through a C atom, wherein both the phenyl group and the 5- or 6-membered aromatic heterocycle can be optionally fused to a 5- or 6-membered carbocycle or heterocycle which can be saturated, partially unsaturated or aromatic. The Cy₁ group, as a whole, can contain from 1 to 4 heteroatoms in total selected from N, O and S. When the second ring, i.e. the optional 5- or 6-membered carbocyclic or heterocyclic fused ring, is saturated or partially unsaturated, one or more C or S atoms of said ring can be optionally oxidized forming CO, SO or SO₂ groups. The Cy₁ group can be optionally substituted as disclosed above in the definition of a compound of formula I; said substituents can be the same or different and can be placed on any available position of any of the rings. Examples of Cy₁ groups include, among others, phenyl, naphthyl, thienyl, furyl, pyrrolyl, thiazolyl, isothiazolyl, oxazolyl, isoxazolyl, imidazolyl, pyrazolyl, 1,2,3-triazolyl, 1,2,4-triazolyl, tetrazolyl, 1,3,4-oxadiazolyl, 1,3,4-thiadiazolyl, 1,2,4-oxadiazolyl, 1,2,4-thiadiazolyl, pyridyl, pyrazinyl, pyrimidinyl, pyridazinyl, benzimidazolyl, benzooxazolyl, benzofuranyl, isobenzofuranyl, indolyl, isoindolyl, benzothiophenyl, benzothiazolyl, quinolinyl, isoquinolinyl, phtalazinyl, quinazolinyl, quinoxalinyl, cinolinyl, naphthyridinyl, indazolyl, imidazopyridinyl, pyrrolopyridinyl, thienopyridinyl, imidazopyrimidinyl, imidazopyrazinylz, imidazopyridazinyl, pyrazolopyrazinyl, pyrazolopyridinyl, pyrazolopyrimidinyl, benzo[1,3]dioxolyl, phtalimidyl, 1-oxo-1,3-dihydroisobenzofuranyl, 1,3-dioxo-1,3-dihydroisobenzofuranyl, 2-oxo-2,3-dihydro-1H-indolyl, 1-oxo-2,3-dihydro-1H-isoindolyl, 1,2,3,4-tetrahydroquinolinyl, 1,2,3,4-tetrahydroisoquinolinyl, 1-oxo-1,2,3,4-tetrahydroisoquinolinyl, 1-oxo-1,2-dihydroisoquinolinyl and 4-oxo-3,4-dihydroquinazolinyl.

The term Cy₂ refers to a 3- to 7-membered monocyclic or a 6- to 11-membered bicyclic heterocycle, with the proviso that the ring directly bonded to the pyrrolopyrimidine is saturated or partially unsaturated. When Cy₂ is bicyclic, the second ring can be saturated, partially unsaturated or aromatic. Cy₂ contains from 1 to 4 heteroatoms in total selected from N, O and S including the N atom bonding Cy₂ to the pyrrolopyrimidine ring, so that Cy₂ always contains at least one N atom. When Cy₂ is a bicyclic ring, this can be formed by two rings fused through two adjacent C or N atoms, or through two non-adjacent C or N atoms forming a bridged ring, or else it can be formed by two rings sharing a C atom as a single common atom thus forming a spiro ring. In Cy₂ one or more C or S atoms in any saturated or partially unsaturated ring can be optionally oxidized forming CO, SO or SO₂ groups. The Cy₂ group can be optionally substituted as disclosed above in the definition of a compound of formula I; said substituents can be the same or different and can be placed on any available position of the ring system. Examples of Cy₂ groups include, among others, azepanyl, aziridinyl, azetidinyl, 1,4-diazepanyl, pyrrolidinyl, imidazolidinyl, isoxazolidinyl, oxazolidinyl, pyrazolidinyl, thiazolidinyl, isothiazolidinyl, imidazolinyl, pyrrolinyl, pyrazolinyl, piperidinyl, homopiperidinyl, morpholinyl, thiomorpholinyl, 1,1-dioxothiomorpholinyl, piperazinyl, homopiperazinyl, 2-oxo-azepanyl, 2-oxo-azetidinyl, 2-oxo-1,4-diazepanyl, 2-oxo-pyrrolidinyl, 2-oxo-piperazinyl, 2-oxo-piperidinyl, 3-oxo-piperidinyl, 4-oxo-piperidinyl, 2-oxo-imidazolidinyl, 2-oxo-oxazolidinyl, 2-oxo-1,2-dihydropyridinyl, 2-oxo-1,2-dihydropyrazinyl, 2-oxo-1,2-dihydropyrimidinyl, 3-oxo-2,3-dihydropyridazinyl, 1,2,3,6-tetrahydropyridinyl, perhydroisoquinolinyl, 1-oxo-1,2-dihydroisoquinolinyl, 4-oxo-3,4-dihydroquinazolinyl, 5-aza-bicyclo[2.1.1]hexanyl, 2-aza-bicyclo[2.2.1]heptanyl, 6-aza-bicyclo[3.2.1]octanyl, octahydro-pyrrolo[1,2-a]pyrazinyl, 2H-spiro[benzofuran-3,4′-piperidinyl], 3H-spiro[isobenzofuran-1,4′-piperidinyl], 2,8-diazaspiro[4.5]decan-1-onyl, 2,7-diazaspiro[4.5]decan-1-onyl, 2-aza-bicyclo[2.2.1]heptan-6-onyl and 6-aza-bicyclo[3.2.1]octan-7-onyl.

The term Cy₃ or Cy₄ refers to a 3- to 7-membered monocyclic or 6- to 11-membered bicyclic carbocyclic or heterocyclic ring. When heterocyclic, it can contain from 1 to 4 heteroatoms selected from N, S and O. Bicyclic rings may be formed either by two rings fused through two adjacent C or N atoms, or through two non-adjacent C or N atoms forming a bridged ring, or else they can be formed by two rings bonded through a single common C atom forming a spiro ring. A Cy₃ or Cy₄ group can be saturated, partially unsaturated or aromatic. Cy₃ and Cy₄ can be bonded to the rest of the molecule through any available C or N atom. In Cy₃ or Cy₄ one or more C or S atoms of a saturated or partially unsaturated ring can be optionally oxidized forming CO, SO or SO₂ groups. Cy₃ and Cy₄ can be optionally substituted as disclosed above in the definition of a compound of formula I; if substituted, said substituents can be the same or different and can be placed on any available position of the ring system. Examples of Cy₃ or Cy₄ groups include, among others, cyclopropyl, cyclobutyl, cyclopentyl, cyclohexyl, cycloheptyl, azetidinyl, aziridinyl, oxiranyl, oxetanyl, imidazolidinyl, isothiazolidinyl, isoxazolidinyl, oxazolidinyl, pyrazolidinyl, pyrrolidinyl, thiazolidinyl, dioxanyl, morpholinyl, thiomorpholinyl, 1,1-dioxothiomorpholinyl, piperazinyl, homopiperazinyl, piperidinyl, pyranyl, tetrahydropyranyl, homopiperidinyl, oxazinyl, oxazolinyl, pyrrolinyl, thiazolinyl, pyrazolinyl, imidazolinyl, isoxazolinyl, isothiazolinyl, 2-oxo-pyrrolidinyl, 2-oxo-piperidinyl, 4-oxo-piperidinyl, 2-oxo-piperazinyl, 2-oxo-1,2-dihydropyridinyl, 2-oxo-1,2-dihydropyrazinyl, 2-oxo-1,2-dihydropyrimidinyl, 3-oxo-2,3-dihydropyridazyl, phenyl, naphthyl, thienyl, furyl, pyrrolyl, thiazolyl, isothiazolyl, oxazolyl, isoxazolyl, imidazolyl, pyrazolyl, 1,2,3-triazolyl, 1,2,4-triazolyl, tetrazolyl, 1,3,4-oxadiazolyl, 1,3,4-thiadiazolyl, 1,2,4-oxadiazolyl, 1,2,4-thiadiazolyl, pyridyl, pyrazinyl, pyrimidinyl, pyridazinyl, benzimidazolyl, benzooxazolyl, benzofuranyl, isobenzofuranyl, indolyl, isoindolyl, benzothiophenyl, benzothiazolyl, quinolinyl, isoquinolinyl, phtalazinyl, quinazolinyl, quinoxalinyl, cinolinyl, naphthyridinyl, indazolyl, imidazopyridinyl, pyrrolopyridinyl, thienopyridinyl, imidazopyrimidinyl, imidazopyrazinyl, imidazopyridazinyl, pyrazolopyrazinyl, pyrazolopyridinyl, pyrazolopyrimidinyl, benzo[1,3]dioxolyl, phtalimidyl, 1-oxo-1,3-dihydroisobenzofuranyl, 1,3-dioxo-1,3-dihydroisobenzofuranyl, 2-oxo-2,3-dihydro-1H-indolyl, 1-oxo-2,3-dihydro-1H-isoindolyl, perhydroquinolinyl, 1-oxo-perhydroisoquinolinyl, 1-oxo-1,2-dihydroisoquinolinyl, 4-oxo-3,4-dihydroquinazolinyl, 2-aza-bicyclo[2.2.1]heptanyl, 5-aza-bicyclo[2.1.1]hexanyl, 2H-spiro[benzofuran-3,4′-piperidinyl], 3H-spiro[isobenzofuran-1,4′-piperidinyl], 2,8-diazaspiro[4.5]decan-1-onyl and 2,7-diazaspiro[4.5]decan-1-onyl.

In the above definitions of Cy₁, Cy₂, Cy₃ and Cy₄, when the examples listed refer to a bicycle in general terms, all possible dispositions of the atoms are included. Thus, for example, the term pyrazolopyridinyl can include groups such as 1H-pyrazolo[3,4-b]pyridinyl, 1H-pyrazolo[1,5-a]pyridinyl, 1H-pyrazolo[3,4-c]pyridinyl, 1H-pyrazolo[4,3-c]pyridinyl and 1H-pyrazolo[4,3-b]pyridinyl, the term imidazopyrazinyl can include groups such as 1H-imidazo[4,5-b]pyrazinyl, imidazo[1,2-a]pyrazinyl and imidazo[1,5-a]pyrazinyl and the term pyrazolopyrimidinyl can include groups such as 1H-pyrazolo[3,4-d]pyrimidinyl, 1H-pyrazolo[4,3-d]pyrimidinyl, pyrazolo[1,5-a]pyrimidinyl and pyrazolo[1,5-c]pyrimidinyl.

When in the definitions used throughout the present specification for cyclic groups the examples given refer to a radical of a ring in general terms, for example pyridyl, thienyl or indolyl, all the available bonding positions are included, unless a limitation is indicated in the corresponding definition for said cyclic group, for example that the ring is bonded through a C atom in Cy₁ or through a N atom in Cy₂, in which case such limitation applies. Thus for example, in the definitions of Cy₃ and Cy₄, which do not include any limitation regarding the bonding position, the term pyridyl includes 2-pyridyl, 3-pyridyl and 4-pyridyl; thienyl includes 2-thienyl and 3-thienyl; and indolyl includes 1-indolyl, 2-indolyl, 3-indolyl, 4-indolyl, 5-indolyl, 6-indolyl and 7-indolyl.

The expression “optionally substituted with one or more” means that a group can be substituted with one or more, preferably with 1, 2, 3 or 4 substituents, more preferably with 1, 2 or 3 substituents, and still more preferably with 1 or 2 substituents, provided that said group has enough positions susceptible of being substituted. The substituents can be the same or different and can be placed on any available position.

When a non-aromatic ring is present as a substituent of a non-aromatic ring, it can replace one hydrogen atom, or it can replace two hydrogen atoms on the same C atom thus forming a spiro ring. Likewise, when a non-aromatic ring is present as a substituent of an alkyl, alkenyl or alkynyl group, it can either replace one hydrogen atom, or it can replace two hydrogen atoms on the same C atom.

When in the definition of a substituent two or more groups with the same numbering are indicated (e.g. —NR₅CONR₃R₃, —NR₉R₉, —CONR₁₃R₁₃, etc.), this does not mean that they must be the same. Each of them is independently selected from the list of possible meanings given for said group, and therefore they can be the same or different.

In certain embodiments of the invention, Cy₂ represents a phenyl group substituted at one or two of positions 3, 4 and 5 with a R₁ group. This means that the phenyl group is either substituted with one R₁ group at position 3, 4 or 5 of the phenyl ring, or with two R₁ groups (which can be the same or different) at positions 3 and 4, positions 4 and 5 or positions 3 and 5 of the phenyl ring.

Throughout the present specification, the expressions “treatment” of a disease, “treating” a disease and the like refer both to curative treatment as well as palliative treatment or prophylactic treatment of said disease. For purposes of this invention, beneficial or desired clinical results include, but are not limited to, alleviation or amelioration of one or more symptoms, diminishment of extent of disease, stabilized (i.e. not worsening) state of disease, preventing the disease from occurring in a patient that is predisposed or does not yet display symptoms of the disease, delay or slowing of disease progression, amelioration or palliation of the disease state, and remission (whether partial or total). Those in need of treatment include those already with the disease or disorder as well as those prone to have the disease or disorder or those in which the disease or disorder is to be prevented.

The invention thus relates to the compounds of formula I as defined above.

In another embodiment, the invention relates to the compounds of formula I wherein Cy₁ represents phenyl or pyridyl, which can be optionally fused to a 5- or 6-membered saturated, partially unsaturated or aromatic carbocyclic or heterocyclic ring, wherein Cy₁ can contain from 1 to 4 heteroatoms selected from N, O and S, wherein one or more C or S atoms of the 5- or 6-membered fused ring can be optionally oxidized forming CO, SO or SO₂ groups, and wherein Cy₁ can be optionally substituted with one or more R₁.

In another embodiment, the invention relates to the compounds of formula I wherein Cy₁ represents phenyl, pyridyl or a ring of formula Cy₁ₐ,

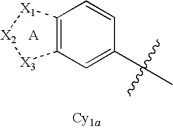

wherein in ring A X₁, X₂ and X₃ are selected from C, N, O and S and the dashed lines represent single or double bonds, wherein one or two C or S atoms of ring A can be optionally oxidized forming CO, SO or SO₂ groups, and wherein the phenyl, pyridyl and Cy₁ₐ groups can be optionally substituted with one or more R₁.

In another embodiment, the invention relates to the compounds of formula I wherein Cy₁ represents phenyl, 3-pyridyl, 4-pyridyl or a ring of formula Cy₁ₐ, each of which can be optionally substituted with one or more R₁.

In another embodiment, the invention relates to the compounds of formula I wherein Cy₁ represents phenyl, pyridyl, benzo[1,3]dioxolyl or benzooxazolyl, each of which can be optionally substituted with one or more R₁.

In another embodiment, the invention relates to the compounds of formula I wherein Cy₁ represents phenyl, 3-pyridyl, 4-pyridyl, 5-benzo[1,3]dioxolyl or 6-benzooxazolyl, which can be optionally substituted with one or more R₁.

In another embodiment, the invention relates to the compounds of formula wherein Cy₁ represents phenyl optionally substituted with one or more R₁.

In another embodiment, the invention relates to the compounds of formula wherein Cy₁ represents phenyl substituted with one or more R₁.

In another embodiment, the invention relates to the compounds of formula wherein Cy₁ represents phenyl substituted with one, two or three R₁.

In another embodiment, the invention relates to the compounds of formula wherein Cy₁ represents phenyl substituted with one or two R₁.

In another embodiment, the invention relates to the compounds of formula wherein Cy₁ represents phenyl substituted at one or two of positions 3, 4 and 5 with an R₁.

In another embodiment, the invention relates to the compounds of formula I wherein Cy₁ represents phenyl substituted with one R₁, which is placed at position 3 or 4 of the phenyl ring.

In another embodiment, the invention relates to the compounds of formula I wherein each R₁ represents C₁₋₄alkyl, C₂₋₄alkenyl, C₂₋₄alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —C(═N—OH)R₄ or Cy₃, wherein C₁₋₄alkyl, C₂₋₄alkenyl and C₂₋₄alkynyl can be optionally substituted with one or more R₆ and Cy₃ can be optionally substituted with one or more R₇.

In another embodiment, the invention relates to the compounds of formula I wherein each R₁ represents C₁₋₄alkyl, halogen, —CN, —COR₃, —CO₂R₃, —CONR₃R₃, —COCONR₃R₃, —OR₃, —SR₃, —SO₂R₄, —SO₂NR₃R₃, —SO₂NR₅COR₄, —NR₃R₃, —NR₅COR₃, —NR₅CONR₃R₃, —NR₅SO₂R₄ or Cy₃, wherein the C₁₋₄alkyl group can be optionally substituted with one or more R₆ and Cy₃ can be optionally substituted with one or more R₇.

In another embodiment, the invention relates to the compounds of formula I wherein each R₁ represents C₁₋₄alkyl, halogen, —CN, —COR₃, —CO₂R₃, —CONR₃R₃, —COCONR₃R₃, —OR₃, —SR₃, —SO₂R₄, —SO₂NR₃R₃, —SO₂NR₅COR₄, —NR₃R₃, —NR₅COR₃, —NR₅CONR₃R₃ or Cy₃, wherein the C₁₋₄alkyl group can be optionally substituted with one or more R₆ and Cy₃ can be optionally substituted with one or more R₇.

In another embodiment, the invention relates to the compounds of formula I wherein each R₁ represents C₁₋₄alkyl, halogen, —CN, —OR₃, —SO₂R₄, —SO₂NR₃R₃, —SO₂NR₅COR₄, —NR₃R₃, —NR₅COR₃, —NR₅SO₂R₄ or Cy₃, wherein the C₁₋₄alkyl group can be optionally substituted with one or more R₆ and Cy₃ can be optionally substituted with one or more R₇.

In another embodiment, the invention relates to the compounds of formula I wherein each R₁ represents C₁₋₄alkyl, halogen, haloC₁₋₄alkyl, hydroxyC₁₋₄alkyl, C₁₋₄alkoxyC₁₋₄alkyl, —CN, —OR₃, —SO₂R₄, —SO₂NR₃R₃, —SO₂NR₅COR₄, —NR₃R₃, —NR₅COR₃, —NR₅SO₂R₄ or Cy₃, wherein Cy₃ can be optionally substituted with one or more R₇.

In another embodiment, the invention relates to the compounds of formula I wherein Cy₃ in R₁ represents Cy₃ₐ, and Cy₃ₐ represents a 5- or 6-membered saturated monocyclic heterocycle which contains 1 or 2 heteroatoms selected from N, S and O, wherein said ring can be bonded to the rest of the molecule through any available C or N atom, and wherein one or more C or S atoms of the ring can be optionally oxidized forming CO, SO or SO₂ groups, wherein said Cy₃ₐ can be optionally substituted with one or more R₇.

In another embodiment, the invention relates to the compounds of formula I wherein Cy₃ in R₁ represents Cy_{3b}, and Cy_{3b} represents a 5- or 6-membered saturated monocyclic heterocycle which contains 1 or 2 heteroatoms selected from N, S and O with the proviso that it contains at least 1 N atom, wherein said ring is bonded to the rest of the molecule through a N atom, wherein one or more C or S ring atoms can be optionally oxidized forming CO, SO or SO₂ groups, and wherein said Cy_{3b} can be optionally substituted with one or more R₇.

In another embodiment, the invention relates to the compounds of formula I wherein Cy₄ in R₁ represents Cy₄ₐ, and Cy₄ₐ represents a 5- or 6-membered saturated monocyclic heterocycle which contains 1 or 2 heteroatoms selected from N, S and O and which can be bonded to the rest of the molecule through any available C or N atom, wherein one or more C or S ring atoms can be optionally oxidized forming CO, SO or SO₂ groups, and wherein said Cy₄, can be optionally substituted with one or more R₈.

In another embodiment, the invention relates to the compounds of formula I wherein each R₁ represents C₁₋₄alkyl, halogen, —CONR₃R₃, —OR₃, —SO₂NR₃R₃, —SO₂NR₅COR₄, —NR₅COR₃ or Cy₃, wherein the C₁₋₄alkyl group can be optionally substituted with one or more R₆ and Cy₃ can be optionally substituted with one or more R₇.

In another embodiment, the invention relates to the compounds of formula I wherein each R₁ represents C₁₋₄alkyl, halogen, —CONR₃R₃, —OR₃, —SO₂NR₃R₃, —SO₂NR₅COR₄, —NR₅COR₃ or Cy₃ₐ, wherein the C₁₋₄alkyl group can be optionally substituted with one or more R₆ and Cy₃ₐ can be optionally substituted with one or more R₇.

In another embodiment, the invention relates to the compounds of formula I wherein each R₁ represents C₁₋₄alkyl, halogen, —CONR₃R₃, —OR₃, —SO₂NR₃R₃, —SO₂NR₅COR₄, —NR₅COR₃ or Cy_{3b}, wherein the C₁₋₄alkyl group can be optionally substituted with one or more R₆ and Cy_{3b} can be optionally substituted with one or more R₇.

In another embodiment, the invention relates to the compounds of formula I wherein each R₁ represents C₁₋₄alkyl, hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈.

In another embodiment, the invention relates to the compounds of formula I wherein each R₁ represents C₁₋₄alkyl, hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀ CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃ₐ, wherein Cy₃ₐ can be optionally substituted with one or more R₇ and wherein Cy₄ can be optionally substituted with one or more R₈.

In another embodiment, the invention relates to the compounds of formula I wherein each R₁ represents C₁₋₄alkyl, hydroxyC₁₋₄alkyl, C₁₋₄alkoxyC₁₋₄alkyl, Cy₄, —C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃ₐ, wherein Cy₃, can be optionally substituted with one or more R₇ and wherein Cy₄, can be optionally substituted with one or more R₈.

In another embodiment, the invention relates to the compounds of formula I wherein each R₁ represents C₁₋₄alkyl, hydroxyC₁₋₄alkyl, C₁₋₄alkoxyC₁₋₄alkyl, Cy₄, —C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy_{3b}, wherein Cy_{3b} can be optionally substituted with one or more R₇ and wherein Cy₄ₐ can be optionally substituted with one or more R₈.

In another embodiment, the invention relates to the compounds of formula I wherein each R₁ represents hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈.

In another embodiment, the invention relates to the compounds of formula I wherein each R₁ represents hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, —CONR₃R₃, —OR₃, —SO₂NR₃R₃, —SO₂NR₅COR₄, —NR₅COR₃ or Cy₃ₐ, wherein Cy₃, can be optionally substituted with one or more R₇ and wherein Cy₄ can be optionally substituted with one or more R₈.

In another embodiment, the invention relates to the compounds of formula I wherein each R₁ represents hydroxyC₁₋₄alkyl, C₁₋₄alkoxyC₁₋₄alkyl, Cy₄ₐ-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, —CONR₃R₃, —OR₃, —SO₂NR₃R₃, —SO₂NR₅COR₄, —NR₅COR₃ or Cy₃ₐ, wherein Cy₃ₐ can be optionally substituted with one or more R₇ and wherein Cy₄, can be optionally substituted with one or more R₈.

In another embodiment, the invention relates to the compounds of formula I wherein each R₁ represents hydroxyC₁₋₄alkyl, C₁₋₄alkoxyC₁₋₄alkyl, Cy₄ₐ-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, —CONR₃R₃, —OR₃, —SO₂NR₃R₃, —SO₂NR₅COR₄, —NR₅COR₃ or Cy_{3b}, wherein Cy_{3b} can be optionally substituted with one or more R₇ and wherein Cy₄, can be optionally substituted with one or more R₈.

In another embodiment, the invention relates to the compounds of formula I wherein each R₁ represents C₁₋₄alkyl, halogen, haloC₁₋₄alkyl, hydroxyC₁₋₄alkyl, C₁₋₄alkoxyC₁₋₄alkyl, —CN, —OR₃, —SO₂R₄, —SO₂NR₃R₃, —SO₂NR₅COR₄, —NR₃R₃, —NR₅COR₃, —NR₅SO₂R₄ or Cy₃ₐ, wherein Cy₃, can be optionally substituted with one or more R₇.

In another embodiment, the invention relates to the compounds of formula I wherein R₃ in R₁ represents hydrogen or R₄ and R₄ in R₁ represents C₁₋₄alkyl or Cy₄, wherein C₁₋₄alkyl can be optionally substituted with one or more R₆ and wherein Cy₄ can be optionally substituted with one or more R₈.

In another embodiment, the invention relates to the compounds of formula I wherein R₃ in R₁ represents hydrogen or R₄ and R₄ in R₁ represents C₁₋₄alkyl, Cy₄-C₁₋₄alkyl, hydroxyC₁₋₄alkyl, C₁₋₄alkoxyC₁₋₄alkyl or Cy₄, wherein any Cy₄ can be optionally substituted with one or more R₈.

In another embodiment, the invention relates to the compounds of formula I wherein:

Cy₁ represents phenyl substituted with one or more R₁, preferably one or two R₁; and

each R₁ represents C₁₋₄alkyl, C₂₋₄alkenyl, C₂₋₄alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —C(═N—OH)R₄ or Cy₃, wherein C₁₋₄alkyl, C₂₋₄alkenyl and C₂₋₄alkynyl can be optionally substituted with one or more R₆ and Cy₃ can be optionally substituted with one or more R₇.

In another embodiment, the invention relates to the compounds of formula I wherein:

Cy₁ represents phenyl substituted with one or more R₁, preferably one or two R₁; and

each R₁ represents C₁₋₄alkyl, halogen, —CN, —COR₃, —CO₂R₃, —CONR₃R₃, —COCONR₃R₃, —OR₃, —SR₃, —SO₂R₄, —SO₂NR₃R₃, —SO₂NR₅COR₄, —NR₃R₃, —NR₅COR₃, —NR₅CONR₃R₃, —NR₅SO₂R₄ or Cy₃, wherein the C₁₋₄alkyl group can be optionally substituted with one or more R₆ and Cy₃ can be optionally substituted with one or more R₇.

In another embodiment, the invention relates to the compounds of formula I wherein:

Cy₁ represents phenyl substituted with one or more R₁, preferably one or two R₁; and

each R₁ represents C₁₋₄alkyl, halogen, —CONR₃R₃, —OR₃, —SO₂NR₃R₃, —SO₂NR₅COR₄, —NR₅COR₃ or Cy₃, wherein the C₁₋₄alkyl group can be optionally substituted with one or more R₆ and Cy₃ can be optionally substituted with one or more R₇.

In another embodiment, the invention relates to the compounds of formula I wherein:

Cy₁ represents phenyl substituted with one or more R₁, preferably one or two R₁; and

each R₁ represents C₁₋₄alkyl, halogen, —CONR₃R₃, —OR₃, —SO₂NR₃R₃, —SO₂NR₅COR₄, —NR₅COR₃ or Cy₃ₐ, wherein the C₁₋₄alkyl group can be optionally substituted with one or more R₆ and Cy₃ₐ can be optionally substituted with one or more R₇.

In another embodiment, the invention relates to the compounds of formula I wherein:

Cy₁ represents phenyl substituted with one or more R₁, preferably one or two R₁; and

each R₁ represents C₁₋₄alkyl, hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈.

In another embodiment, the invention relates to the compounds of formula I wherein:

Cy₁ represents phenyl substituted with one or more R₁, preferably one or two R₁; and

each R₁ represents C₁₋₄alkyl, halogen, haloC₁₋₄alkyl, hydroxyC₁₋₄alkyl, C₁₋₄alkoxyC₁₋₄alkyl, —CN, —OR₃, —SO₂R₄, —SO₂NR₃R₃, —SO₂NR₅COR₄, —NR₃R₃, —NR₅COR₃, —NR₅SO₂R₄ or Cy₃ₐ, wherein Cy₃ₐ can be optionally substituted with one or more R₇.

In another embodiment, the invention relates to the compounds of formula I wherein:

Cy₁ represents a ring of formula Cy_{1b}:

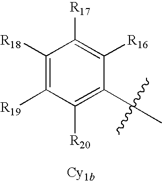

one of R₁₇, R₁₈ or R₁₉ represents hydroxyC₁₋₄alkyl, —CN, —OR₃, —SO₂R₄, —SO₂NR₃R₃, —NR₅COR₃, —NR₅SO₂R₄ or Cy₃ₐ, wherein Cy₃ₐ can be optionally substituted with one or more R₇; and

the remainder of R₁₇, R₁₈ and R₁₉ as well as R₁₆ and R₂₀ are selected from hydrogen, C₁₋₄alkyl, halogen and C₁₋₄alkoxy.

In another embodiment, the invention relates to the compounds of formula I wherein:

Cy₁ represents phenyl substituted at one or two of positions 3, 4 and 5 with an R₁; and

each R₁ represents C₁₋₄alkyl, C₂₋₄alkenyl, C₂₋₄alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —C(═N—OH)R₄ or Cy₃, wherein C₁₋₄alkyl, C₂₋₄alkenyl and C₂₋₄alkynyl can be optionally substituted with one or more R₆ and Cy₃ can be optionally substituted with one or more R₇.

In another embodiment, the invention relates to the compounds of formula I wherein:

Cy₁ represents phenyl substituted at one or two of positions 3, 4 and 5 with an R₁; and

each R₁ represents C₁₋₄alkyl, halogen, —CONR₃R₃, —OR₃, —SO₂NR₃R₃, —SO₂NR₅COR₄, —NR₅COR₃ or Cy₃, wherein the C₁₋₄alkyl group can be optionally substituted with one or more R₆ and Cy₃ can be optionally substituted with one or more R₇.

In another embodiment, the invention relates to the compounds of formula I wherein:

Cy₁ represents phenyl substituted at one or two of positions 3, 4 and 5 with an R₁; and

each R₁ represents C₁₋₄alkyl, hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈.

In another embodiment, the invention relates to the compounds of formula I wherein:

Cy₁ represents phenyl substituted with one R₁, which is placed at position 3 or 4 of the phenyl ring; and

R₁ represents C₁₋₄alkyl, hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈.

In another embodiment, the invention relates to the compounds of formula I wherein:

Cy₁ represents phenyl substituted with one R₁, which is placed at position 3 or 4 of the phenyl ring; and

R₁ represents hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈.

In another embodiment, the invention relates to the compounds of formula I wherein:

Cy₁ represents phenyl substituted with one R₁, which is placed at position 3 or 4 of the phenyl ring; and

R₁ represents hydroxyC₁₋₄alkyl, C₁₋₄alkoxyC₁₋₄alkyl, Cy₄, —C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, —CONR₃R₃, —OR₃, —SO₂NR₃R₃, —SO₂NR₅COR₄, —NR₅COR₃ or Cy_{3b}, wherein Cy_{3b} can be optionally substituted with one or more R₇ and wherein Cy₄, can be optionally substituted with one or more R₈.

In another embodiment, the invention relates to the compounds of formula I wherein:

Cy₁ represents phenyl substituted with one R₁, which is placed at position 3 or 4 of the phenyl ring;

R₁ represents hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₉—C₁₋₄alkyl, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈;

R₃ in R₁ represents hydrogen or R₄; and

R₄ in R₁ represents C₁₋₄alkyl or Cy₄, wherein C₁₋₄alkyl can be optionally substituted with one or more R₆ and wherein Cy₄ can be optionally substituted with one or more R₈.

In another embodiment, the invention relates to the compounds of formula I wherein Cy₂ represents a 5- to 7-membered monocyclic or 6- to 11-membered bicyclic heterocycle, wherein the ring which contains the N atom which is bonded to the pyrrolopyrimidine is saturated or partially unsaturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein Cy₂ represents a 5- to 7-membered monocyclic or 6- to 11-membered bicyclic heterocycle, wherein the ring which contains the N atom which is bonded to the pyrrolopyrimidine moiety is saturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein Cy₂ represents a saturated 5- to 7-membered monocyclic heterocycle which contains from 1 to 2 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein Cy₂ is selected from (a)-(i):

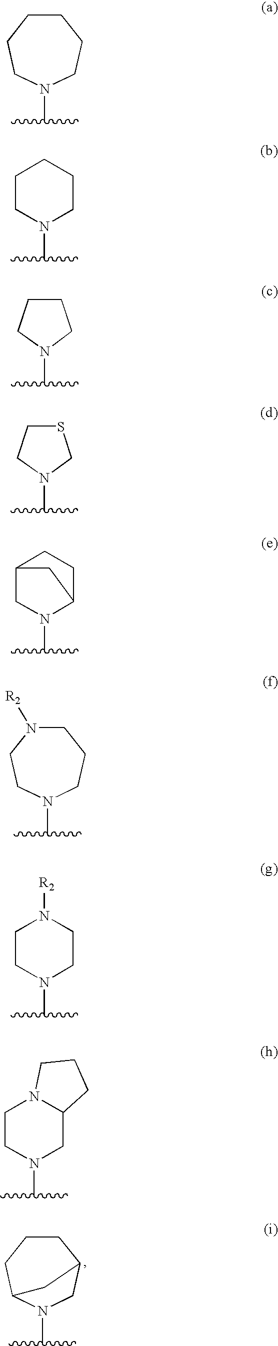

wherein one or more C or S atoms of Cy₂ can be optionally oxidized forming CO, SO or SO₂ groups, and wherein Cy₂ can be optionally substituted with one or more R₂.

In another embodiment, the invention relates to the compounds of formula I wherein Cy₂ is selected from (a)-(g):

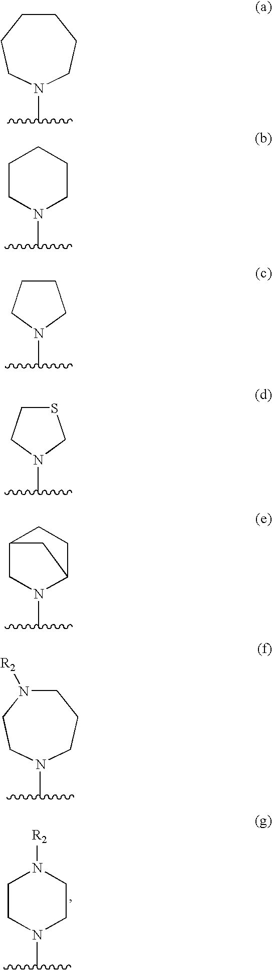

wherein Cy₂ can be optionally substituted with one or more R₂.

In another embodiment, the invention relates to the compounds of formula I wherein Cy₂ is selected from (a)-(f):

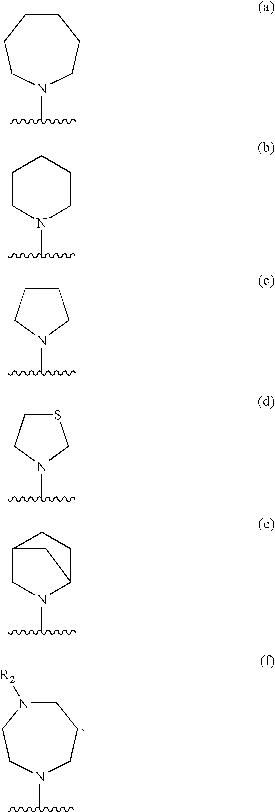

wherein Cy₂ can be optionally substituted with one or more R₂.

In another embodiment, the invention relates to the compounds of formula I wherein Cy₂ is selected from (b), (c), (d), (e), (h) and (i):

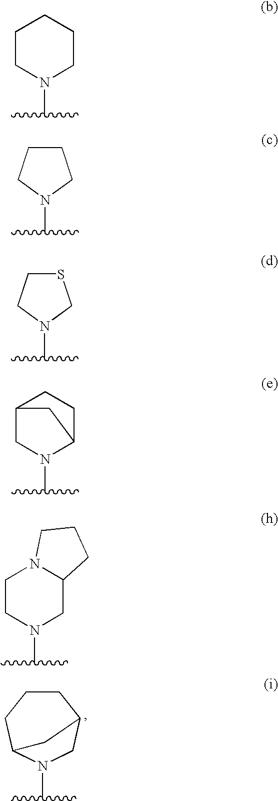

wherein one or more C or S atoms of Cy₂ can be optionally oxidized forming CO, SO or SO₂ groups, and wherein Cy₂ can be optionally substituted with one or more R₂.

In another embodiment, the invention relates to the compounds of formula I wherein Cy₂ represents (b):

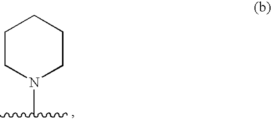

wherein one or more C atoms of Cy₂ can be optionally oxidized forming CO groups, and wherein Cy₂ can be optionally substituted with one or more R₂.

In another embodiment, the invention relates to the compounds of formula I wherein Cy₂ represents (b):

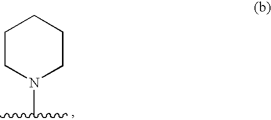

which can be optionally substituted with one or more R₂.

In another embodiment, the invention relates to the compounds of formula I wherein Cy₂ represents (c):

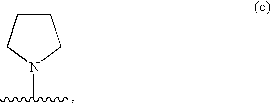

wherein one or more C atoms of Cy₂ can be optionally oxidized forming CO groups, and wherein Cy₂ can be optionally substituted with one or more R₂.

In another embodiment, the invention relates to the compounds of formula I wherein Cy₂ represents (c):

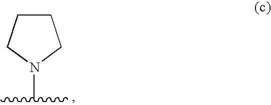

which can be optionally substituted with one or more R₂.

In another embodiment, the invention relates to the compounds of formula I wherein Cy₂ represents (d)

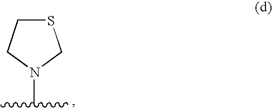

wherein one or more C or S atoms of Cy₂ can be optionally oxidized forming CO, SO or SO₂ groups, and wherein Cy₂ can be optionally substituted with one or more R₂.

In another embodiment, the invention relates to the compounds of formula I wherein Cy₂ represents (d)

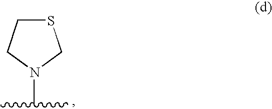

which can be optionally substituted with one or more R₂.

In another embodiment, the invention relates to the compounds of formula I wherein Cy₂ represents (e):

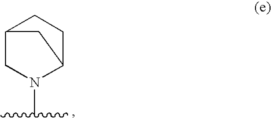

wherein one or more C atoms of Cy₂ can be optionally oxidized forming CO groups, and wherein Cy₂ can be optionally substituted with one or more R₂.

In another embodiment, the invention relates to the compounds of formula I wherein Cy₂ represents (e):

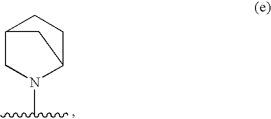

which can be optionally substituted with one or more R₂.

In another embodiment, the invention relates to the compounds of formula I wherein Cy₂ represents (h):

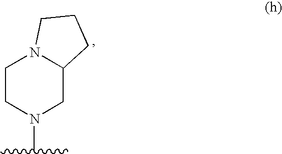

wherein one or more C atoms of Cy₂ can be optionally oxidized forming CO groups, and wherein Cy₂ can be optionally substituted with one or more R₂.

In another embodiment, the invention relates to the compounds of formula I wherein Cy₂ represents (h):

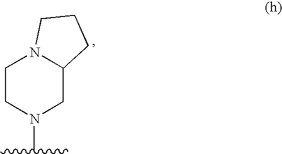

which can be optionally substituted with one or more R₂.

In another embodiment, the invention relates to the compounds of formula I wherein Cy₂ represents (i):

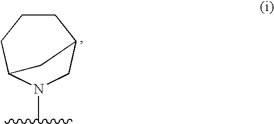

wherein one or more C atoms of Cy₂ can be optionally oxidized forming CO groups, and wherein Cy₂ can be optionally substituted with one or more R₂.

In another embodiment, the invention relates to the compounds of formula I wherein Cy₂ represents (i):

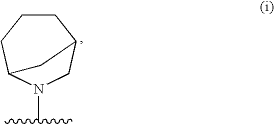

which can be optionally substituted with one or more R₂.

In another embodiment, the invention relates to the compounds of formula I wherein Cy₂ is optionally substituted with one, two, three or four R₂.

In another embodiment, the invention relates to the compounds of formula I wherein each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₃ in R₂ represents Cy_{3c}, and Cy_{3c} represents a saturated 3- to 7-membered monocyclic or 6- to 11-membered bicyclic ring which can be carbocyclic or heterocyclic, in which case it can contain from 1 to 4 heteroatoms selected from N, S and O, wherein Cy_{3c} can be bonded to the rest of the molecule through any available C or N atom, wherein one or more C or S atoms of the ring can be optionally oxidized forming CO, SO or SO₂ groups, and wherein Cy_{3c} can be optionally substituted with one or more R₇.

In another embodiment, the invention relates to the compounds of formula I wherein each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy_{3c} can be optionally substituted with one or more R₇.

In another embodiment, the invention relates to the compounds of formula I wherein each R₂ represents C₁₋₄alkyl, C₁₋₄alkoxyC₁₋₄alkyl, hydroxyC₁₋₄alkyl, haloC₁₋₄alkyl, Cy₄-C₁₋₄alkyl, R₉CO—C₁₋₄alkyl, NR₉R₉—C₁₋₄alkyl, R₉CONR₅—C₁₋₄alkyl, R₁₀SO₂NR₅—C₁₋₄alkyl, NR₉R₉CO—C₁₋₄alkyl, NR₉R₉CONR₅—C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein Cy₃ can be optionally substituted with one or more R₇ and wherein Cy₄ can be optionally substituted with one or more R₈.

In another embodiment, the invention relates to the compounds of formula I wherein each R₂ represents C₁₋₄alkyl, C₁₋₄alkoxyC₁₋₄alkyl, hydroxyC₁₋₄alkyl, haloC₁₋₄alkyl, Cy₄-C₁₋₄alkyl, R₉CO—C₁₋₄alkyl, NR₉R₉—C₁₋₄alkyl, R₉CONR₅—C₁₋₄alkyl, R₁₀SO₂NR₅—C₁₋₄alkyl, NR₉R₉CO—C₁₋₄alkyl, NR₉R₉CONR₅—C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein Cy_{3c} can be optionally substituted with one or more R₇ and wherein Cy₄ can be optionally substituted with one or more R₈.

In another embodiment, the invention relates to the compounds of formula I wherein each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein each R₂ represents C₁₋₄alkyl, C₁₋₄alkoxyC₁₋₄alkyl, hydroxyC₁₋₄alkyl, haloC₁₋₄alkyl, Cy₄-C₁₋₄alkyl, R₉CO—C₁₋₄alkyl, NR₉R₉—C₁₋₄alkyl, R₉CONR₅—C₁₋₄alkyl, R₁₀SO₂NR₅—C₁₋₄alkyl, NR₉R₉CO—C₁₋₄alkyl, NR₉R₉CONR₅—C₁₋₄alkyl, —COR₉, —OR₃, —NR₃R₃, —NR₅COR₃, —NR₅CONR₃R₃ or —NR₅SO₂R₄, wherein Cy₄ can be optionally substituted with one or more R₈.

In another embodiment, the invention relates to the compounds of formula I wherein each R₂ represents C₁₋₄alkyl, C₁₋₄alkoxyC₁₋₄alkyl, hydroxyC₁₋₄alkyl, haloC₁₋₄alkyl, halogen, —CN, —COR₃, —CO₂R₃, —CONR₃R₃, —OR₃, —NR₃R₃, —NR₅COR₃ or Cy₃, wherein Cy₃ can be optionally substituted with one or more R₇.

In another embodiment, the invention relates to the compounds of formula I wherein each R₂ represents C₁₋₄alkyl, C₁₋₄alkoxyC₁₋₄alkyl, hydroxyC₁₋₄alkyl, haloC₁₋₄alkyl, halogen, —CN, —COR₃, —CO₂R₃, —CONR₃R₃, —OR₃, —NR₃R₃, —NR₅COR₃ or Cy₃, wherein Cy₃, can be optionally substituted with one or more R₇.

In another embodiment, the invention relates to the compounds of formula I wherein each R₂ represents C₁₋₄alkyl, hydroxyC₁₋₄alkyl, haloC₁₋₄alkyl, halogen, —COR₃, —CONR₃R₃, —OR₃ or —NR₃R₃.

In another embodiment, the invention relates to the compounds of formula I wherein R₃ in R₂ represents hydrogen or R₄ and R₄ in R₂ represents C₁₋₄alkyl optionally substituted with one or more R₆.

In another embodiment, the invention relates to the compounds of formula I wherein R₃ in R₂ represents hydrogen or R₄ and R₄ in R₂ represents C₁₋₄alkyl, hydroxyC₁₋₄alkyl, C₁₋₄alkoxyC₁₋₄alkyl or haloC₁₋₄alkyl.

In another embodiment, the invention relates to the compounds of formula I wherein:

Cy₂ represents a 5- to 7-membered monocyclic or 6- to 11-membered bicyclic heterocycle, wherein the ring which contains the N atom which is bonded to the pyrrolopyrimidine moiety is saturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein:

Cy₂ represents a 5- to 7-membered monocyclic or 6- to 11-membered bicyclic heterocycle, wherein the ring which contains the N atom which is bonded to the pyrrolopyrimidine moiety is saturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂; and

each R₂ represents C₁₋₄alkyl, C₁₋₄alkoxyC₁₋₄alkyl, hydroxyC₁₋₄alkyl, haloC₁₋₄alkyl, halogen, —CN, —COR₃, —CO₂R₃, —CONR₃R₃, —OR₃, —NR₃R₃, —NR₅COR₃ or Cy₃, wherein Cy₃ can be optionally substituted with one or more R₇.

In another embodiment, the invention relates to the compounds of formula I wherein:

Cy₂ represents a 5- to 7-membered monocyclic or 6- to 11-membered bicyclic heterocycle, wherein the ring which contains the N atom which is bonded to the pyrrolopyrimidine moiety is saturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein:

Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein:

Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂; and

each R₂ represents C₁₋₄alkyl, C₁₋₄alkoxyC₁₋₄alkyl, hydroxyC₁₋₄alkyl, haloC₁₋₄alkyl, halogen, —CN, —COR₃, —CO₂R₃, —CONR₃R₃, —OR₃, —NR₃R₃, —NR₅COR₃ or Cy₃, wherein Cy₃ can be optionally substituted with one or more R₇.

In another embodiment, the invention relates to the compounds of formula I wherein:

Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein:

Cy₂ is selected from (a)-(i):

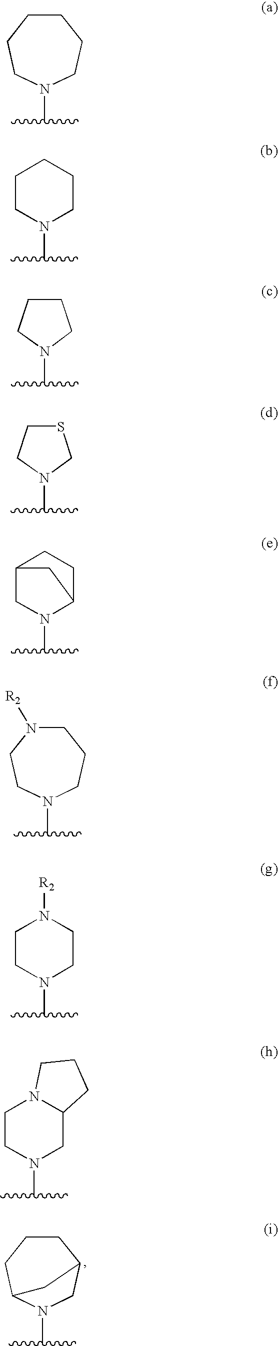

wherein one or more C or S atoms of Cy₂ can be optionally oxidized forming CO, SO or SO₂ groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein:

Cy₂ is selected from (a)-(i):

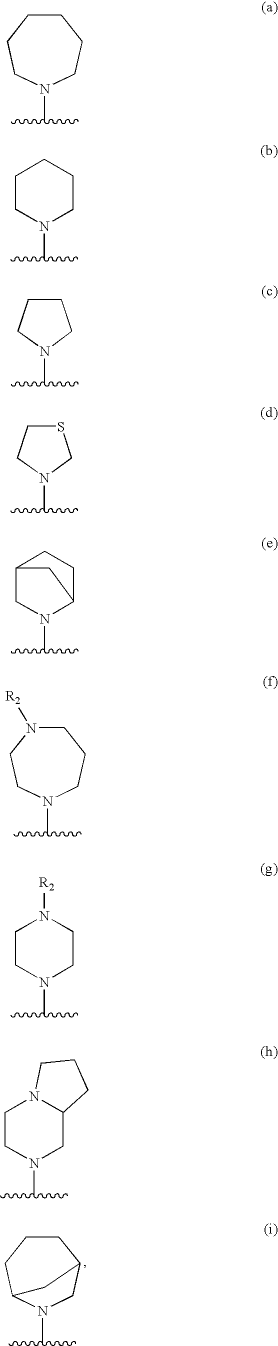

wherein one or more C or S atoms of Cy₂ can be optionally oxidized forming CO, SO or SO₂ groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein:

Cy₂ is selected from (a)-(g):

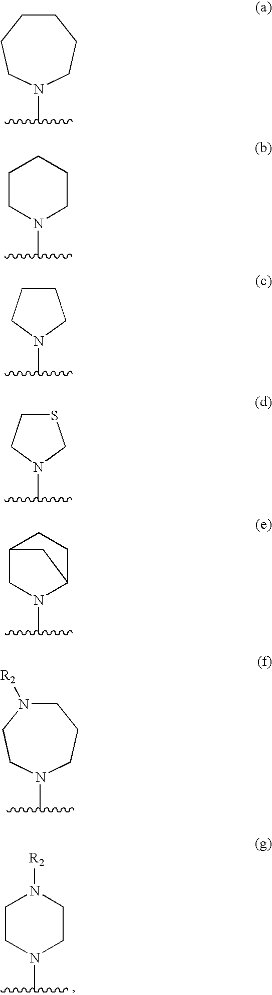

wherein Cy₂ can be optionally substituted with one or more R₂; and

each R₂ represents C₁₋₄alkyl, C₁₋₄alkoxyC₁₋₄alkyl, hydroxyC₁₋₄alkyl, haloC₁₋₄alkyl, halogen, —CN, —COR₃, —CO₂R₃, —CONR₃R₃, —OR₃, —NR₃R₃, —NR₅COR₃ or Cy₃, wherein Cy₃ can be optionally substituted with one or more R₇.

In another embodiment, the invention relates to the compounds of formula I wherein Cy₂ is selected from (b), (c), (d), (e), (h) and (i):

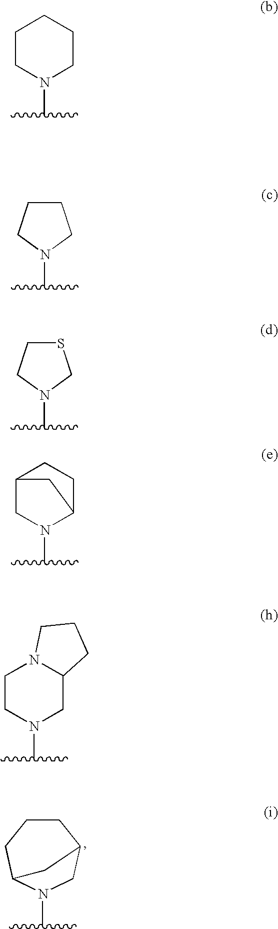

wherein one or more C or S atoms of Cy₂ can be optionally oxidized forming CO, SO or SO₂ groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ is selected from (b), (c), (d), (e), (h) and (i):

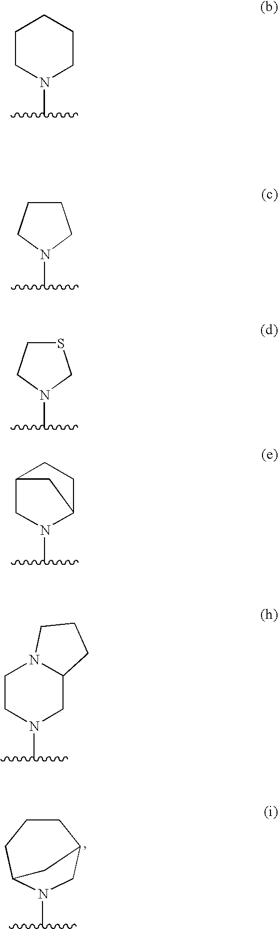

wherein one or more C or S atoms of Cy₂ can be optionally oxidized forming CO, SO or SO₂ groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein Cy₂ represents (b):

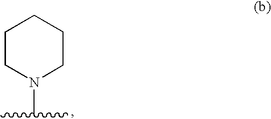

wherein one or more C atoms of Cy₂ can be optionally oxidized forming CO groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ represents (b):

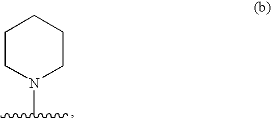

wherein one or more C atoms of Cy₂ can be optionally oxidized forming CO groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein Cy₂ represents (c):

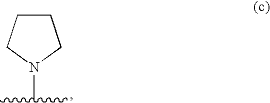

wherein one or more C atoms of Cy₂ can be optionally oxidized forming CO groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ represents (c):

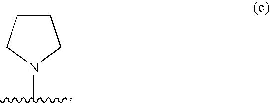

wherein one or more C atoms of Cy₂ can be optionally oxidized forming CO groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein Cy₂ represents (d)

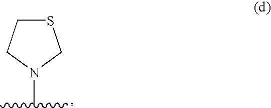

wherein one or more C or S atoms of Cy₂ can be optionally oxidized forming CO, SO or SO₂ groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ represents (d)

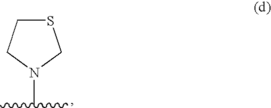

wherein one or more C or S atoms of Cy₂ can be optionally oxidized forming CO, SO or SO₂ groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein Cy₂ represents (e):

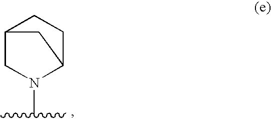

wherein one or more C atoms of Cy₂ can be optionally oxidized forming CO groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ represents (e):

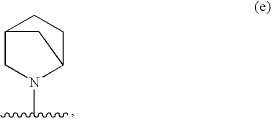

wherein one or more C atoms of Cy₂ can be optionally oxidized forming CO groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein Cy₂ represents (h)

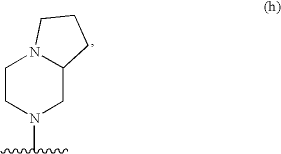

wherein one or more C atoms of Cy₂ can be optionally oxidized forming CO groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ represents (h)

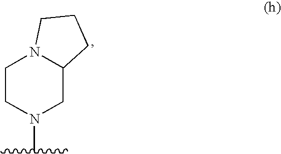

wherein one or more C atoms of Cy₂ can be optionally oxidized forming CO groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein Cy₂ represents (i)

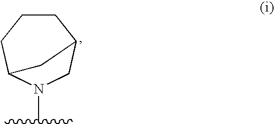

wherein one or more C atoms of Cy₂ can be optionally oxidized forming CO groups, and wherein Cy₂ can be optionally substituted with one or more R₂; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein Cy₂ represents (i)

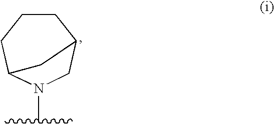

wherein one or more C atoms of Cy₂ can be optionally oxidized forming CO groups, and wherein Cy₂ can be optionally substituted with one or more R₂; and

each R₂ represents C₁₋₄alkyl, —COR₃, —OR₃, —NR₃R₃, —NR₅COR₃, —NR₅CONR₃R₃ or —NR₅SO₂R₄, wherein C₁₋₄alkyl can be optionally substituted with one or more R₆.

In another embodiment, the invention relates to the compounds of formula I wherein:

Cy₁ represents phenyl substituted with one or more R₁; and

Cy₂ represents a 5- to 7-membered monocyclic or 6- to 11-membered bicyclic heterocycle, wherein the ring which contains the N atom which is bonded to the pyrrolopyrimidine is saturated or partially unsaturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ represents a 5- to 7-membered monocyclic or 6- to 11-membered bicyclic heterocycle, wherein the ring which contains the N atom which is bonded to the pyrrolopyrimidine moiety is saturated, wherein Cy₂ contains from 1 to 4 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ represents a 5- to 7-membered saturated monocyclic heterocycle which contains from 1 to 2 heteroatoms selected from N, O and S, wherein one or more C or S atoms can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ is selected from (a)-(g):

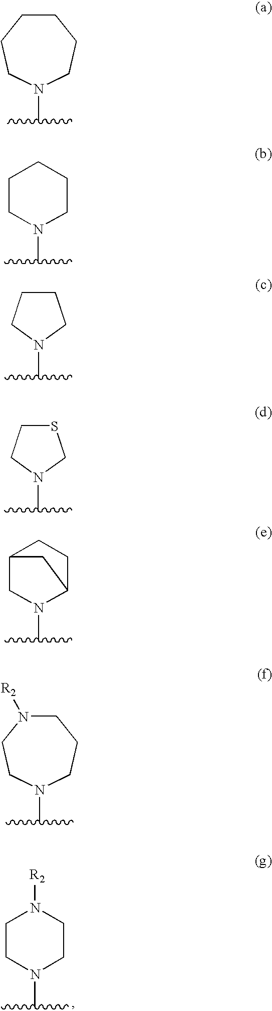

wherein Cy₂ can be optionally substituted with one or more R₂.

In another embodiment, the invention relates to the compounds of formula I wherein:

Cy₁ represents phenyl substituted with one or more R₁; and

Cy₂ is selected from (a)-(i):

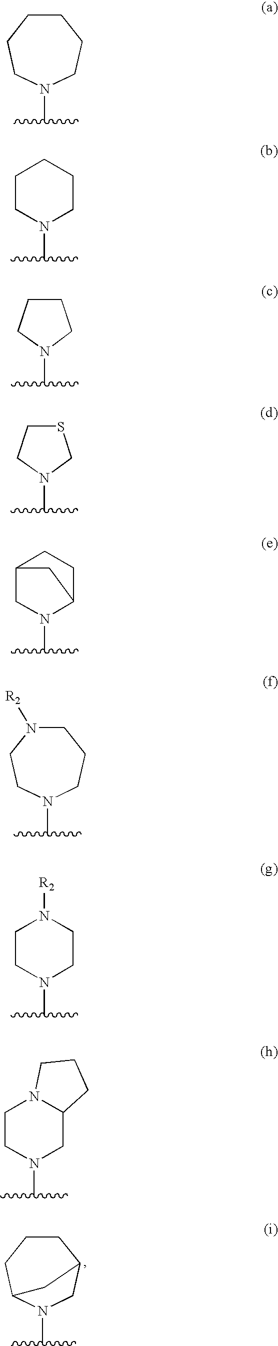

wherein one or more C or S atoms of Cy₂ can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ is selected from (b), (c), (d), (e), (h) and (i):

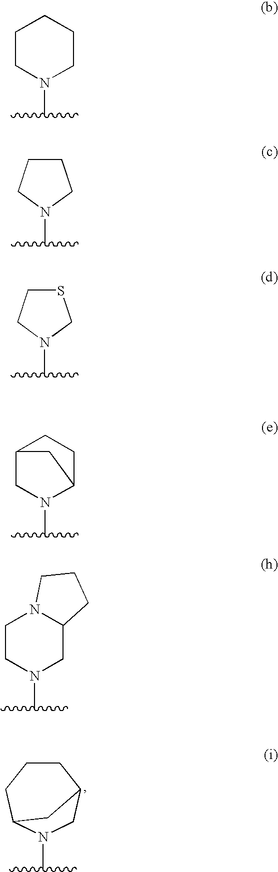

wherein one or more C or S atoms of Cy₂ can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ represents (b):

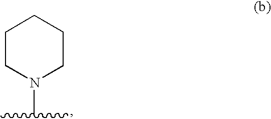

wherein one or more C atoms of Cy₂ can be optionally oxidized forming CO groups, and wherein Cy₂ can be optionally substituted with one or more R₂.

In another embodiment, the invention relates to the compounds of formula I wherein:

Cy₁ represents phenyl substituted with one or more R₁; and

Cy₂ represents (c):

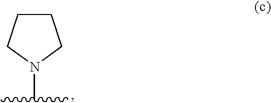

wherein one or more C atoms of Cy₂ can be optionally oxidized forming CO groups, and wherein Cy₂ can be optionally substituted with one or more R₂.

In another embodiment, the invention relates to the compounds of formula I wherein:

Cy₁ represents phenyl substituted with one or more R₁; and

Cy₂ represents (d)

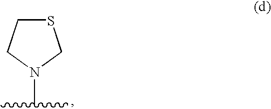

wherein one or more C or S atoms of Cy₂ can be optionally oxidized forming CO, SO or SO₂ groups, and wherein Cy₂ can be optionally substituted with one or more R₂.

In another embodiment, the invention relates to the compounds of formula I wherein:

Cy₁ represents phenyl substituted with one or more R₁; and

Cy₂ represents (e):

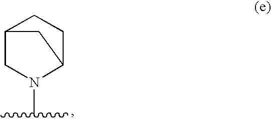

wherein one or more C atoms of Cy₂ can be optionally oxidized forming CO groups, and wherein Cy₂ can be optionally substituted with one or more R₂.

In another embodiment, the invention relates to the compounds of formula I wherein:

Cy₁ represents phenyl substituted with one or more R₁; and

Cy₂ represents (h)

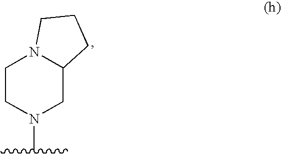

wherein one or more C atoms of Cy₂ can be optionally oxidized forming CO groups, and wherein Cy₂ can be optionally substituted with one or more R₂.

In another embodiment, the invention relates to the compounds of formula I wherein:

Cy₁ represents phenyl substituted with one or more R₁; and

Cy₂ represents (i):

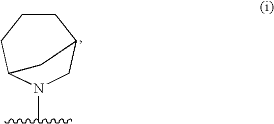

wherein one or more C atoms of Cy₂ can be optionally oxidized forming CO groups, and wherein Cy₂ can be optionally substituted with one or more R₂.

In another embodiment, the invention relates to the compounds of formula I wherein:

Cy₁ represents phenyl substituted with one or more R₁;

Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂;

each R₁ represents C₁₋₄alkyl, halogen, —CONR₃R₃, —OR₃, —SO₂NR₃R₃, —SO₂NR₅COR₄, —NR₅COR₃ or Cy₃, wherein the C₁₋₄alkyl group can be optionally substituted with one or more R₆ and Cy₃ can be optionally substituted with one or more R₇; and

each R₂ represents C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄alkyl can be optionally substituted with one or more R₆ and wherein Cy₃ can be optionally substituted with one or more R₇.

In another embodiment, the invention relates to the compounds of formula I wherein:

Cy₁ represents phenyl substituted with one or more R₁;

Cy₂ represents a saturated 5- to 7-membered monocyclic or 6- to 11-membered bicyclic heterocycle, wherein Cy₂ contains from 1 to 3 heteroatoms selected from N, O and S, wherein one or more C or S atoms can be optionally oxidized forming CO, SO or SO₂ groups, and wherein Cy₂ can be optionally substituted with one or more R₂;

each R₁ represents C₁₋₄alkyl, hydroxyC₁₋₄alkyl, C₁₋₄alkoxyC₁₋₄alkyl, Cy₄-C₁₋₄alkyl, NR₉R₉SO₂—C₁₋₄alkyl, NR₉R₉CO—C₁₋₄alkyl, R₁₀CONR₅SO₂—C₁₋₄alkyl, R₉CONR₅—C₁₋₄alkyl, halogen, —CONR₃R₃, —OR₃, —SO₂NR₃R₃, —SO₂NR₅COR₄, —NR₅COR₃ or Cy₃, wherein Cy₃ can be optionally substituted with one or more R₇ and wherein Cy₄ can be optionally substituted with one or more R₈; and

each R₂ represents C₁₋₄alkyl, C₁₋₄alkoxyC₁₋₄alkyl, hydroxyC₁₋₄alkyl, haloC₁₋₄alkyl, Cy₄-C₁₋₄alkyl, R₉CO—C₁₋₄alkyl, NR₉R₉—C₁₋₄alkyl, R₉CONR₅—C₁₋₄alkyl, R₁₀SO₂NR₅—C₁₋₄alkyl, NR₉R₉CO—C₁₋₄alkyl, NR₉R₉CONR₅—C₁₋₄alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein Cy₃ can be optionally substituted with one or more R₇ and wherein Cy₄ can be optionally substituted with one or more R₈.

Furthermore, the present invention covers all possible combinations of the particular and preferred embodiments described above.

In another embodiment, the invention relates to a compound of formula I, which provides more than 50% inhibition of JAK3 activity at 10 μM, more preferably at 1 μM and still more preferably at 0.1 μM, in a JAK3 assay such as the one described in example 14.

In another embodiment, the invention relates to a compound of formula I selected from the list of compounds described in examples 1 to 13.

The compounds of the present invention contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when they are used for therapeutic purposes. The term pharmaceutically acceptable salt represents those salts which are, according to medical judgment, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in the conventional manner. The salts of the compounds of formula I can be converted into other salts of the compounds of formula I by ion exchange using ionic exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof) and a solvent. Examples of solvents include pharmaceutically acceptable solvents such as water, ethanol and the like. A complex with water is known as a hydrate. Solvates of compounds of the invention (or salts thereof), including hydrates, are included within the scope of the invention.

The compounds of formula I may exist in different physical forms, i.e. amorphous and crystalline forms. Moreover, the compounds of the invention may have the ability to crystallize in more than one form, a characteristic which is known as polymorphism. Polymorphs can be distinguished by various physical properties well known in the art such as X-ray diffraction pattern, melting point or solubility. All physical forms of the compounds of formula I, including all polymorphic forms (“polymorphs”) thereof, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of formula I. Optically pure isomers can also be individually obtained using enantiospecific synthesis. The present invention covers all individual isomers as well as mixtures thereof (for example racemic mixtures or mixtures of diastereomers), whether obtained by synthesis or by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups with conventional protecting groups. Both the nature of these protecting groups and the procedures for their introduction and removal are well known in the art (see for example Greene T. W. and Wuts P. G. M, “Protecting Groups in Organic Synthesis”, John Wiley & Sons, 3^{rd} edition, 1999). As an example, as protecting group of an amino function the tert-butoxycarbonyl (BOC) group can be used. Whenever a protecting group is present, a later deprotection step will be required, which can be performed under standard conditions in organic synthesis, such as those described in the above-mentioned reference.

Unless otherwise stated, in the methods described below the meanings of the different substituents are the meanings described above with regard to a compound of formula I.

In general, compounds of formula I can be obtained in two steps by the method described in Scheme 1:

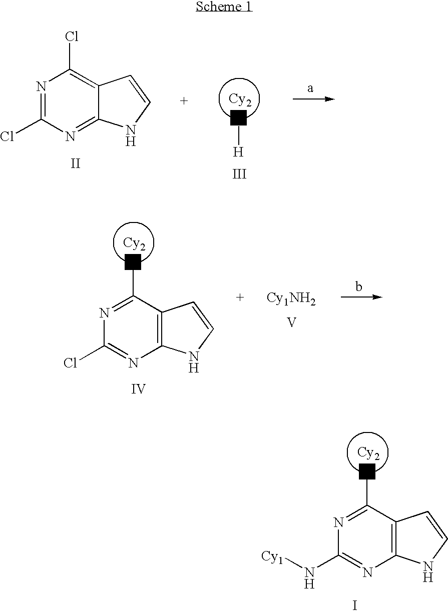

wherein Cy₁ and Cy₂ have the meaning previously described in relation with a compound of formula I.

In a first step (step a), the reaction between a compound of formula II and a compound of formula III may be carried out in the presence of a base such as triethylamine, K₂CO₃, Cs₂CO₃ or diisopropylethylamine, a solvent such as ethanol, tetrahydrofuran/H₂O or any polar solvent, and heating preferably at reflux to obtain a compound of formula IV.

Step b may be carried out by the reaction between a compound of formula IV and an amine of formula V in the presence of 4M dioxane/HCl_{(g)} solution, a solvent such as n-butanol or methoxyethanol, and irradiating with a microwave oven preferably at around 170° C. to obtain a compound of formula I.

Alternatively, step b may be carried out by the reaction between a compound of formula IV and an amine of formula V in the presence of a Pd catalyst such as Pd₂(dba)₃, a phosphine such as 2-dicyclohexylphosphino-2′,4′,6′-triisopropylbiphenyl, and a base such as potassium carbonate, in a solvent such as tert-butanol, and heating preferably at reflux to obtain a compound of formula I.

The compounds of formula II, III and V are commercially available or can be prepared by well-known methods described in the literature, and can be protected with suitable protecting groups.

Furthermore, some compounds of the present invention can also be obtained from other compounds of formula I by appropriate conversion reactions of functional groups in one or several steps, using well-known reactions in organic chemistry under the standard experimental conditions.

Said transformations can be carried out upon Cy₁ or Cy₂ groups and include, for example:

the reduction of a nitro group to give an amino group, for example by treatment with hydrogen, hydrazine or formic acid in the presence of a suitable catalyst such as Pd/C; or by treatment with sodium borohydride in the presence of NiCl₂, or SnCl₂;

the substitution of a primary or secondary amine by treatment with an alkylating agent under standard conditions, or by reductive amination, i.e. by treatment with an aldehyde or a ketone in the presence of a reducing agent such as sodium cyanoborohydride or sodium triacetoxyborohydride;

the conversion of an amine into a sulfonamide by reaction with a sulfonyl halide, such as sulfonyl chloride, optionally in the presence of catalytic amounts of a base such as 4-dimethylaminopyridine, in a suitable solvent such as dioxane, chloroform, dichloromethane or pyridine, optionally in the presence of a base such as triethylamine or pyridine;

the conversion of an amine into an amide, carbamate or urea under standard conditions;

the alkylation of an amide by treatment with an alkylating agent under basic conditions;

the conversion of an alcohol into an ether, ester or carbamate under standard conditions;

the alkylation of a thiol to give a thioeter under standard conditions;

the partial or total oxidation of an alcohol to give ketones, aldehydes or carboxylic acids under standard oxidizing conditions;

the reduction of an aldehyde or a ketone to an alcohol by treatment with a reducing agent such as sodium borohydride;

the reduction of a carboxylic acid or a carboxylic acid derivative to an alcohol by treatment with a reducing agent such as diisobutylaluminum hydride or LiAlH₄;

the oxidation of a thioeter to a sulfoxide or sulfone under standard conditions;

the conversion of an alcohol into a halogen by reaction with SOCl₂, PBr₃, tetrabutylammonium bromide in the presence of P₂O₅, or PI₃;

the conversion of a halogen atom into an amine by reaction with an amine, optionally in the presence of a suitable solvent, and preferably heating;

the conversion of a primary amide into a —CN group or vice versa, under standard conditions.

Likewise, any of the aromatic rings of the compounds of the present invention can undergo electrophilic aromatic substitution reactions or nucleophilic aromatic substitution reactions, widely described in the literature.

Some of these interconversion reactions are explained in greater detail in the examples.

As it will be obvious to those skilled in the art, these interconversion reactions can be carried out upon the compounds of formula I as well as upon any suitable synthesis intermediate thereof.

As mentioned above, the compounds of the present invention act by inhibiting JAK/STAT signaling pathways, particularly by inhibiting JAK3 activity. Therefore, the compounds of the invention are expected to be useful to treat diseases in which JAKs, particularly JAK3, play a role in mammals, including human beings. These diseases include, but are not limited to, transplant rejection; immune, autoimmune and inflammatory diseases; neurodegenerative diseases; and proliferative disorders (see e.g. O'Shea J. J. et al, Nat. Rev. Drug. Discov. 2004, 3(7):555-64; Cetkovic-Cvrlje M. et al, Curr. Pharm. Des. 2004, 10(15):1767-84; Cetkovic-Cvrlje M. et al, Arch. Immunol. Ther. Exp. (Warsz), 2004, 52(2):69-82).

Acute or chronic transplant rejection reactions that can be treated with the compounds of the present invention include any kind of cell, tissue or organ xenotransplants or allografts, such as of heart, lung, liver, kidney, pancreas, uterus, joints, pancreatic islets, bone marrow, limbs, cornea, skin, hepatocytes, pancreatic beta cells, pluripotential cells, neuronal cells and myocardial cells, as well as graft-versus-host reactions (see e.g. Rousvoal G. et al, Transpl. Int. 2006, 19(12):1014-21; Borie D C. et al, Transplantation 2005, 79(7):791-801; Paniagua R. et al, Transplantation 2005, 80(9):1283-92; Higuchi T. et al, J. Heart Lung Transplant. 2005, 24(10):1557-64; Saemann M D. et al, Transpl Int. 2004, 17(9):481-89; Silva Jr H T. et al, Drugs 2006, 66(13):1665-1684).

Immune, autoimmune and inflammatory diseases that can be treated with the compounds of the present invention include among others, rheumatic diseases (e.g. rheumatoid arthritis and psoriatic arthritis), autoimmune hematological disorders (e.g. hemolytic anemia, aplastic anemia, idiopathic thrombocytopenia, and neutropenia), autoimmune gastritis and inflammatory bowel diseases (e.g. ulcerative colitis and Crohn's disease), scleroderma, type I diabetes and complications from diabetes, type B hepatitis, type C hepatitis, primary biliary cirrhosis, myasthenia gravis, multiple sclerosis, systemic lupus erythematosus, psoriasis, atopic dermatitis, contact dermatitis, eczema, skin sunburns, suppression of HIV replication, infertility of autoimmune origin, autoimmune thyroid disease (Grave's disease), interstitial cystitis, and mast cell-mediated allergic reactions such as asthma, angioedema, anaphylaxis, bronchitis, rhinitis and sinusitis (see e.g. Sorbera L A. et al, Drugs of the Future 2007, 32(8):674-680; O′Shea J. J. et al, Nat. Rev. Drug. Discov. 2004, 3(7):555-64; Cetkovic-Cvrlje M. et al, Curr. Pharm. Des. 2004, 10(15):1767-84; Muller-Ladner U. et al, J. Immunol. 2000, 164(7): 3894-3901; Walker J G. et al, Ann. Rheum. Dis. 2006, 65(2):149-56; Milici A J. et al, Arthritis Rheum. 2006, 54 (9, Suppl): abstr 789; Kremer J M. et al, Arthritis Rheum. 2006, 54, 4116, presentation no. L40; Cetkovic-Cvrlje M. et al, Arch Immunol. Ther. Exp. (Warsz), 2004, 52(2):69-82; Malaviya R. et al, J. Pharmacol. Exp. Ther. 2000, 295(3):912-26; Malaviya R. et al, J. Biol. Chem. 1999, 274(38):27028-38; Wilkinson B et al, Ann. Rheum. Dis. 2007, 66(Suppl 2): Abst. THU0099; Matsumoto M. et al, J. Immunol. 1999, 162(2):1056-63).

Neurodegenerative diseases that can be treated with the compounds of the present invention include, among others, amyotrophic lateral sclerosis and Alzheimer's disease (see e.g. Trieu V N. et al, Biochem. Biophys. Res. Commun. 2000, 267(1):22-5).

Proliferative disorders that can be treated with the compounds of the present invention include, among others, leukemias, lymphomas, glioblastoma multiforme, colon carcinoma, as well as thromboembolic and allergic complications associated with these diseases (see e.g. Sudbeck E A. et al, Clin. Cancer Res. 1999, 5(6):1569-82; Narla R K. et al, Clin. Cancer Res. 1998, 4(10):2463-71; Lin Q. et al, Am J. Pathol. 2005, 167(4):969-80; Tibbles H E. et al, J. Biol. Chem. 2001, 276(21):17815-22).

Biological assays that can be used to determine the ability of a compound to inhibit JAKs, particularly JAK3, are well known in the art. For example, a compound to be tested can be incubated in the presence of JAK3 to determine whether inhibition of JAK3 enzymatic activity occurs, as described in the assay of example 14. Other in vitro useful assays that can be used to measure JAK3-inhibitory activity include cellular assays, for example IL-2-induced proliferation of human T lymphocytes. The immunosuppressive activity of the compounds of the invention can be tested using standard in vivo animal models for immune and autoimmune diseases, which are well known in the art. For example, the following assays can be used: delayed-type hypersensitivity (DTH) (see e.g. the method disclosed in Kudlacz E. et al, Am J. Transplant. 2004, 4(1):51-7, the contents of which are incorporated herein by reference), rheumatoid arthritis models such as collagen-induced arthritis (see e.g. the method disclosed in Holmdahl R et al, APMIS, 1989, 97(7):575-84, the contents of which are incorporated herein by reference), multiple sclerosis models such as experimental autoimmune encephalomyelitis (EAE) (see e.g. the method disclosed in González-Rey et al, Am. J. Pathol. 2006, 168(4): 1179-88, the contents of which are incorporated herein by reference) and transplant rejection models (see e.g. the various animal models disclosed in the references listed above in relation to the treatment of transplant rejection, incorporated herein by reference).

For selecting active compounds, testing at 10 μM must result in an activity of more than 50% inhibition of JAK3 activity in the test provided in example 14. More preferably, when tested in this assay compounds should exhibit more than 50% inhibition at 1 μM, and still more preferably, they should exhibit more than 50% inhibition at 0.1 μM.

The present invention also relates to a pharmaceutical composition that comprises a compound of the present invention (or a pharmaceutically acceptable salt or solvate thereof) and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the addition of water can be obtained by mixing the active compound with dispersing or wetting agents; suspending agents and preservatives. Other excipients can also be added, for example sweetening, flavoring and colouring agents.

Liquid forms for oral administration include emulsions, solutions, suspensions, syrups and elixirs containing commonly used inert diluents, such as purified water, ethanol, sorbitol, glycerol, polyethylene glycols (macrogols) and propylene glycol. Said compositions can also contain coadjuvants such as wetting, suspending, sweetening, flavoring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions, which will be dissolved in water or any other sterile injectable medium immediately before use. It is also possible to start from sterile materials and keep them under these conditions throughout all the manufacturing process.

For the rectal administration, the active compound can be preferably formulated as a suppository on an oily base, such as for example vegetable oils or solid semisynthetic glycerides, or on a hydrophilic base such as polyethylene glycols (macrogol).

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as a single or divided doses.

The following examples illustrate the scope of the invention.

### EXAMPLES

The following abbreviations have been used in the examples:

AcN: acetonitrile
n-BuOH: 1-butanol

### DIEA: N,N-diisopropylethylamine

DMAP: 4-(dimethylamino)pyridine

### DMF: N,N-dimethylformamide

EDC: N-[3-(dimethylamino)propyl]-M-ethylcarbodiimide
EtOAc: ethyl acetate
EtOH: ethanol
HBTU: O-(benzotriazol-1-yl)-N,N,N′,N′-tetramethyluronium hexafluorophosphate
HOBT: 1-hydroxybenzotriazole
HPLC: high performance liquid chromatography
LC-MS: liquid chromatography-mass spectroscopy
MeOH: methanol
Pd₂(dba)₃: tris(dibenzylideneacetone)dipalladium(0)
TEA: triethylamine
THF: tetrahydrofuran
t_{R}: retention time
X-Phos: 2-dicyclohexylphosphino-2′,4′,6′-triisopropyl-biphenyl

LC-MS spectra have been performed using the following chromatographic methods:

Method 1: Column X-Terra, MS C18 5 μm (100 mm×2.1 mm), temperature: 30° C., flow: 0.35 mL/min, eluent: A=AcN, B=NH₄HCO₃ 10 mM, gradient: 0 min 10% A; 10 min 90% A; 15 min 90% A; 15.01 min 10% A.
Method 2: Column Waters Acquity UPLC BEH C18 (1.7 μm, 2.1 mm×50 mm), temperature: 40° C., flow: 0.5 mL/min, eluent: ACN (A)/ammonium bicarbonate 10 mM (B), gradient: 0 min 10% A-3.75 min 90% A.
Method 3: Column Tracer Excel 120, ODSB 5 μm (10 mm×0.21 mm), temperature: 30° C., flow: 0.35 mL/min, eluent: A=AcN, B=0.1% HCOOH, gradient: 0 min 10% A-10 min 90% A.
Method 4: Column YMC, 3 μm (50 mm×4.6), temperature: 30° C., flow: 2.6 mL/min, eluent: A=H₂O (0.1% HCOOH) B=AcN (0.1% HCOOH), gradient: 0 min 5% B; 4.8 min 95% B; 6 min 95% B.
Method 5: Column Symmetry C₁₈ 3.5 μm (4.6×75 mm), temperature: 30° C., flow: 1.0 mL/min, eluent: A=H₂O (0.1% HCOOH) B=AcN (0.07% HCOOH), gradient: 0 min 5% B; 7 min 100% B.

### REFERENCE EXAMPLE 1

### [4-(3-Hydroxypiperidin-1-yl)phenyl]amine

### a) 4-(3-Hydroxypiperidin-1-yl)nitrobenzene

To a 4-fluoronitrobenzene solution (1 g, 7.09 mmol) in AcN (16 mL), 3-hydroxypiperidine hydrochloride (1.04 g, 7.57 mmol) and DIEA (1.32 mL, 7.57 mmol) were added. The mixture was stirred and refluxed for 18 h. The resulting mixture was cooled until room temperature and concentrated to dryness. The crude product obtained was chromatographed over silica gel using hexane/EtOAc mixtures of increasing polarity as eluent, to afford 1.15 g of the desired compound (51% yield).

### b) Title Compound

To a NiCl₂.6H₂O (222 mg, 0.93 mmol) suspension in MeOH (50 mL) NaBH₄ (74 mg, 1.95 mmol) was added at room temperature and a solution of the compound obtained in the previous section (0.51 g, 2.33 mmol) in THF (30 mL). The resulting mixture was stirred for 1 h at room temperature and concentrated to dryness. The residue obtained was divided between a 1N NaOH solution and EtOAc. Phases were separated and the aqueous phase was extracted with EtOAc. The combined organic phases were dried over Na₂SO₄ and concentrated to dryness. The crude product obtained was chromatographed over silica gel using hexane/EtOAc mixtures of increasing polarity as eluent, to afford 450 mg of the desired compound (99% yield).

LC-MS (method 1): t_{R}=3.06 min; m/z=193 (MH⁺).

Following a similar procedure to that described in reference example 1, but using in each case the corresponding starting materials, these compounds are obtained:

| Reference | | | HPLC | t_{R} | |
|---|---|---|---|---|---|
| Example | Compound name | Starting Material | method | (min) | m/z |
| 1a | [4-(3-tert- | 3-tert- | 1 | 5.41 | 308 |
| | butoxycarbonylaminopyrrolidin- | butoxycarbonylaminopyrrolidine | | | |
| | 1-yl)phenyl]amine | | | | |
| 1b | [4-(3-hydroxypyirrolidin-1-yl)phenyl]amine | 3-hydroxypyirrolidine | 1 | 2.53 | 179 |

### REFERENCE EXAMPLE 2

### 4-(4-aminophenyl)-1-[2-(trimethylsylanyl)ethoxymethyl]-pyrazol

### a) 4-(4-nitrophenyl)-1-[2-(trimethylsylanyl)ethoxymethyl]-pyrazol

To a 4-(4-nitrophenyl)-1H-pyrazol (0.2 g, 1.05 mmol) and DIEA (0.55 mL, 3.15 mmol) solution in CHCl₃ (3 mL) (2-trimethylsylanyl)ethoxymethyl chloride is added. The resulting mixture was stirred for 18 h at room temperature. H₂O was added and extracted thrice with CHCl₃. The organic phase was dried over Na₂SO₄ and concentrated to dryness. The crude product obtained was chromatographed over silica gel using hexane/EtOAc mixtures of increasing polarity as eluent, to afford 320 mg of the desired compound (95% yield).

### b) Title Compound

Following a similar procedure to that described in reference example 1 section b, but starting with the compound obtained in previous section, the desired compound was obtained (83% yield).

LC-MS (method 1): t_{R}=8.31 min; m/z=290 (MH⁺).

Following a similar procedure to that described in reference example 2, but using in each case the corresponding starting materials, these compounds are obtained:

| Reference | | | HPLC | t_{R} | |
|---|---|---|---|---|---|
| Example | Compound name | Starting material | method | (min) | m/z |
| 2a | 3-(4-aminophenyl)-1-[2- | 3-(4-nitrophenyl)-1H- | 1 | 8.54 | 290.17 |
| | (trimethylsylanyl)ethoxymethyl]- | pyrazol | | | |
| | pyrazol | | | | |

### REFERENCE EXAMPLE 3

### N-(3-Aminophenyl)-N-methylacetamide

### a) N-(3-Nitrophenyl)-N-methylacetamide

To a solution of 3-nitro-N-methylaniline (650 mg, 4.27 mmol) in CH₂Cl₂ (10 mL) under Ar-atmosphere, acetyl chloride (0.33 mL, 4.7 mmol), a catalytic amount of DMAP and DIEA (1.49 mL, 8.5 mmol) were added. The resulting mixture was stirred at room temperature overnight. The resulting residue was diluted with H₂O, the phases were separated and the aqueous phase extracted with CH₂Cl₂. The combined organic phases were dried over Na₂SO₄ and concentrated to dryness. The crude product thus obtained was directly used in the next step.

LC-MS (method 5): t_{R}=1.43 min; m/z=195 (MH⁺).

### b) Title Compound

To a solution of the compound obtained in the previous section (0.96 g, 4.97 mmol) in MeOH (13 mL) under Ar-atmosphere, 10% Pd/C (128 mg) was added at room temperature. The resulting mixture was stirred under H₂ overnight, filtered and the filtrate was concentrated to dryness. The crude product thus obtained was chromatographed over silica gel using Hexane/EtOAc mixtures of increasing polarity as eluent, to afford 0.45 g of the desired compound (56% yield).

LC-MS (method 2): t_{R}=1.02 min; m/z=165 (MH⁺).

Following a similar procedure to that described in reference example 3, but using the corresponding starting material, the following compound was obtained:

| Reference | | Starting | HPLC | t_{R} | |
|---|---|---|---|---|---|
| example | Compound name | material | method | (min) | m/z |
| 3a | N-(3-aminophenyl)-N- | cyclopropane | 5 | 1.38 | 191 |
| | methylcyclopropane- | carbonyl | | | |
| | carboxamide | chloride | | | |

### REFERENCE EXAMPLE 4

### 4-(Imidazol-1-ylmethyl)piperidine

### a) 4-Piperidylmethanol

To a mixture of LiAlH₄ (8.82 g, 0.23 mol) and THF (125 mL), cooled at 0° C., a solution of ethyl isonipecotate (18 mL, 0.117 mol) in THF (325 mL) was added dropwise under Ar-atmosphere, the mixture was stirred at room temperature overnight. A mixture of H₂O (12.03 mL) and THF (25 mL), followed by a mixture of 15% NaOH (10.03 mL) and H₂O (32.4 mL) were slowly added at 0° C. The resulting mixture was washed with THF, filtered and concentrated to dryness. The residue was partitioned between H₂O and CHCl₃, the phases were separated, the aqueous phase was extracted with CHCl₃ and the combined organic phases were dried over Na₂SO₄ and concentrated to afford 8.2 g of the desired product (61% yield).

### b) (1-tert-Butoxycarbonylpiperidin-4-yl)methanol

To a solution of the compound obtained in the previous section (15.3 g, 133 mmol) in DMF (160 mL), at 0° C. and under Ar-atmosphere, di-tert-butyl dicarbonate (29 g, 133 mmol) in DMF (80 mL) was added. The solution was stirred at room temperature overnight, and concentrated to dryness. The residue was dissolved in a mixture of THF (100 mL), MeOH (100 mL) and 1N NaOH (100 mL) and stirred at room temperature for 18 h. The organic phase was evaporated and the aqueous phase was extracted thrice with CHCl₃. The combined organic phases were dried over Na₂SO₄ and concentrated to dryness to afford 23.0 g of the desired product (80% yield).

### c) (1-tert-Butoxycarbonylpiperidin-4-yl)methyl mesylate

To a solution of the product obtained in the previous section (6.8 g, 31 mmol) and DIEA (5.75 mL, 33 mmol) in CH₂Cl₂ (50 mL), at 0° C. and under Ar-atmosphere, methanesulfonyl chloride (2.4 mL, 31 mmol) was added dropwise. The reaction mixture was stirred at room temperature overnight and treated with H₂O, the phases were separated and the aqueous phase was extracted with CH₂Cl₂. The combined organic phases were dried over Na₂SO₄ and concentrated to afford the title compound in quantitative yield.

¹H NMR (300 MHz, CDCl₃) δ (TMS): 4.12 (broad d, J=11.8 Hz, 2H), 4.04 (d, J=6.5 Hz, 2H), 2.98 (s, 3H), 2.69 (broad t, J=12.4 Hz, 2H), 1.89 (m, 1H), 1.72 (broad d, J=12.9 Hz, 2H), 1.43 (s, 9H), 1.25 (m, 2H).

### d) 1-tert-Butoxycarbonyl-4-(imidazol-1-ylmethyl)piperidine

To a solution of the compound obtained in the previous section (400 mg, 1.36 mmol) in THF (5 mL), K₂CO₃ (188 mg, 1.36 mmol) and imidazole (93 mg, 1.36 mmol) were added. The mixture was stirred and refluxed overnight. The crude product obtained was partitioned between EtOAc and 0.05 N aqueous NaOH solution. The phases were separated and the organic phase was dried over Na₂SO₄ and concentrated to dryness. The crude product thus obtained was chromatographed over silica gel using CHCl₃/MeOH/NH₃ mixtures of increasing polarity as eluent, to afford 170 mg of the desired product (47%).

### e) Title Compound

The compound obtained in the previous section (170 mg, 0.64 mmol) and a 4 M dioxane/HCl_{(g)} mixture (5 mL) were mixed in a flask under Ar-atmosphere. The mixture was stirred at room temperature overnight and concentrated to dryness, to afford the title compound in quantitative yield.

¹H NMR (300 MHz, MeOD) δ (TMS): 8.96 (s, 1H), 7.61 (s, 1H), 7.53 (s, 1H), 4.18 (d, J=7.2 Hz, 2H), 3.36-3.32 (m, 2H), 2.95-2.87 (m, 2H), 2.25-2.10 (m, 1H), 1.78-1.74 (m, 2H), 1.49-1.44 (m, 2H).

### REFERENCE EXAMPLE 5

### N-tert-Butyl-N′-(4-piperidylmethyl)urea hydrochloride

### a) 4-Aminomethyl-1-tert-butoxycarbonylpiperidine

To a solution of 4-(aminomethyl)piperidine (100 g, 0.88 mol) in CHCl₃ (550 mL), cooled at 0° C. and under Ar-atmosphere, a solution of di-tert-butyl dicarbonate (98 g, 0.45 mol) in CHCl₃ (350 mL) was added. The resulting mixture was stirred at room temperature for 48 h, washed with H₂O and the aqueous phase extracted with CHCl₃. The combined organic phases were dried over Na₂SO₄ and the solvents were removed to afford 84.5 g of the title compound (88% yield).

¹H NMR (80 MHz, CDCl₃) δ (TMS): 4.11 (broad d, J=13.4 Hz, 2H), 2.69 (m, 4H), 1.45 (s, 9H), 1.8-0.8 (complex signal, 7H).

### b) N-tert-Butyl-N'-[(1-tert-butoxycarbonylpiperidin-4-yl)methyl]urea

To a solution of 4-aminomethyl-1-tert-butoxycarbonylpiperidine (5 g, 23 mmol) obtained in the previous section in DMF (20 mL), tert-butyl isocyanate (2.63 mL, 23 mmol) was added dropwise under Ar-atmosphere. The reaction mixture was stirred at room temperature overnight and concentrated to dryness to afford the desired compound in quantitative yield.

### c) Title Compound

Following a similar procedure to that described in section e of reference example 4, but using the compound obtained in the previous section, the title compound of the example was obtained in quantitative yield.

¹H NMR (300 MHz, CD₃OD) δ (TMS): 4.92 (s, 4H), 3.37 (m, 2H), 2.97 (m, 4H), 1.95 (m, 2H), 1.80 (m, 1H), 1.43 (m, 2H), 1.31 (s, 9H).

### REFERENCE EXAMPLE 6

### 4-Hydroxy-2-methylpiperidine

To a solution of 2-methyl-4-piperidone (250 mg, 2.21 mmol) in MeOH (8 mL), NaBH₄ (175 mg, 4.62 mmol) was added at 0° C. The resulting mixture was stirred at room temperature overnight. The crude product was partitioned between H₂O and EtOAc and the phases were separated. The organic phase was dried over Na₂SO₄ and concentrated to dryness to afford the desired compound in quantitative yield.

LC-MS (method 2): t_{R}=0.31 min; m/z=116 (MH⁺). 1807/78

### REFERENCE EXAMPLE 7

### (6S,8R)-8-Hydroxy-1,4-diazabicyclo[4.3.0]nonane dihydrochloride

### a) (2S,4R)-N-tert-Butoxycarbonylaminoacetyl-4-hydroxyproline methyl ester

To a mixture of N-(tert-butoxycarbonyl)glycine (950 mg, 5.368 mmol), DIEA (2.82 mL, 16.10 mmol) and HBTU (2.07 g, 5.368 mmol) in 30 mL DMF at 0° C., L-4-hydroxyproline methyl ester hydrochloride (995 mg, 5.368 mmol) was added and the suspension thus obtained was stirred at room temperature overnight. The mixture was concentrated to dryness and partitioned between CHCl₃ and 0.2 M NaHCO₃ solution. The combined organic phases were dried over Na₂SO₄ and concentrated to dryness to afford the desired compound (80%).

LC-MS (method 2): t_{R}=1.25 min; m/z=303.3 (MH⁺).

### b) (6S,8R)-2,5-Dioxo-8-hydroxy-1,4-diazabicyclo[4.3.0]nonane

To a solution of the compound obtained in the previous section (1.3 g, 4.30 mmol) in CH₂Cl₂ (2 mL), trifluoroacetic acid (1 mL) was added. The solution was stirred at room temperature for 2 h. A 2N aqueous NaHCO₃ solution (2 mL) was added and the mixture evaporated to dryness. The crude product was diluted with a mixture of CHCl₃/MeOH/NH₃ (10:5:1, 10 mL) and the solution thus obtained was filtered over silica gel. The filtrate was concentrated to dryness to afford the desired compound in quantitative yield.

LC-MS (method 2): t_{R}=0.28 min; m/z=171.2 (MH⁺).

### c) Title Compound

A solution of the compound obtained in the previous section (914 mg, 5.37 mmol) in THF (10 mL) was added to a solution of LiAlH₄ (815 mg, 21.48 mmol) in THF (21 mL) and under Ar-atmosphere. The resulting mixture was refluxed for 1 h and stirred at room temperature overnight. H₂O (0.82 mL), 15% aqueous NaOH solution (0.82 mL) and H₂O (2.45 mL) were added in this order to the resulting solution. The resulting suspension was stirred for 1 h at room temperature and after the addition of THF (9 mL) the resulting solid was filtered and washed with EtOH. The filtrate was neutralized with a mixture of 2N HCl and Dowex 50w×8 (10 g), and stirred at room temperature overnight. The suspension thus obtained was filtered and washed with a mixture of H₂O/MeOH. NH₄OH/25% MeOH (75 mL) was added stirred for 2 h at room temperature. The resulting suspension was filtered and washed with MeOH. The resulting crude product was redissolved in 4M HCl in 1,4-dioxane (5 mL) and concentrated to dryness to afford the desired product (26%).

LC-MS (method 2): t_{R}=0.28 min; m/z=143 (MH⁺).

Following a similar procedure to that described in reference example 7 sections a, b, and c, but using the corresponding starting materials, the next compounds were obtained:

| Reference | | | HPLC | t_{R} | |
|---|---|---|---|---|---|
| Example | Compound name | Reagents for step a) | method | (min) | m/z |
| 7a | (S)-1,4- | L-proline methyl ester | 2 | 0.34 | 127 |
| | diazabicyclo[4.3.0]nonane | hydrochloride | | | |
| | dihydrochloride | N-(tert-butoxycarbonyl)glycine | | | |
| 7b | (6S,3S)-3-methyl- | L-proline methyl ester | 2 | 0.35 | 141 |
| | 1,4- | hydrochloride | | | |
| | diazabicyclo[4.3.0]nonane | N-(tert-butoxycarbonyl)-L-alanine | | | |
| | dihydrochloride | | | | |

### Example 1

### 2-[4-(4-morpholino)phenyl]amino-4-(piperidin-1-yl)-7H-pyrrolo[2,3-d]pyrimidine

### a) 2-Chloro-4-(piperidin-1-yl)-7H-pyrrolo[2,3-d]pyrimidine

To a 2,4-dichloro-7H-pyrrolo[2,3-d]pyrimidine solution (0.16 g, 0.86 mmol) in EtOH (2 mL), piperidine (0.085 mL, 0.86 mmol) and TEA (0.24 mL, 1.7 mmol) were added. The reaction was stirred and refluxed for 18 h. The resulting mixture was cooled until room temperature and evaporated to dryness. The crude product obtained was chromatographed over silica gel using hexane/EtOAc mixtures of increasing polarity as eluent, to afford 0.18 g of the desired compound (88° A) yield).

### b) Title Compound

A mixture of the compound obtained in previous section (90 mg, 0.38 mmol), [4-(4-morpholino)phenyl]amine (123 mg, 0.57 mmol) and a 4M dioxane/HCl_{(g)} solution (0.1 mL) in n-BuOH (2 mL) was irradiated in a microwave oven at 170° C. for 40 min. n-BuOH was evaporated and the residue was purified by preparative HPLC. 26.5 mg (18% yield) of the title compound was obtained

LC-MS (method 1): t_{R}=8.03 min; m/z=379 (MH⁺).

Following a similar procedure to that described in example 1, but using in each case the corresponding starting materials, these compounds are obtained:

| | | Reagent for | Reagent for step | HPLC | t_{R} | |
|---|---|---|---|---|---|---|
| Example | Compound name | step a) | b) | method | (min) | m/z |
| 1a | 4-(4-methylpiperidin- | 4- | [4-(4- | 1 | 8.69 | 393.2 |
| | 1-yl)-2-[4-(4- | methylpiperidine | morpholino)phenyl]amine | | | |
| | morpholino)phenyl]amino- | | | | | |
| | 7H-pyrrolo[2,3- | | | | | |
| | d]pyirimidine | | | | | |
| 1b | 2-(3- | 4- | 3-amino-N-tert- | 1 | 7.62 | 387.1 |
| | aminosulfonylphenyl)amino- | methylpiperidine | butylbenzenesulfonamide | | | |
| | 4-(4- | | | | | |
| | methylpiperidin-1- | | | | | |
| | yl)-7H-pyrrolo[2,3- | | | | | |
| | d]pyrimidine | | | | | |
| 1c | 2-(3-tert- | 4- | 3-amino-N-tert- | 1 | 9.44 | 443.2 |
| | butylaminosulfonylphenyl)amino- | methylpiperidine | butylbenzenesulfonamide | | | |
| | 4-(4- | | | | | |
| | methylpiperidin-1- | | | | | |
| | yl)-7H-pyrrolo[2,3- | | | | | |
| | d]pyrimidine | | | | | |
| 1d | 2-[4-(3- | 4- | Reference | 1 | 8.10 | 393.2 |
| | hydroxypyrrolidin-1- | methylpiperidine | example 1b | | | |
| | yl)phenyl]amino-4- | | | | | |
| | (4-methylpiperidin-1- | | | | | |
| | yl)-7H-pyrrolo[2,3- | | | | | |
| | d]pyirimidine | | | | | |
| 1e | 4-(4-methylpiperidin- | 4- | N-tert- | 1 | 8.14 | 391.2 |
| | 1-yl)-2-[4-(piperidin- | methylpiperidine | butoxycarbonyl-3- | | | |
| | 3-yl)phenyl]amino- | | (4- | | | |
| | 7H-pyrrolo[2,3- | | aminophenyl)piperidine | | | |
| | d]pyrimidine | | | | | |
| 1f | 2-(3- | piperidine | 3- | 1 | 9.56 | 340.1 |
| | methyltiophenyl)amino- | | methyltiophenylamine | | | |
| | 4-(piperidin-1-yl)- | | | | | |
| | 7H-pyrrolo[2,3- | | | | | |
| | d]pyrimidine | | | | | |
| 1g | 2-(3- | piperidine | 3- | 1 | 9.41 | 338.1 |
| | ethoxyphenyl)amino- | | ethoxyphenylamine | | | |
| | 4-(piperidin-1-yl)- | | | | | |
| | 7H-pyrrolo[2,3- | | | | | |
| | d]pyrimidine | | | | | |
| 1h | 2-[4-(3- | 4- | Reference | 1 | 7.50 | 392.2 |
| | aminopyrrolidin-1- | methylpiperidine | example 1a | | | |
| | yl)phenyl]amino-4- | | | | | |
| | (4-methylpiperidin-1- | | | | | |
| | yl)-7H-pyrrolo[2,3- | | | | | |
| | d]pyrimidine | | | | | |
| 1i | 2-(3- | piperidine | 3-amino-N-tert- | 1 | 6.99 | 373.1 |
| | aminosufonilphenyl)amino- | | butylbenzenesulfonamide | | | |
| | 4-(piperidin- | | | | | |
| | 1-yl)-7H-pyrrolo[2,3- | | | | | |
| | d]pyrimidine | | | | | |
| 1j | 2-(3-tert- | piperidine | 3-amino-N-tert- | 1 | 8.87 | 429.1 |
| | butylaminosufonilphenyl)amino- | | butylbenzenesulfonamide | | | |
| | 4- | | | | | |
| | (piperidin-1-yl)-7H- | | | | | |
| | pyrrolo[2,3- | | | | | |
| | d]pyrimidine | | | | | |
| 1k | 2-[4-(3- | piperidine | Reference | 1 | 6.99 | 378.2 |
| | aminopyrrolidin-1- | | example 1a | | | |
| | yl)phenyl]amino-4- | | | | | |
| | (piperidin-1-yl)-7H- | | | | | |
| | pyrrolo[2,3- | | | | | |
| | d]pyrimidine | | | | | |
| 1l | 2-[4-(3-tert- | piperidine | Reference | 1 | 9.69 | 478.2 |
| | butoxycarbonyl | | example 1a | | | |
| | aminopyrrolidin-1- | | | | | |
| | yl)phenyl]amino-4- | | | | | |
| | (piperidin-1-yl)-7H- | | | | | |
| | pyrrolo[2,3- | | | | | |
| | d]pyrimidine | | | | | |
| 1m | 4-(piperidin-1-yl)-2- | piperidine | N-tert- | 1 | 7.45 | 377.2 |
| | [4-(3- | | butoxycarbonyl-3- | | | |
| | piperidinil)phenyl]amino- | | (4- | | | |
| | 7H-pyrrolo[2,3- | | aminophenyl)piperidine | | | |
| | d]pyrimidine | | | | | |
| 1n | 2-[3- | 4- | 3- | 3 | 6.99 | 350.2 |
| | acetylphenyl]amino- | methylpiperidine | acetylphenylamine | | | |
| | 4-(4-methylpiperidin- | | | | | |
| | 1-yl)-7H-pyrrolo[2,3- | | | | | |
| | d]pyrimidine | | | | | |
| 1o | 4-(4-methylpiperidin- | 4- | 3-(piperidin-1- | 1 | 10.75 | 391.2 |
| | 1-yl)-2-[(3-piperidin- | methylpiperidine | yl)phenylamine | | | |
| | 1-yl)phenyl]amino- | | | | | |
| | 7H-pyrrolo[2,3- | | | | | |
| | d]pyrimidine | | | | | |
| 1p | 4-(4-methylpiperidin- | 4- | [3-(4- | 1 | 8.94 | 393.2 |
| | 1-yl)-2-[3-(4- | methylpiperidine | morpholino)phenyl]amine | | | |
| | morpholino)phenyl]amino- | | | | | |
| | 7H-pyrrolo[2,3- | | | | | |
| | d]pyrimidine | | | | | |
| 1q | 2-(3- | 4- | 3- | 1 | 9.37 | 333.1 |
| | cyanophenyl)amino- | methylpiperidine | aminobenzonitrile | | | |
| | 4-(4-methylpiperidin- | | | | | |
| | 1-yl)-7H-pyrrolo[2,3- | | | | | |
| | d]pyrimidine | | | | | |
| 1r | 4-(4-methylpiperidin- | 4- | 3,4- | 1 | 8.60 | 368.1 |
| | 1-yl)-2-(3,4- | methylpiperidine | dimethoxyphenylamine | | | |
| | dimethoxyphenyl)amino- | | | | | |
| | 7H-pyrrolo[2,3- | | | | | |
| | d]pyrimidine | | | | | |
| 1s | 2-[(3,4- | 4- | 3,4- | 1 | 9.22 | 352.1 |
| | methylendioxy)phenyl]amino- | methylpiperidine | (methylendioxy)phenylamine | | | |
| | 4-(4- | | | | | |
| | methylpiperidin-1-yl)- | | | | | |
| | 7H-pyrrolo[2,3- | | | | | |
| | d]pyrimidine | | | | | |
| 1t | 2-[(3- | 4- | (3- | 1 | 7.70 | 365.1 |
| | acetylamino)phenyl]amino- | methylpiperidine | acetylamino)phenylamine | | | |
| | 4-(4- | | | | | |
| | methylpiperidin-1-yl)- | | | | | |
| | 7H-pyrrolo[2,3- | | | | | |
| | d]pyrimidine | | | | | |
| 1u | 2-(4- | 4- | 4-amino-N-tert- | 1 | 7.56 | 387.0 |
| | aminosufonilphenyl)amino- | methylpiperidine | butylbenzenesulfonamide | | | |
| | 4-(4- | | | | | |
| | methylpiperidin-1- | | | | | |
| | yl)-7H-pyrrolo[2,3- | | | | | |
| | d]pyrimidine | | | | | |
| 1v | 2-[(4- | 4- | (4- | 1 | 7.05 | 351.1 |
| | carbamoil)phenyl]amino- | methylpiperidine | carbamoil)phenyl | | | |
| | 4-(4- | | amine | | | |
| | methylpiperidin-1- | | | | | |
| | yl)-7H-pyrrolo[2,3- | | | | | |
| | d]pyrimidine | | | | | |
| 1w | 4-(4- | 1- | 3-amino-N-tert- | 4 | 1.43 | 416.2 |
| | acetylpiperazine-1- | acetylpiperazine | butylbenzenesulfonamide | | | |
| | yl)-2-(3- | | | | | |
| | aminosulfonylphenyl)amino- | | | | | |
| | 7H- | | | | | |
| | pyrrolo[2,3- | | | | | |
| | d]pyrimidine | | | | | |
| 1x | 2-(3- | 2- | 3-amino-N-tert- | 1 | 1.8 | 373.2 |
| | aminosulfonylphenyl)amino- | methylpyrrolidine | butylbenzenesulfonamide | | | |
| | 4-(2-methyl- | | | | | |
| | 1-pyrrolidinil)-7H- | | | | | |
| | pyrrolo[2,3- | | | | | |
| | d]pyrimidine | | | | | |
| 1y | 2-(3- | 1- | 3-amino-N-tert- | 1 | 7.72 | 449.9 |
| | aminosulfonylphenyl)amino- | phenylpiperazine | butylbenzenesulfonamide | | | |
| | 4-(4- | | | | | |
| | phenylpiperazin-1- | | | | | |
| | yl)-7H-pyrrolo[2,3- | | | | | |
| | d]pyrimidine | | | | | |
| 1z | 2-(3- | 4-phenyl- | 3-amino-N-tert- | 1 | 8.31 | 446.9 |
| | aminosulfonylphenyl)amino- | 1,2,3,6- | butylbenzenesulfonamide | | | |
| | 4-(4-phenyl- | tetrahidropyridine | | | | |
| | 1,2,3,6- | | | | | |
| | tetrahidropyridin-1- | | | | | |
| | yl)-7H-pyrrolo[2,3- | | | | | |
| | d]pyrimidine | | | | | |
| 1aa | 2-[4-(2- | homopiperidine | 2-(4- | 1 | 7.73 | 352.0 |
| | hydroxyethyl)phenyl]amino- | | aminophenyl)ethanol | | | |
| | 4- | | | | | |
| | (homopiperidin-1- | | | | | |
| | yl)-7H-pyrrolo[2,3- | | | | | |
| | d]pyrimidine | | | | | |
| 1ab | 2-[4-(3- | homopiperidine | N-tert- | 1 | 7.07 | 391.1 |
| | piperidinyl)phenyl]amino- | | butoxycarbonyl-3- | | | |
| | 4- | | (4- | | | |
| | (homopiperidin-1- | | aminophenyl)piperidine | | | |
| | yl)-7H-pyrrolo[2,3- | | | | | |
| | d]pyrimidine | | | | | |
| 1ac | 2-(3- | homopiperidine | 3-amino-N-tert- | 1 | 7.43 | 387.0 |
| | aminosulfonylphenyl)amino- | | butylbenzenesulfonamide | | | |
| | 4- | | | | | |
| | (homopiperidin-1- | | | | | |
| | yl)-7H-pyrrolo[2,3- | | | | | |
| | d]pyrimidine | | | | | |
| 1ad | 2-(3- | 3- | 3-amino-N-tert- | 4 | 1.42 | 430.2 |
| | aminosulfonylphenyl)amino- | acetamidopiperidine | butylbenzenesulfonamide | | | |
| | 4-(3- | | | | | |
| | acetamidopiperidin- | | | | | |
| | 1-yl)-7H-pyrrolo[2,3- | | | | | |
| | d]pyrimidine | | | | | |
| 1ae | 2-(3- | homopiperazine | 3-amino-N-tert- | 1 | 4.19 | 388.0 |
| | aminosulfonylphenyl)amino- | | butylbenzenesulfonamide | | | |
| | 4- | | | | | |
| | ([1,4]diazepan-1-yl)- | | | | | |
| | 7H-pyrrolo[2,3- | | | | | |
| | d]pyrimidine | | | | | |
| 1af | 2-(3- | 4- | 3-amino- | 4 | 1.32 | 389.2 |
| | aminosulfonylphenyl)amino- | hydroxypiperidine | benzenesulfonamide | | | |
| | 4-(4- | | | | | |
| | hydroxypiperidin-1- | | | | | |
| | yl)-7H-pyrrolo[2,3- | | | | | |
| | d]pyrimidine | | | | | |
| 1ag | 2-(3- | 3- | 3-amino- | 4 | 1.43 | 389.2 |
| | aminosulfonylphenyl)amino- | hydroxypiperidine | benzenesulfonamide | | | |
| | 4-(3- | | | | | |
| | hydroxypiperidin-1- | | | | | |
| | yl)-7H-pyrrolo[2,3- | | | | | |
| | d]pyrimidine | | | | | |
| 1ah | 2-(3- | 4- | 3-amino-N-tert- | 2 | 1.41 | 403 |
| | aminosulfonylphenyl)amino- | hydroxymethylpiperidine | butylbenzenesulfonamide | | | |
| | 4-(4- | | | | | |
| | hydroxymethylpiperidin- | | | | | |
| | 1-yl)-7H- | | | | | |
| | pyrrolo[2,3- | | | | | |
| | d]pyrimidine | | | | | |
| 1ai | 2-(3- | 4- | 3-amino-N-tert- | 4 | 2.58 | 463.2 |
| | aminosulfonylphenyl)amino- | benzylpiperidine | butylbenzenesulfonamide | | | |
| | 4-(4- | | | | | |
| | benzilpiperidin-1-yl)- | | | | | |
| | 7H-pyrrolo[2,3- | | | | | |
| | d]pyrimidine | | | | | |
| 1aj | 2-(3- | 4- | 3-amino-N-tert- | 4 | 2.43 | 449.2 |
| | aminosulfonylphenyl)amino- | phenylpiperidine | butylbenzenesulfonamide | | | |
| | 4-(4- | | | | | |
| | phenylpiperidin-1- | | | | | |
| | yl)-7H-pyrrolo[2,3- | | | | | |
| | d]pyrimidine | | | | | |
| 1ak | 2-(3- | 3- | 3-amino- | 4 | 1.50 | 403.2 |
| | aminosulfonylphenyl)amino- | hydroxymethylpiperidine | benzenesulfonamide | | | |
| | 4-(3- | | | | | |
| | hydroxymethylpiperidin- | | | | | |
| | 1-yl)-7H- | | | | | |
| | pyrrolo[2,3- | | | | | |
| | d]pyrimidine | | | | | |
| 1al | 2-(3- | 2- | 3-amino- | 4 | 1.80 | 385.2 |
| | aminosulfonylphenyl)amino- | azabiciclo[2,2, | benzenesulfonamide | | | |
| | 4-(2- | 1]heptane | | | | |
| | azabiciclo[2,2,1]heptan- | | | | | |
| | 1-yl)-7H- | | | | | |
| | pyrrolo[2,3- | | | | | |
| | d]pyrimidine | | | | | |
| 1am | 2-(3- | perhydroisoquinoline | 3-amino- | 4 | 2.52 | 427.2 |
| | aminosulfonylphenyl)amino- | | benzenesulfonamide | | | |
| | 4- | | | | | |
| | (perhydroisoquinolin- | | | | | |
| | 2-yl)-7H- | | | | | |
| | pyrrolo[2,3- | | | | | |
| | d]pyrimidine | | | | | |
| 1an | 2-[(4-N,N- | 4- | N,N-diethyl-1,4- | 1 | 10.39 | 379.2 |
| | diethylamine)phenyl]amino- | methylpiperidine | phenylendiamine | | | |
| | 4-(4- | | | | | |
| | methylpiperidin-1- | | | | | |
| | yl)-7H-pyrrolo[2,3- | | | | | |
| | d]pyrimidine | | | | | |
| 1ao | 4-(3-methylpiperidin- | 3- | [4-(4- | 1 | 8.64 | 393 |
| | 1-yl)-2-[4-(4- | methylpiperidine | morpholino)phenyl]amine | | | |
| | morpholino)phenyl]amino- | | | | | |
| | 7H-pyrrolo[2,3- | | | | | |
| | d]pyrimidine | | | | | |
| 1ap | 2-(3- | 3-(N-tert- | 3-amino- | 2 | 1.23 | 374.3 |
| | aminosulfonylphenyl)amino- | butoxycarbonyl- | benzenesulfonamide | | | |
| | 4-(3- | N- | | | | |
| | methylaminoazetidin- | methylamino)azetidine | | | | |
| | 1-yl)-7H- | | | | | |
| | pyrrolo[2,3- | | | | | |
| | d]pyrimidine | | | | | |
| 1aq | 2-[4-(3- | piperidine | Reference | 1 | 7.49 | 393.1 |
| | hydroxypiperidin-1- | | example 1 | | | |
| | yl)phenyl]amino-4- | | | | | |
| | (piperidin-1-yl)-7H- | | | | | |
| | pyrrolo[2,3- | | | | | |
| | d]pyrimidine | | | | | |
| 1ar | 2-[4-(3- | 4- | Reference | 1 | 8.13 | 407.1 |
| | hydroxypiperidin-1- | methylpiperidine | example 1 | | | |
| | yl)phenyl]amino-4- | | | | | |
| | (4-methylpiperidin-1- | | | | | |
| | yl)-7H-pyrrolo[2,3- | | | | | |
| | d]pyrimidine | | | | | |
| 1as | 2-(3- | 4- | 3- | 1 | 10.34 | 399.1 |
| | phenylaminophenyl)amino- | methylpiperidine | (phenylaminophenyl)amine | | | |
| | 4-(4- | | | | | |
| | methylpiperidin-1- | | | | | |
| | yl)-7H-pyrrolo[2,3- | | | | | |
| | d]pyrimidine | | | | | |
| 1at | 2-(4- | 4- | 4-aminophenol | 1 | 7.62 | 324.1 |
| | hydroxyphenyl)amino- | methylpiperidine | | | | |
| | 4-(4- | | | | | |
| | methylpiperidin-1- | | | | | |
| | yl)-7H-pyrrolo[2,3- | | | | | |
| | d]pyrimidine | | | | | |
| 1au | 2-[4-(2- | 4- | 2-(4- | 1 | 7.84 | 352.1 |
| | hydroxyethyl)phenyl]amino- | methylpiperidine | aminophenyl)ethanol | | | |
| | 4-(4- | | | | | |
| | methylpiperidin-1- | | | | | |
| | yl)-7H-pyrrolo[2,3- | | | | | |
| | d]pyrimidine | | | | | |
| 1av | 4-(4-methylpiperidin- | 4- | 4-(piperidin-1- | 1 | 10.51 | 391.1 |
| | 1-yl)-2-[4-(piperidin- | methylpiperidine | yl)aniline | | | |
| | 1-yl)phenyl]amino- | | | | | |
| | 7H-pyrrolo[2,3- | | | | | |
| | d]pyrimidine | | | | | |
| 1aw | 4-(4-methylpiperidin- | 4- | 3-(pyiridyn-4- | 1 | 9.00 | 385.1 |
| | 1-yl)-2-[3-(pyridin-4- | methylpiperidine | yl)aniline | | | |
| | yl)phenyl]amino-7H- | | | | | |
| | pyrrolo[2,3- | | | | | |
| | d]pyrimidine | | | | | |
| 1ax | 2-(3- | 4- | 3-aminophenol | 1 | 8.05 | 324.1 |
| | hydroxyphenyl)amino- | methylpiperidine | | | | |
| | 4-(4- | | | | | |
| | methylpiperidine-1- | | | | | |
| | yl)-7H-pyrrolo[2,3- | | | | | |
| | d]pyrimidine | | | | | |
| 1ay | 2-(benzofuran-5- | 4- | (benzofuran-5- | 1 | 9.49 | 348.1 |
| | yl)amino-4-(4- | methylpiperidine | yl)amine | | | |
| | methylpiperidin-1- | | | | | |
| | yl)-7H-pyrrolo[2,3- | | | | | |
| | d]pyrimidine | | | | | |
| 1az | 2-(3- | (R)-3- | 3-amino- | 4 | 1.27 | 375.1 |
| | aminosulfonylphenyl)amino- | hydroxypyrrolidine | benzenosulfonamide | | | |
| | 4-[(R)-3- | | | | | |
| | hydroxypyrrolidin-1- | | | | | |
| | yl]-7H-pyrrolo[2,3- | | | | | |
| | d]pyrimidine | | | | | |
| 1ba | 4-(4-methylpiperidin- | 4- | 4-(1,1- | 1 | 8.10 | 441.1 |
| | 1-yl)-2-[4-(1,1- | methylpiperidine | dioxotiomorpholin- | | | |
| | dioxotiomorpholin-4- | | 4-yl)aniline | | | |
| | yl)phenyl]amino-7H- | | | | | |
| | pyrrolo[2,3- | | | | | |
| | d]pyrimidine | | | | | |
| 1bb | 2-(3- | pyrrolidine | 3-amino-N-tert- | 4 | 1.63 | 359.2 |
| | aminosulfonylphenyl)amino- | | butylbenzenesulfonamide | | | |
| | 4-(pyrrolidin- | | | | | |
| | 1-yl)-7H-pyrrolo[2,3- | | | | | |
| | d]pyrimidine | | | | | |
| 1bc | 4-(4-methylpiperidin- | 4- | 4- | 1 | 8.24 | 386.1 |
| | 1-yl)-2-[4- | methylpiperidine | (methylsulfonyl)aniline | | | |
| | (methylsulfonyl)phenyl]amino- | | | | | |
| | 7H- | | | | | |
| | pyrrolo[2,3- | | | | | |
| | d]pyrimidine | | | | | |
| 1bd | 4-(4-methylpiperidin- | 4- | 3- | 1 | 8.12 | 386.0 |
| | 1-yl)-2-[3- | methylpiperidine | (methylsulfonyl)aniline | | | |
| | (methylsulfonyl)phenyl]amino- | | | | | |
| | 7H- | | | | | |
| | pyrrolo[2,3- | | | | | |
| | d]pyrimidine | | | | | |
| 1be | 4-(4-methylpiperidin- | 4- | N-(3- | 1 | 8.06 | 401.0 |
| | 1-yl)-2-[3- | methylpiperidine | aminophenyl)methanosulfonamide | | | |
| | (methylsulfonylamino)phenyl]amino- | | | | | |
| | 7H- | | | | | |
| | pyrrolo[2,3- | | | | | |
| | d]pyrimidine | | | | | |
| 1bf | 2-(4- | 4- | 4- | 1 | 9.33 | 333.1 |
| | cyanophenyl)amino- | methylpiperidine | aminobenzonitrile | | | |
| | 4-(4-methylpiperidin- | | | | | |
| | 1-yl)-7H-pyrrolo[2,3- | | | | | |
| | d]pyrimidine | | | | | |
| 1bg | 4-(4-methylpiperidin- | 4- | Reference | 1 | 7.99 | 374.1 |
| | 1-yl)-2-[4-(pirazol-4- | methylpiperidine | example 2 | | | |
| | yl)phenyl]amino-7H- | | | | | |
| | pyrrolo[2,3- | | | | | |
| | d]pyrimidine | | | | | |
| 1bh | 4-(4-methylpiperidin- | 4- | Reference | 1 | 8.22 | 374.1 |
| | 1-yl)-2-[4-(pirazol-3- | methylpiperidine | example 2a | | | |
| | yl)phenyl]amino-7H- | | | | | |
| | pyrrolo[2,3- | | | | | |
| | d]pyrimidine | | | | | |
| 1bi | 2-(3- | 4- | 3-amino- | 4 | 2.23 | 441.2 |
| | aminosulfonylphenyl)amino- | trifluoromethylpiperidine | benzenesulfonamide | | | |
| | 4-(4- | | | | | |
| | trifluoromethylpiperidin- | | | | | |
| | 1-yl)-7H- | | | | | |
| | pyrrolo[2,3- | | | | | |
| | d]pyrimidine | | | | | |
| 1bj | 2-(3- | 3-(n- | 3-amino- | 4 | 2.22 | 459.2 |
| | aminosulfonylphenyl)amino- | butoxycarbonyl)pyrrolidine | benzenesulfonamide | | | |
| | 4-[3-(n- | | | | | |
| | butoxycarbonyl)pyrrolidin- | | | | | |
| | 1-yl]-7H- | | | | | |
| | pyrrolo[2,3- | | | | | |
| | d]pyrimidine | | | | | |
| 1bk | 2-(3- | 4- | 3-amino-N-tert- | 4 | 1.98 | 431.2 |
| | aminosulfonylphenyl)amino- | (ethoxymethyl)piperidine | butylbenzenesulfonamide | | | |
| | 4-[4- | | | | | |
| | (ethoxymethyl)piperidin- | | | | | |
| | 1-yl]-7H- | | | | | |
| | pyrrolo[2,3- | | | | | |
| | d]pyrimidine | | | | | |
| 1bl | 2-(3- | homopiperidine | (3- | 1 | 7.84 | 324.1 |
| | hydroxyphenyl)amino- | | hydroxyphenyl)amine | | | |
| | 4-(homopiperidin- | | | | | |
| | 1-yl)-7H-pyrrolo[2,3- | | | | | |
| | d]pyrimidine | | | | | |
| 1bm | 2-(3- | homopiperidine | (3- | 2 | 2.14 | 365 |
| | acetylaminophenyl)amino- | | acetylaminophenyl)amine | | | |
| | 4- | | | | | |
| | (homopiperidin-1- | | | | | |
| | yl)-7H-pyrrolo[2,3- | | | | | |
| | d]pyrimidine | | | | | |
| 1bn | 2-(3- | 4-(2- | 3-amino- | 4 | 1.52 | 417.2 |
| | aminosulfonylphenyl)amino- | hydroxyethyl)piperidine | benzenesulfonamide | | | |
| | 4-[4-(2- | | | | | |
| | hydroxyethyl)piperidin- | | | | | |
| | 1-yl]-7H- | | | | | |
| | pyrrolo[2,3- | | | | | |
| | d]pyrimidine | | | | | |
| 1bo | 2-(3- | (S)-2- | 3-amino- | 4 | 1.45 | 389.2 |
| | aminosulfonylphenyl)amino- | (hydroxymethyl)pyrrolidine | benzenesulfonamide | | | |
| | 4-[(S)-2- | | | | | |
| | (hydroxymethyl)pyrrolidin- | | | | | |
| | 1-yl]-7H- | | | | | |
| | pyrrolo[2,3- | | | | | |
| | d]pyrimidine | | | | | |
| 1bp | 2-(3- | 4,4- | 3-amino- | 4 | 2.13 | 409.2 |
| | aminosulfonylphenyl)amino- | difluoropiperidine | benzenesulfonamide | | | |
| | 4-(4,4- | | | | | |
| | difluoropiperidin-1- | | | | | |
| | yl)-7H-pyrrolo[2,3- | | | | | |
| | d]pyrimidine | | | | | |
| 1bq | 2-(3- | 3- | 3- | 2 | 1.75 | 393.3 |
| | acetylaminophenyl)amino- | acetylpiperidine | acetylaminophenylamine | | | |
| | 4-(3- | | | | | |
| | acetylpiperidin-1-yl)- | | | | | |
| | 7H-pyrrolo[2,3- | | | | | |
| | d]pyrimidine | | | | | |
| 1br | 2-(3- | (S)-3- | 3-amino- | 4 | 1.42 | 389.2 |
| | aminosulfonylphenyl)amino- | hydroxypiperidine | benzenesulfonamide | | | |
| | 4-[(S)-3- | | | | | |
| | hydroxypiperidin-1- | | | | | |
| | yl]-7H-pyrrolo[2,3- | | | | | |
| | d]pyrimidine | | | | | |
| 1bs | 2-(3- | 4- | 3-amino- | 4 | 1.67 | 403.1 |
| | aminosulfonylphenyl)amino- | methoxypiperidine | benzenesulfonamide | | | |
| | 4-(4- | | | | | |
| | methoxypiperidin-1- | | | | | |
| | yl)-7H-pyrrolo[2,3- | | | | | |
| | d]pyrimidine | | | | | |
| 1bt | 2-(3- | (R)-3- | 3-amino- | 4 | 1.43 | 389.2 |
| | aminosulfonylphenyl)amino- | hydroxypiperidine | benzenesulfonamide | | | |
| | 4-[(R)-3- | | | | | |
| | hydroxypiperidin-1- | | | | | |
| | yl]-7H-pyrrolo[2,3- | | | | | |
| | d]pyrimidine | | | | | |
| 1bu | 2-(3- | (R)-2- | 3-amino- | 4 | 1.45 | 389.2 |
| | aminosulfonylphenyl)amino- | (hydroxymethyl)pyrrolidine | benzenesulfonamide | | | |
| | 4-[(R)-2- | | | | | |
| | (hydroxymethyl)pyrrolidin- | | | | | |
| | 1-yl]-7H- | | | | | |
| | pyrrolo[2,3- | | | | | |
| | d]pyrimidine | | | | | |
| 1bv | 2-(3- | thiazolidine | 3-amino-N-tert- | 2 | 1.72 | 377.2 |
| | aminosulfonylphenyl)amino- | | butylbenzenesulfonamide | | | |
| | 4- | | | | | |
| | (thiazolidin-3-yl)-7H- | | | | | |
| | pyrrolo[2,3- | | | | | |
| | d]pyrimidine | | | | | |
| 1bw | 4-(3-acetylpiperidin- | 3- | 3-amino-N-tert- | 1 | 6.26 | 415 |
| | 1-yl)-2-(3- | acetylpiperidine | butylbenzenesulfonamide | | | |
| | aminosulfonylphenyl)amino- | | | | | |
| | 7H- | | | | | |
| | pyrrolo[2,3- | | | | | |
| | d]pyrimidine | | | | | |
| 1bx | 2-(3- | 4- | 3-amino- | 4 | 1.83 | 391.1 |
| | aminosulfonylphenyl)amino- | fluoropiperidine | benzenesulfonamide | | | |
| | 4-(4- | | | | | |
| | fluoropiperidin-1-yl)- | | | | | |
| | 7H-pyrrolo[2,3- | | | | | |
| | d]pyrimidine | | | | | |
| 1by | 2-(3- | 4- | 3-amino- | 4 | 1.70 | 431 |
| | aminosulfonylphenyl)amino- | hydroxyethylpiperidine | benzenesulfonamide | | | |
| | 4(4- | | | | | |
| | hydroxyethylpiperidin- | | | | | |
| | 1-yl)-7H- | | | | | |
| | pyrrolo[2,3- | | | | | |
| | d]pyrimidine | | | | | |
| 1bz | 2-(3- | 3- | 3-amino- | 2 | 1.46 | 402 |
| | aminosulfonylphenyl)amino- | dimethylaminopyrrolidine | benzenesulfonamide | | | |
| | 4-(3- | | | | | |
| | dimethylaminopyrrolidin- | | | | | |
| | 1-yl)-7H- | | | | | |
| | pyrrolo[2,3- | | | | | |
| | d]pyrimidine | | | | | |
| 1aa | 2-(3- | thiazolidine | 3- | 2 | 1.78 | 355 |
| | acetylaminophenyl)amino- | | aminoacetanilide | | | |
| | 4-(thiazolidin- | | | | | |
| | 3-yl)-7H-pyrrolo[2,3- | | | | | |
| | d]pyrimidine | | | | | |

### Example 2

### 4-(2-Azabicyclo[2.2.1]heptan-2-yl)-2-(3-fluoro-4-methoxyphenyl)amino-7H-pyrrolo[2,3-d]pyrimidine

### a) 4-(2-Azabicyclo[2.2.1]heptan-2-yl)-2-chloro-7H-pyrrolo[2,3-d]pyrimidine

Following a similar procedure to that described in example 1 section a, but using 2-aza-bicyclo[2.2.1]heptane hydrochloride instead of piperidine, the desired compound was obtained (78% yield).

LC-MS (method 2): t_{R}=2.04 min; m/z=249 (MH⁺).

### b) Title Compound

To a solution of the compound obtained in the previous section (100 mg, 0.402 mmol) in tert-butanol (2 mL), K₂CO₃ (167 mg, 1.20 mmol), X-Phos (19 mg, 0.04 mmol), Pd₂(dba)₃ (18 mg, 0.02 mmol) and 3-fluoro-4-methoxyphenylamine (69 mg, 0.48 mmol) were added at room temperature and under Ar-atmosphere. The reaction mixture was heated at 100° C. overnight and the crude product thus obtained was diluted with MeOH and filtered over Celite®. The filtrate was concentrated to dryness and chromatographed over silica gel using CHCl₃/MeOH mixtures of increasing polarity as eluent, to afford 24 mg of the desired compound (17% yield).

LC-MS (method 2): t_{R}=2.53 min; m/z=354 (MH⁺).

Following a similar procedure to that described in example 2, but using in each case the corresponding starting materials, the following compounds were obtained:

| | | Regeant for | Regeant for step | HPLC | t_{R} | |
|---|---|---|---|---|---|---|
| Example | Compound name | step a) | b) | method | (min) | m/z |
| 2a (1) | 2-(3- | 2-benzyl-2,8- | 3-amino- | 4 | 2.22 | 532 |
| | aminosulfonylphenyl)amino- | diaza- | benzenesulfonamide | | | |
| | 4-(2-benzyl- | spiro[4.5]decan- | | | | |
| | 2,8-diaza- | 1-one | | | | |
| | spiro[4.5]decan-1- | hydrochloride | | | | |
| | one-8-yl)-7H- | | | | | |
| | pyrrolo[2,3- | | | | | |
| | d]pyrimidine | | | | | |
| 2b (1) | 2-(3- | 2-methyl-2,8- | 3-amino- | 4 | 1.60 | 456 |
| | aminosulfonylphenyl)amino- | diaza- | benzenesulfonamide | | | |
| | 4-(2-methyl- | spiro[4.5]decan- | | | | |
| | 2,8-diaza- | 1-one | | | | |
| | spiro[4.5]decan-1- | hydrochloride | | | | |
| | one-8-yl)-7H- | | | | | |
| | pyrrolo[2,3- | | | | | |
| | d]pyrimidine | | | | | |
| 2c (1) | 2-[4-(2- | 4- | 4-amino-1-(2- | 4 | 1.47 | 447 |
| | hydroxyethylamino- | hydroxymethylpiperidine | hydroxyethyl)benzene- | | | |
| | sulfonyl)phenyl]amino- | | sulfonamide | | | |
| | 4-(4- | | | | | |
| | hydroxymethylpiperidin- | | | | | |
| | 1-yl)-7H- | | | | | |
| | pyrrolo[2,3- | | | | | |
| | d]pyrimidine | | | | | |
| 2d (1) | 4-(4- | 4- | 4-amino-N-(2- | 4 | 1.28 | 516 |
| | hydroxymethylpiperidin- | hydroxymethylpiperidine | (morpholin-4- | | | |
| | 1-yl)-2-[4-(2- | | yl)ethyl)benzene- | | | |
| | (morpholin-4- | | sulfonamide | | | |
| | yl)ethylaminosulfonyl)- | | | | | |
| | phenyl]amino- | | | | | |
| | 7H- | | | | | |
| | pyrrolo[2,3- | | | | | |
| | d]pyrimidine | | | | | |
| 2e (1) | (S)-2-(3- | (S)-methylpyrrolidine- | 3-amino- | 4 | 1.70 | 417 |
| | aminosulfonylphenyl)amino- | 2- | benzenesulfonamide | | | |
| | 4-[(2- | carboxylate | | | | |
| | methyloxycarbonyl)pyrrolidin- | hydrochloride | | | | |
| | 1-yl]-7H- | | | | | |
| | pyrrolo[2,3- | | | | | |
| | d]pyrimidine | | | | | |
| 2f (1) | 2-(4-(1,1- | 4- | 4-(1,1- | 4 | 1.58 | 457 |
| | dioxothiomorpholin- | hydroxymethylpiperidine | dioxothiomorpholin- | | | |
| | 4-yl)phenyl)amino-4- | | 4-yl)- | | | |
| | (4- | | phenylamine | | | |
| | hydroxymethylpiperidin- | | | | | |
| | 1-yl)-7H- | | | | | |
| | pyrrolo[2,3- | | | | | |
| | d]pyrimidine | | | | | |
| 2g (1) | 2-(3- | 4- | 3- | 4 | 1.48 | 381 |
| | acetylaminophenyl)amino- | hydroxymethylpiperidine | aminoacetanilide | | | |
| | 4-(4- | | | | | |
| | hydroxymethylpiperidin- | | | | | |
| | 1-yl)-7H- | | | | | |
| | pyrrolo[2,3- | | | | | |
| | d]pyrimidine | | | | | |
| 2h (1) | 4-(4- | 4- | 3-(1,3-oxazol-5- | 4 | 1.78 | 391 |
| | hydroxymethylpiperidin- | hydroxymethylpiperidine | yl)phenylamine | | | |
| | 1-yl)-2-(3-(1,3- | | | | | |
| | oxazol-5- | | | | | |
| | yl)phenyl)amino-7H- | | | | | |
| | pyrrolo[2,3- | | | | | |
| | d]pyrimidine | | | | | |
| 2i (1) | 4-(4- | 4- | 3-(1H-imidazol-1- | 4 | 1.17 | 404 |
| | hydroxymethylpiperidin- | hydroxymethylpiperidine | ylmethyl)phenylamine | | | |
| | 1-yl)-2-(3-(1H- | | | | | |
| | imidazol-1- | | | | | |
| | ylmethyl)phenyl)amino- | | | | | |
| | 7H-pyrrolo[2,3- | | | | | |
| | d]pyrimidine | | | | | |
| 2j (1) | (R)-2-(3- | (R)-3- | 3-amino- | 4 | 0.90 | 402 |
| | aminosulfonylphenyl)amino- | dimethylaminopyrrolidine | benzenesulfonamide | | | |
| | 4-(3- | | | | | |
| | dimethylaminopyrrolidin- | | | | | |
| | 1-yl)-7H- | | | | | |
| | pyrrolo[2,3- | | | | | |
| | d]pyrimidine | | | | | |
| 2k | 2-[4-(2- | 4- | 4-amino-1-(2- | 2 | 2.13 | 431 |
| | hydroxyethylaminosulfonyl)- | methylpiperidine | hydroxyethyl)- | | | |
| | phenyl]amino-4-(4- | | benzenesulfonamide | | | |
| | methylpiperidin-1- | | | | | |
| | yl)-7H-pyrrolo[2,3- | | | | | |
| | d]pyrimidine | | | | | |
| 2l | 2-(4-(4- | 4- | 4-(4- | 2 | 2.34 | 406 |
| | methylpiperazin-1- | methylpiperidine | methylpiperazino)- | | | |
| | yl)phenyl)amino-4- | | phenylamine | | | |
| | (4-methylpiperidin-1- | | | | | |
| | yl)-7H-pyrrolo[2,3- | | | | | |
| | d]pyrimidine | | | | | |
| 2m | 4-(4-methylpiperidin- | 4- | 3-pyrrolidin-1- | 2 | 2.50 | 391 |
| | 1-yl)-2-(3-pyrrolidin- | methylpiperidine | ylmethylphenylamine | | | |
| | 1- | | | | | |
| | ylmethylphenyl)amino- | | | | | |
| | 7H-pyrrolo[2,3- | | | | | |
| | d]pyrimidine | | | | | |
| 2n | 4-(2- | 2-aza- | 3-pyrrolidin-1- | 2 | 2.26 | 389 |
| | azabicyclo[2.2.1]heptan- | bicyclo[2.2.1]heptane | ylmethylphenylamine | | | |
| | 2-yl)-2-(3- | hydrochloride | | | | |
| | pyrrolidin-1- | | | | | |
| | ylmethylphenyl)amino- | | | | | |
| | 7H-pyrrolo[2,3- | | | | | |
| | d]pyrimidine | | | | | |
| 2o (1) | 4-(4- | 4- | 4-(4- | 4 | 1.13 | 422 |
| | hydroxymethylpiperidin- | hydroxymethylpiperidine | methylpiperazino)- | | | |
| | 1-yl)-2-(4-(4- | | phenylamine | | | |
| | methylpiperazin-1- | | | | | |
| | yl)phenyl)amino-7H- | | | | | |
| | pyrrolo[2,3- | | | | | |
| | d]pyrimidine | | | | | |
| 2p (1) | 2-(3- | 4- | 3- | 4 | 1.58 | 368 |
| | hydroxyethylphenyl)amino- | hydroxymethylpiperidine | hydroxyethylphenylamine | | | |
| | 4-(4- | | | | | |
| | hydroxymethylpiperidin- | | | | | |
| | 1-yl)-7H- | | | | | |
| | pyrrolo[2,3- | | | | | |
| | d]pyrimidine | | | | | |
| 2q (1) | 2-(3- | 4-[(2-methyl- | 3-amino- | 4 | 1.18 | 467 |
| | aminosulfonylphenyl)amino- | 1H-imidazol-1- | benzenesulfonamide | | | |
| | 4-(4-[(2- | yl)methyl]piperidine | | | | |
| | methyl-1H-imidazol- | | | | | |
| | 1- | | | | | |
| | yl)methyl]piperidin- | | | | | |
| | 1-yl)-7H-pyrrolo[2,3- | | | | | |
| | d]pyrimidine | | | | | |
| 2r (1) | 4-(4- | 4- | 3-amino-N- | 4 | 1.60 | 417 |
| | hydroxymethylpiperidin- | hydroxymethylpiperidine | methylbenzenesulfonamide | | | |
| | 1-yl)-2-[3-(N- | | | | | |
| | methylaminosulfonyl)- | | | | | |
| | phenyl]amino- | | | | | |
| | 7H- | | | | | |
| | pyrrolo[2,3- | | | | | |
| | d]pyrimidine | | | | | |
| 2s (1) | 4-(4-(3- | 4-(3- | 3-pyrrolidin-1- | 4 | 1.45 | 435 |
| | hydroxypropylpiperidin- | hydroxypropyl)piperidine | ylmethylphenylamine | | | |
| | 1-yl)-2-(3- | | | | | |
| | pyrrolidin-1- | | | | | |
| | ylmethylphenyl)amino- | | | | | |
| | 7H-pyrrolo[2,3- | | | | | |
| | d]pyrimidine | | | | | |
| 2t (1) | (R)-2-(3- | (R)-2- | 3-amino- | 4 | 1.83 | 373 |
| | aminosulfonylphenyl)amino- | methylpyrrolidine | benzenesulfonamide | | | |
| | 4-(2- | | | | | |
| | methylpyrrolidin-1- | | | | | |
| | yl)-7H-pyrrolo[2,3- | | | | | |
| | d]pyrimidine | | | | | |
| 2u (1) | (S)-2-(3- | (S)-2-methylpiperidine | 3-amino- | 4 | 2.03 | 387 |
| | aminosulfonylphenyl)amino- | | benzenesulfonamide | | | |
| | 4-[(2- | | | | | |
| | methyl)piperidin-1- | | | | | |
| | yl]-7H-pyrrolo[2,3- | | | | | |
| | d]pyrimidine | | | | | |
| 2v | 2-(3- | 1,4- | 3-amino- | 2 | 1.62 | 414 |
| | aminosulfonylphenyl)amino- | diazabicyclo[4.3.0]nonane | benzenesulfonamide | | | |
| | 4-(1,4- | | | | | |
| | diazabicyclo[4.3.0]nonan- | | | | | |
| | 4-yl)-7H- | | | | | |
| | pyrrolo[2,3- | | | | | |
| | d]pyrimidine | | | | | |
| 2w (1) | 2-(3- | 3,3-dimethylpiperidine | 3-amino- | 4 | 2.27 | 401 |
| | aminosulfonylphenyl)amino- | | benzenesulfonamide | | | |
| | 4-[(3,3- | | | | | |
| | dimethyl)piperidin-1- | | | | | |
| | yl]-7H-pyrrolo[2,3- | | | | | |
| | d]pyrimidine | | | | | |
| 2x (1) | (S)-4-(2- | (S)-2- | 4-pyrrolidin-1- | 4 | 1.25 | 393 |
| | hydroxymethylpyrrolidin- | hydroxymethylpyrrolidine | ylmethylphenylamine | | | |
| | 1-yl)-2-(4- | | | | | |
| | pyrrolidin-1- | | | | | |
| | ylmethylphenyl)amino- | | | | | |
| | 7H-pyrrolo[2,3- | | | | | |
| | d]pyrimidine | | | | | |
| 2y (1) | (R)-2-(3- | (R)-2-methylpiperidine | 3-amino- | 4 | 2.12 | 387 |
| | aminosulfonylphenyl)amino- | | benzenesulfonamide | | | |
| | 4-([2- | | | | | |
| | methyl]piperidin-1- | | | | | |
| | yl)-7H-pyrrolo[2,3- | | | | | |
| | d]pyrimidine | | | | | |
| 2z (1) | (S)-2-[(3- | (S)-3- | N-(3- | 4 | 1.75 | 393 |
| | cyclopropylcarbonyl- | hydroxypiperidine | aminophenyl)cyclo- | | | |
| | amino)phenyl]amino- | | propanecarboxamide | | | |
| | 4-(3- | | | | | |
| | hydroxypiperidin-1- | | | | | |
| | yl)-7H-pyrrolo[2,3- | | | | | |
| | d]pyrimidine | | | | | |
| 2aa (1) | (S)-4-(3- | (S)-3- | 3-pyrrolidin-1- | 4 | 1.18 | 393 |
| | hydroxypiperidin-1- | hydroxypiperidine | ylmethylphenylamine | | | |
| | yl)-2-(3-pyrrolidin-1- | | | | | |
| | ylmethylphenyl)amino- | | | | | |
| | 7H-pyrrolo[2,3- | | | | | |
| | d]pyrimidine | | | | | |
| 2ab | 4-(homopiperidin-1- | homopiperidine | 3-pyrrolidin-1- | 2 | 2.42 | 391 |
| | yl)-2-(3-pyrrolidin-1- | | ylmethylphenylamine | | | |
| | ylmethylphenyl)amino- | | | | | |
| | 7H-pyrrolo[2,3- | | | | | |
| | d]pyrimidine | | | | | |
| 2ac | 4-(homopiperidin-1- | homopiperidine | 4-(4- | 2 | 2.27 | 406 |
| | yl)-2-[4-(4- | | methylpiperazin- | | | |
| | methylpiperazin-1- | | 1-ylphenylamine | | | |
| | yl)phenyl]amino-7H- | | | | | |
| | pyrrolo[2,3- | | | | | |
| | d]pyrimidine | | | | | |
| 2ad (1) | 2-[3- | 4- | (3-amino-phenyl)- | 4 | 1.47 | 417 |
| | (aminosulfonylmethyl)- | hydroxymethylpiperidine | methanesulfonamide | | | |
| | phenyl]amino- | | | | | |
| | 4-(4- | | | | | |
| | hydroxymethylpiperidin- | | | | | |
| | 1-yl)-7H- | | | | | |
| | pyrrolo[2,3- | | | | | |
| | d]pyrimidine | | | | | |
| 2ae | 4-(2- | 2-aza- | 4- | 2 | 2.09 | 350 |
| | azabicyclo[2.2.1]heptan- | bicyclo[2.2.1]heptane | hydroxyethylphenylamine | | | |
| | 2-yl)-2-(4- | hydrochloride | | | | |
| | hydroxyethylphenyl)amino- | | | | | |
| | 7H- | | | | | |
| | pyrrolo[2,3- | | | | | |
| | d]pyrimidine | | | | | |
| 2af | 4-(2- | 2-aza- | 3- | 2 | 2.12 | 350 |
| | azabicyclo[2.2.1]heptan- | bicyclo[2.2.1]heptane | hydroxyethylphenylamine | | | |
| | 2-yl)-2-(3- | hydrochloride | | | | |
| | hydroxyethylphenyl)amino- | | | | | |
| | 7H- | | | | | |
| | pyrrolo[2,3- | | | | | |
| | d]pyrimidine | | | | | |
| 2ag (1) | (R)-4-(3-(N,N- | (R)-3-(N,N- | 3-amino-N- | 4 | 1.03 | 416 |
| | dimethylamino)pyrrolidin- | dimethylamino)pyrrolidine | methylbenzenesulfonamide | | | |
| | 1-yl)-2-(3- | | | | | |
| | methylamino- | | | | | |
| | sulfonylphenyl)amino- | | | | | |
| | 7H-pyrrolo[2,3- | | | | | |
| | d]pyrimidine | | | | | |
| 2ah (1) | (R)-2-(3- | (R)-3-(N,N- | 3- | 4 | 0.9 | 380 |
| | acetylaminophenyl)amino- | dimethylamino)pyrrolidine | aminoacetanilide | | | |
| | 4-(3-(N,N- | | | | | |
| | dimethylamino)pyrrolidin- | | | | | |
| | 1-yl)-7H- | | | | | |
| | pyrrolo[2,3- | | | | | |
| | d]pyrimidine | | | | | |
| 2ai (1) | (R)-2-[3- | (R)-3-(N,N- | 3- | 4 | 1.47 | 414 |
| | (phenylamino)phenyl]amino- | dimethylamino)pyrrolidine | (phenylamino)phenylamine | | | |
| | 4-(3-(N,N- | | | | | |
| | dimethylamino)pyrrolidin- | | | | | |
| | 1-yl)-7H- | | | | | |
| | pyrrolo[2,3- | | | | | |
| | d]pyrimidine | | | | | |
| 2aj (1) | (R)-4-(3-(N,N- | (R)-3-(N,N- | [4-(4- | 4 | 0.92 | 408 |
| | dimethylamino)pyrrolidin- | dimethylamino)pyrrolidine | morpholino)phenyl]amine | | | |
| | 1-yl)-2-[4- | | | | | |
| | (morpholin-4- | | | | | |
| | yl)phenyl]amino-7H- | | | | | |
| | pyrrolo[2,3- | | | | | |
| | d]pyrimidine | | | | | |
| 2ak (1) | (R)-4-(3-(N,N- | (R)-3-(N,N- | 3-fluoro-4- | 4 | 1.03 | 371 |
| | dimethylamino)pyrrolidin- | dimethylamino)pyrrolidine | methoxyphenylamine | | | |
| | 1-yl)-2-(3- | | | | | |
| | fluoro-4- | | | | | |
| | methoxyphenyl)amino- | | | | | |
| | 7H-pyrrolo[2,3- | | | | | |
| | d]pyrimidine | | | | | |
| 2al | 4-(2- | 2-aza- | 4-(morpholin-4- | 2 | 2.23 | 405 |
| | azabicyclo[2.2.1]heptan- | bicyclo[2.2.1]heptane | yl)methylphenylamine | | | |
| | 2-yl)-2-[(4- | hydrochloride | | | | |
| | morpholin-4- | | | | | |
| | yl)methylphenyl]amino- | | | | | |
| | 7H-pyrrolo[2,3- | | | | | |
| | d]pyrimidine | | | | | |
| 2am | 4-(2- | 2-aza- | 4-(1- | 2 | 1.99 | 418 |
| | azabicyclo[2.2.1]heptan- | bicyclo[2.2.1]heptane | methylpiperazin- | | | |
| | 2-yl)-2-(4-(1- | hydrochloride | 4- | | | |
| | methylpiperazin-4- | | yl)methylphenylamine | | | |
| | yl)methylphenyl)amino- | | | | | |
| | 7H-pyrrolo[2,3- | | | | | |
| | d]pyrimidine | | | | | |
| 2an | 4-(2- | 2-aza- | Reference | 2 | 2.11 | 377 |
| | azabicyclo[2.2.1]heptan- | bicyclo[2.2.1]heptane | example 3 | | | |
| | 2-yl)-2-[3-(N- | hydrochloride | | | | |
| | methylacetamido)phenyl]amino- | | | | | |
| | 7H- | | | | | |
| | pyrrolo[2,3- | | | | | |
| | d]pyrimidine | | | | | |
| 2ao | 4-(2- | 2-aza- | Reference | 2 | 2.32 | 404 |
| | azabicyclo[2.2.1]heptan- | bicyclo[2.2.1]heptane | example 3a | | | |
| | 2-yl)-2-[3-(N- | hydrochloride | | | | |
| | methylcyclopropane- | | | | | |
| | carbonylamino)phenyl]amino- | | | | | |
| | 7H- | | | | | |
| | pyrrolo[2,3- | | | | | |
| | d]pyrimidine | | | | | |
| 2ap (1), (2) | 4-(4- | 4- | 4-(1-tert- | 4 | 1.18 | 407 |
| | hydroxymethylpiperidin- | hydroxymethylpiperidine | butoxycarbonylpiperidin- | | | |
| | 1-yl)-2-[4- | | 4-yl)- | | | |
| | (piperidin-4-yl)- | | phenylamine | | | |
| | phenyl]amino-7H- | | | | | |
| | pyrrolo[2,3- | | | | | |
| | d]pyrimidine | | | | | |
| 2aq | 2-(3- | Reference | 3-amino- | 2 | 1.51 | 453 |
| | aminosulfonylphenyl)amino- | example 4 | benzenesulfonamide | | | |
| | 4[4- | | | | | |
| | (imidazol-1- | | | | | |
| | ylmethyl)piperidin-1- | | | | | |
| | yl]-7H-pyrrolo[2,3- | | | | | |
| | d]pyrimidine | | | | | |
| 2ar | 2-(3- | Reference | 3-amino- | 1 | 6.48 | 501 |
| | aminosulfonylphenyl)amino- | example 5 | benzenesulfonamide | | | |
| | 4-[4-[(N′-tert- | | | | | |
| | butylureido)methyl]piperidin- | | | | | |
| | 1-yl]-7H- | | | | | |
| | pyrrolo[2,3- | | | | | |
| | d]pyrimidine | | | | | |
| 2as | 4-(3- | 3- | 3-amino- | 2 | 1.24 | 416 |
| | acetamidopyrrolidin- | acetamidopyrrolidine | benzenesulfonamide | | | |
| | 1-yl)-2-(3- | | | | | |
| | aminosulfonylphenyl)amino- | | | | | |
| | 7H- | | | | | |
| | pyrrolo[2,3- | | | | | |
| | d]pyrimidine | | | | | |
| 2at | 2-(3- | 3,3- | 3-amino- | 2 | 1.57 | 417 |
| | aminosulfonylphenyl)amino- | dimethylpiperidin- | benzenesulfonamide | | | |
| | 4-(3,3- | 4-ol (3) | | | | |
| | dimethyl-4- | | | | | |
| | hydroxypiperidin-1- | | | | | |
| | yl)-7H-pyrrolo[2,3- | | | | | |
| | d]pyrimidine | | | | | |
| 2au | 4-(1′-acetyl-[4,4′]- | 1-(4,4′- | 3-amino- | 4 | 1.85 | 498 |
| | bipiperidin-1-yl)-2- | bipiperidin-1- | benzenesulfonamide | | | |
| | (3- | yl)ethanone | | | | |
| | aminosulfonylphenyl)amino- | | | | | |
| | 7H- | | | | | |
| | pyrrolo[2,3- | | | | | |
| | d]pyrimidine | | | | | |
| 2av | 4-(2- | 2-aza- | 4-(4- | 2 | 2.07 | 404 |
| | azabicyclo[2.2.1]heptan- | bicyclo[2.2.1]heptane | methylpiperazin- | | | |
| | 2-yl)-2-(4-[4- | hydrochloride | 1-ylphenylamine | | | |
| | methylpiperazin-1- | | | | | |
| | yl]phenyl)amino-7H- | | | | | |
| | pyrrolo[2,3- | | | | | |
| | d]pyrimidine | | | | | |
| 2aw | 4-(2- | 2-aza- | 4-(piperazin-1- | 2 | 1.78 | 390 |
| | azabicyclo[2.2.1]heptan- | bicyclo[2.2.1]heptane | yl)phenylamine | | | |
| | 2-yl)-2-[4- | hydrochloride | | | | |
| | (piperazin-1- | | | | | |
| | yl)phenyl]amino-7H- | | | | | |
| | pyrrolo[2,3- | | | | | |
| | d]pyrimidine | | | | | |
| 2ax | 4-(2- | 2-aza- | 4-(2-pyrrolidin-1- | 2 | 2.11 | 419 |
| | azabicyclo[2.2.1]heptan- | bicyclo[2.2.1]heptane | yl)ethoxyaniline | | | |
| | 2-yl)-2-[4-(2- | hydrochloride | | | | |
| | (pyrrolidin-1- | | | | | |
| | yl)ethoxyphenyl]amino- | | | | | |
| | 7H-pyrrolo[2,3- | | | | | |
| | d]pyrimidine | | | | | |
| 2ay | (S)-2-(3- | (S)-3-(tert- | 3-amino- | 2 | 1.99 | 488 |
| | aminosulfonylphenyl)amino- | butoxycarbonylamino- | benzenesulfonamide | | | |
| | 4-[3-(tert- | methyl)pyrrolidine | | | | |
| | butoxycarbonylamino- | | | | | |
| | methyl)pyrrolidin- | | | | | |
| | 1- | | | | | |
| | yl]-7H-pyrrolo[2,3- | | | | | |
| | d]pyrimidine | | | | | |
| 2az | trans-2-(3- | trans-4- | 3-amino- | 2 | 1.22 | 418 |
| | aminosulfonylphenyl)amino- | dimethylaminopyrrolidin- | benzenesulfonamide | | | |
| | 4-(4- | 3-ol (4) | | | | |
| | dimethylamino-3- | | | | | |
| | hydroxypyrrolidin-1- | | | | | |
| | yl)-7H-pyrrolo[2,3- | | | | | |
| | d]pyrimidine | | | | | |
| 2ba | (R)-2-(3- | (R)-3- | 3-amino- | 4 | 1.85 | 373 |
| | aminosulfonylphenyl)amino- | methylpyrrolidine | benzenesulfonamide | | | |
| | 4-(3- | | | | | |
| | methylpyrrolidin-1- | | | | | |
| | yl)-7H-pyrrolo[2,3- | | | | | |
| | d]pyrimidine | | | | | |
| 2bb | (R)-2-(3- | 3-(R)-(N-tert- | 3-amino- | 2 | 2.14 | 488 |
| | aminosulfonylphenyl)amino- | butoxycarbonyl- | benzenesulfonamide | | | |
| | 4-[3-(N-tert- | N- | | | | |
| | butoxycarbonyl-N- | methylamino)pyrrolidine | | | | |
| | methylamino)pyrrolidin1- | | | | | |
| | yl]-7H- | | | | | |
| | pyrrolo[2,3- | | | | | |
| | d]pyrimidine | | | | | |
| 2bc | (R)-4-[3-(N-tert- | 3-(R)-(N-tert- | 3-fluoro-4- | 4 | 2.50 | 457 |
| | butoxycarbonyl-N- | butoxycarbonyl- | methoxyphenylamine | | | |
| | methylamino)pyrrolidin1- | N- | | | | |
| | yl]-2-(3-fluoro- | methylamino)pyrrolidine | | | | |
| | 4- | | | | | |
| | methoxyphenyl)amino- | | | | | |
| | 7H-pyrrolo[2,3- | | | | | |
| | d]pyrimidine | | | | | |
| 2bd | (R)-2-(3- | 3-(R)-(N-tert- | 3-amino- | 2 | 1.98 | 474 |
| | aminosulfonylphenyl)amino- | butoxycarbonylamino)- | benzenesulfonamide | | | |
| | 4-[3-(N-tert- | pyrrolidine | | | | |
| | butoxycarbonylamino)pyrrolidin1- | | | | | |
| | yl]-7H- | | | | | |
| | pyrrolo[2,3- | | | | | |
| | d]pyrimidine | | | | | |
| 2be | (R)-4-(3- | 3-(R)- | 3-amino- | 2 | 1.27 | 416 |
| | acetamidopyrrolidin1- | acetamidopyrrolidine | benzensulfonamide | | | |
| | yl)-2-(3- | | | | | |
| | aminosulfonylphenyl)amino- | | | | | |
| | 7H- | | | | | |
| | pyrrolo[2,3- | | | | | |
| | d]pyrimidine | | | | | |
| 2bf | 2-(3- | Reference | 3-amino- | 2 | 1.46 | 403 |
| | aminosulfonylphenyl)amino- | example 6 | benzenesulfonamide | | | |
| | 4-(4- | | | | | |
| | hydroxy-2- | | | | | |
| | methylpiperidin-1- | | | | | |
| | yl)-7H-pyrrolo[2,3- | | | | | |
| | d]pyrimidine | | | | | |
| 2bg | trans-2-(3- | trans-4- | 3-amino- | 5 | 3.13 | 403 |
| | aminosulfonylphenyl)amino- | methylpiperidin- | benzenesulfonamide | | | |
| | 4-(3- | 3-ol (5) | | | | |
| | hydroxy-4- | | | | | |
| | methylpiperidin-1- | | | | | |
| | yl)-7H-pyrrolo[2,3- | | | | | |
| | d]pyrimidine | | | | | |
| 2bh | cis-2-(3- | cis-2- | 3-amino- | 5 | 3.04 | 403 |
| | aminosulfonylphenyl)amino- | methylpiperidin- | benzenesulfonamide | | | |
| | 4-(3- | 3-ol (6) | | | | |
| | hydroxy-2- | | | | | |
| | methylpiperidin-1- | | | | | |
| | yl)-7H-pyrrolo[2,3- | | | | | |
| | d]pyrimidine | | | | | |
| 2bi | cis-2-(3- | cis-6- | 3-amino- | 5 | 3.09 | 403 |
| | aminosulfonylphenyl)amino- | methylpiperidin- | benzenesulfonamide | | | |
| | 4-(3- | 3-ol (6) | | | | |
| | hydroxy-6- | | | | | |
| | methylpiperidin-1- | | | | | |
| | yl)-7H-pyrrolo[2,3- | | | | | |
| | d]pyrimidine | | | | | |
| 2bj | cis-2-(3- | cis-5- | 3-amino- | 5 | 3.22 | 403 |
| | aminosulfonylphenyl)amino- | methylpiperidin- | benzenesulfonamide | | | |
| | 4-(3- | 3-ol (6) | | | | |
| | hydroxy-5- | | | | | |
| | methylpiperidin-1- | | | | | |
| | yl)-7H-pyrrolo[2,3- | | | | | |
| | d]pyrimidine | | | | | |
| 2bk | (6S,8R)-2-(3- | Reference | 3-amino- | 2 | 1.20 | 555.6 |
| | aminosulfonylphenyl)amino- | example 7 | benzenesulfonamide | | | |
| | 4-(8- | | | | | |
| | hydroxy-1,4- | | | | | |
| | diazabicyclo[4.3.0]nonan- | | | | | |
| | 4-yl)-7H- | | | | | |
| | pyrrolo[2,3- | | | | | |
| | d]pyrimidine | | | | | |
| 2bl | (S)-2-(3- | Reference | 3-amino- | 2 | 1.62 | 414.4 |
| | aminosulfonylphenyl)amino- | example 7a | benzenesulfonamide | | | |
| | 4-(1,4- | | | | | |
| | diazabicyclo[4.3.0]nonan- | | | | | |
| | 4-yl)-7H- | | | | | |
| | pyrrolo[2,3- | | | | | |
| | d]pyrimidine | | | | | |
| 2bm | (S)-2-(4- | Reference | 4- | 2 | 1.70 | 379.5 |
| | hydroxyethylphenyl)amino- | example 7a | hydroxyethylphenylamine | | | |
| | 4-(1,4- | | | | | |
| | diazabicyclo[4.3.0]nonan- | | | | | |
| | 4-yl)-7H- | | | | | |
| | pyrrolo[2,3- | | | | | |
| | d]pyrimidine | | | | | |
| 2bn | (6S,3S)-2-(3- | Reference | 3-amino- | 2 | 1.88 | 428.4 |
| | aminosulfonylphenyl)amino- | example 7b | benzenesulfonamide | | | |
| | 4-(3-methyl- | | | | | |
| | 1,4- | | | | | |
| | diazabicyclo[4.3.0]nonan- | | | | | |
| | 4-yl)-7H- | | | | | |
| | pyrrolo[2,3- | | | | | |
| | d]pyrimidine | | | | | |
| 2bo | 2-(3- | 2- | 3-amino- | 4 | 2.05 | 387.3 |
| | aminosulfonylphenyl)amino- | methylpiperidine | benzenesulfonamide | | | |
| | 4-[(2- | | | | | |
| | methyl)piperidin-1- | | | | | |
| | yl]-7H-pyrrolo[2,3- | | | | | |
| | d]pyrimidine | | | | | |
| 2bp | 2-(3- | 2- | 3-amino- | 4 | 1.62 | 403.2 |
| | aminosulfonylphenyl)amino- | (hydroxymethyl)piperidine | benzenesulfonamide | | | |
| | 4-[(2- | | | | | |
| | hydroxymethyl)piperidin- | | | | | |
| | 1-yl]-7H- | | | | | |
| | pyrrolo[2,3- | | | | | |
| | d]pyrimidine | | | | | |
| 2bq | 4-[(4- | 4- | 4-amino-N-(2- | 4 | 1.67 | 461.3 |
| | hydroxymethyl)piperidin- | (hydroxymethyl)piperidine | methoxyethyl)- | | | |
| | 1-yl]-2-[4- | benzenesulfonamide | | | | |
| | (methoxyethyl)amino- | | | | | |
| | sulfonylphenyl]amino- | | | | | |
| | 7H-pyrrolo[2,3- | | | | | |
| | d]pyrimidine | | | | | |
| 2br | 4-[(4- | 4- | 3-pyrrolidin-1- | 4 | 1.18 | 407.3 |
| | hydroxymethyl)piperidin- | (hydroxymethyl)piperidine | ylmethylphenylamine | | | |
| | 1-yl]-2-(3- | | | | | |
| | pyrrolidin-1- | | | | | |
| | ylmethylphenyl)amino- | | | | | |
| | 7H-pyrrolo[2,3- | | | | | |
| | d]pyrimidine | | | | | |

(1) First step using 1,4-dioxane instead of ethanol
(2) An additional deprotection step was necessary: over a solution of the product obtained, 4M dioxane/HCl_{(g)} (2 mL) were added to afford the desired product.
(3) Described in WO/2005/026145
(4) Described in WO/2007/146759
(5) Described in WO/2001/087866
(6) Described in WO/2007/122103

### Example 3

### (R)-2-(3-Aminosulfonylphenyl)amino-4-(3-(1-methylureido)pyrrolidin1-yl)-7H-pyrrolo[2,3-d]pyrimidine

### a) (R)-2-(3-Aminosulfonylphenyl)amino-4-(3-(N-methylamino)pyrrolidin1-yl)-7H-pyrrolo[2,3-d]pyrimidine

A mixture of the compound obtained in example 2bb (390 mg, 0.8 mmol), 4M dioxane/HCl_{(g)} (7 mL), and methanol (3 mL) was stirred under Ar-atmosphere for 2 h at room temperature. The resulting mixture was concentrated to dryness and the residue thus obtained was partitioned between 0.2 N NaHCO₃ and CHCl₃. The phases were separated and the combined organic phases were dried over Na₂SO₄ and concentrated to dryness to afford 225 mg of the desired product.

LC-MS (method 2): t_{R}=1.27 min; m/z=388 (MH⁺).

### b) Title Compound

To a solution of the compound obtained in the previous section (40 mg, 0.1 mmol) in DMF (1 mL), trimethylsilyl isocyanate (14 mg, 0.12 mmol) was added under Ar-atmosphere and the mixture was stirred at room temperature overnight. The resulting solution was concentrated to dryness, diluted with EtOAc and washed twice with NH₄Cl saturated aqueous solution. The combined organic phases were dried over Na₂SO₄ and concentrated to dryness. The crude product thus obtained was chromatographed over silica gel using EtOAc/MeOH/NH₃ mixtures of increasing polarity as eluent, to afford 18 mg of the desired compound (43% yield).

LC-MS (method 2): t_{R}=1.23 min; m/z=431 (MH⁺).

Following a similar procedure to that described in example 3, but using the corresponding starting material, the following compounds were obtained:

| | | Reagent for step | Reagent for step | HPLC | t_{R} | |
|---|---|---|---|---|---|---|
| Example | Compound name | a) | b) | method | (min) | m/z |
| 3a | (R)-2-(3- | Example 2bb | methanesulfonyl | 2 | 1.55 | 466 |
| | aminosulfonylphenyl)amino- | | chloride (1) | | | |
| | 4-[3-(N- | | | | | |
| | methylmethanesulfonylamino)pyrrolidin1- | | | | | |
| | yl]-7H-pyrrolo[2,3- | | | | | |
| | d]pyrimidine | | | | | |
| 3b | (R)-2-(3- | Example 2bh | phenyl | 2 | 1.91 | 494 |
| | aminosulfonylphenyl)amino- | | chloroformate (1) | | | |
| | 4-[3-(N- | | | | | |
| | phenoxycarbonylamino)pyrrolidin1- | | | | | |
| | yl]- | | | | | |
| | 7H-pyrrolo[2,3- | | | | | |
| | d]pyrimidine | | | | | |
| 3c | (R)-2-(3- | Example 2bh | methanesulfonyl | 2 | 1.38 | 452 |
| | aminosulfonylphenyl)amino- | | chloride | | | |
| | 4-[3-(N- | | | | | |
| | methanesulfonylamino)pyrrolidin1- | | | | | |
| | yl]- | | | | | |
| | 7H-pyrrolo[2,3- | | | | | |
| | d]pyrimidine | | | | | |
| 3d | (R)-2-(3- | Example 2bh | trimethylsilyl | 2 | 1.15 | 417 |
| | aminosulfonylphenyl)amino- | | isocyanate | | | |
| | 4-(3- | | | | | |
| | ureidopyrrolidin-1- | | | | | |
| | yl)-7H-pyrrolo[2,3- | | | | | |
| | d]pyrimidine | | | | | |

(1) Using pyridine instead of DMF as a solvent

### Example 4

### (R)-2-(3-Aminosulfonylphenyl)amino-4-(3-(3-methylureido)pyrrolidin1-yl)-7H-pyrrolo[2,3-d]pyrimidine

To a solution of the compound obtained in example 3c (27 mg, 0.05 mmol) in pyridine (2 mL), a 2 M solution of methylamine in THF (0.27 mL, 0.54 mmol) was added under Ar-atmosphere. The resulting mixture was heated at 100° C. overnight and concentrated to dryness. The crude product thus obtained was chromatographed over silica gel using CHCl₃/MeOH/NH₃ mixtures of increasing polarity as eluent, to afford the desired compound in quantitative yield.

LC-MS (method 2): t_{R}=1.24 min; m/z=431 (MH⁺).

Following a similar procedure to that described in example 4, but using the corresponding starting material, the following compound were obtained:

| Ex- | | | | | |
|---|---|---|---|---|---|
| am- | | Starting | HPLC | t_{R} | |
| ple | Compound name | material | method | (min) | m/z |
| 4a | (R)-2-(3- | 2,2,2- | 2 | 1.58 | 499 |
| | aminosulfonylphenyl)amino- | trifluoro- | | | |
| | 4-{3-[3-(2,2,2- | ethyl- | | | |
| | trifluoro)ethylureido]- | amine | | | |
| | pyrrolidin1- | | | | |
| | yl}-7H-pyrrolo[2,3- | | | | |
| | d]pyrimidine | | | | |
| 4b | (R)-2-(3- | diethyl- | 2 | 1.65 | 473 |
| | aminosulfonylphenyl)amino- | amine | | | |
| | 4-(3-((3,3- | | | | |
| | diethylureido)pyrrolidin1-yl)- | | | | |
| | 7H-pyrrolo[2,3-d]pyrimidine | | | | |

### Example 5

### (S)-2-(3-Aminosulfonylphenyl)amino-4-(3-aminomethylpyrrolidin-1-yl)-7H-pyrrolo[2,3-d]pyrimidine

Following a similar procedure to that described in example 3 section a, but using 2ay instead of example 2bb, the product was obtained (44% yield).

LC-MS (method 2): t_{R}=1.08 min; m/z=388.3 (MH⁺).

### Example 6

### 2-(3-Acetylaminosulfonylphenyl)amino-4-(4-methylpiperidin-1-yl)-7H-pyrrolo[2,3-d]pyrimidine

A mixture of the compound obtained in example 1b (34 mg, 0.088 mmol), acetic anhydride (0.025 mL, 0.264 mmol) and triethylamine (0.011 mL, 0.088 mmol) in CHCl₃ (2 mL) was stirred at room temperature overnight. The resulting solution was diluted with CHCl₃ and washed with water and brine. The combined organic phases were dried over Na₂SO₄ and concentrated to dryness. The crude product obtained was chromatographed over silica gel using CH₂Cl₂/MeOH mixtures of increasing polarity as eluent, to afford 25 mg of the desired compound (55% yield).

LC-MS (method 2): t_{R}=1.71 min; m/z=429 (MH⁺).

Following a similar procedure to that described in example 6, but using the corresponding starting material, the following compound is obtained:

| | | Starting | HPLC | t_{R} | |
|---|---|---|---|---|---|
| Example | Compound name | material | method | (min) | m/z |
| 6a | 4-(4-methylpiperidin- | propionyl | 2 | 1.75 | 443.5 |
| | 1-yl)-2-(3- | chloride | | | |
| | propionylamino- | | | | |
| | sulfonylphenyl)amino- | | | | |
| | 7H-pyrrolo[2,3- | | | | |
| | d]pyrimidine | | | | |

### Example 7

### 2-(3-Acetylaminosulfonylphenyl)amino-4-(4-methylpiperidin-1-yl)-7H-pyrrolo[2,3-d]pyrimidine sodium salt

To a solution of example 6 (16 mg, 0.039 mmol) in EtOH (1.5 mL), a 0.05 M aqueous solution of NaOH in EtOH (0.78 mL) was added. The mixture was stirred at room temperature for 30 min and concentrated to dryness to afford 18 mg of the desired product (100% yield).

LC-MS (method 2): t_{R}=1.71 min; m/z=429 (MH⁺).

### Example 8

### (2S,4S)-2-(3-Aminosulfonylphenyl)amino-4-(2-hydroxymethyl-4-hydroxypyrrolidin-1-yl)-7H-pyrrolo[2,3-d]pyrimidine

### a) (2S,4S)-2-Chloro-4-(2-methoxycarbonyl-4-hydroxypyrrolidin-1-yl)-7H-pyrrolo[2,3-d]pyrimidine

Following a similar procedure to that described in example 1 section a, but using (2S,4S)-methyl-4-hydroxy-2-pyrrolidinecarboxylate instead of piperidine the desired product was obtained (61%).

LC-MS (method 2): t_{R}=1.19 min; m/z=297 (MH⁺).

### b) (2S,4S)-2-(3-Amino-N-tert-butylsulfonylphenyl)amino-4-(2-methoxycarbonyl-4-hydroxypyrrolidin-1-yl)-7H-pyrrolo[2,3-d]pyrimidine

Following a similar procedure to that described in example 2 section b, but using 3-amino-N-tert-butylbenzenesulfonamide instead of 3-fluoro-4-methoxyphenylamine, the desired product was obtained (52% yield).

LC-MS (method 2): t_{R}=1.78 min; m/z=489.3 (MH⁺).

### c) (2S,4S)-2-(3-Amino-N-tert-butylsulfonylphenyl)amino-4-(2-hydroxymethyl-4-hydroxypyrrolidin-1-yl)-7H-pyrrolo[2,3-d]pyrimidine

A solution of the compound obtained in the previous section (357 mg, 0.731 mmol) in THF (8 mL) was added to a suspension of LiAlH₄ (56 mg, 1.462 mmol) in THF (4 mL) under Ar-atmosphere. The mixture was refluxed overnight, cooled and diluted with CH₂Cl₂ (0.766 mL) The resulting mixture was treated with a saturated solution of sodium tartrate (0.076 mL). The organic phase was dried over Na₂SO₄ and concentrated to dryness. The crude product thus obtained was chromatographed over silica gel using hexane/EtOAc mixtures of increasing polarity as eluent, to afford 79 mg of the desired compound (35% yield).

LC-MS (method 2): t_{R}=1.67 min; m/z=461 (MH⁺).

### d) Title Compound

A mixture of the compound obtained in the previous section (107 mg, 0.233 mmol), THF (2 mL) and 6N HCl_{(g)} (4 mL) was stirred at reflux overnight. The solvent was concentrated and the residue was diluted with EtOAc and washed with saturated aqueous NaHCO₃. The combined organic phases were dried over Na₂SO₄ and concentrated to dryness. The crude product thus obtained was chromatographed over silica gel using hexane/EtOAc mixtures of increasing polarity as eluent, to afford 25 mg of the desired compound (25% yield).

LC-MS (method 2): t_{R}=1.22 min; m/z=405 (MH⁺).

Following a similar procedure to that described in example 8 but using the corresponding starting material, the following compound is obtained:

| Ex- | | Reagent | Reagent | | | |
|---|---|---|---|---|---|---|
| am- | Compound | for | for step | HPLC | t_{R} | |
| ple | name | step a) | b) | method | (min) | m/z |
| 8a | (R)-2-(3- | (R)-methyl | 3- | 4 | 1.42 | 389 |
| | aminosulfonyl- | 3- | amino- | | | |
| | phenyl)amino- | pyrrolidine- | benzene- | | | |
| | 4-(3- | carboxylate | sulfon- | | | |
| | hydroxymethy- | amide | | | | |
| | lpyrrolidin- | | | | | |
| | 1-yl)-7H- | | | | | |
| | pyrrolo[2,3- | | | | | |
| | d]pyrimidine | | | | | |

### Example 9

### 2-(3-Aminosulfonylphenyl)amino-4-[3-(1-hydroxyliminoethyl)piperidin-1-yl]-7H-pyrrolo[2,3-d]pyrimidine

### a) 2-Chloro-4-(3-acetylpiperidin-1-yl)-7H-pyrrolo[2,3-d]pyrimidine

Following a similar procedure to that described in example 1 section a, but using 1-(piperidin-3-yl)ethanone instead of piperidine, the desired product was obtained (39%).

LC-MS (method 2): t_{R}=1.75 min; m/z=279 (MH⁺).

### b) 4-(3-Acetylpiperidin-1-yl)-2-(3-aminosulfonylphenyl)amino-7H-pyrrolo[2,3-d]pyrimidine

Following a similar procedure to that described in example 2 section b, but using 3-aminobenzenesulfonamide, instead of 3-fluoro-4-methoxyphenylamine the product was obtained (37% yield).

LC-MS (method 1): t_{R}=6.26 min; m/z=415 (MH⁺).

### c) Title Compound

To a solution of the compound obtained in previous section (96 mg, 0.233 mmol) in MeOH (3 mL) hydroxylamine hydrochloride (16.2 mg, 0.233 mmol) and sodium acetate (4 mg, 0.023 mmol) were added under Ar-atmosphere. The mixture was stirred at room temperature overnight and the resulting solution was evaporated to dryness, diluted with EtOAc and washed twice with H₂O. The combined organic phases were dried over Na₂SO₄ and concentrated to dryness. The crude product thus obtained was chromatographed over silica gel using hexane/EtOAc mixtures of increasing polarity as eluent, to afford 17 mg of the desired compound (13% yield).

LC-MS (method 1): t_{R}=6.13 min; m/z=430 (MH⁺).

### Example 10

### (S)-2-(3-Aminosulfonylphenyl)amino-4-(2-methoxymethylpyrrolidin-1-yl)-7H-pyrrolo[2,3-d]pyrimidine

### a) (S)-2-Chloro-4-(2-methoxymethylpyrrolidin-1-yl)-7H-pyrrolo[2,3-d]pyrimidine

Following a similar procedure to that described in example 1 section a, but using (S)-2-methoxymethylpyrrolidine, instead of piperidine, and 1,4-dioxane instead of EtOH, 150 mg of the desired product were obtained (83% yield).

### b) (S)-2-(3-Amino-N-tert-butylsulfonylphenyl)amino-4-(2-methoxymethylpyrrolidin-1-yl)-7H-pyrrolo[2,3-d]pyrimidine

Following a similar procedure to that described in example 2 section b, but using 3-amino-N-tert-butylbenzenesulfonamide instead of 3-fluoro-4-methoxyphenylamine, the desired product was obtained (35% yield).

### c) Title Compound

To a solution of the compound obtained in the previous section (0.088 g, 0.19 mmol)) in AcN (2 mL), trifluoromethanesulfonic acid (0.16 mL) was added under Ar-atmosphere and the mixture was stirred at room temperature overnight. The resulting solution was concentrated to dryness, diluted with EtOAc and washed twice with H₂O. The organic phase was dried over Na₂SO₄ and concentrated to dryness. The crude product thus obtained was chromatographed over silica gel using hexane/EtOAc mixtures of increasing polarity as eluent, to obtain 7 mg of the desired compound (22% yield).

LC-MS (method 4): t_{R}=1.77 min; m/z=403 (MH⁺).

### Example 11

### 2-[4-(2-Hydroxyethylaminocarbonyl)phenyl]amino-4-(4-hydroxymethylpiperidin-1-yl)-7H-pyrrolo[2,3-d]pyrimidine

### a) 2-(4-Ethoxycarbonylphenyl)amino-4-(4-hydroxymethylpiperidin-1-yl)-7H-pyrrolo[2,3-d]pyrimidine

Following a similar procedure to that described in example 2 section b, but using the compound obtained in example 2c section a and ethyl 4-aminobenzoate instead of 4-(2-azabicyclo[2.2.1]heptan-2-yl)-2-chloro-7H-pyrrolo[2,3-d]pyrimidine and 3-fluoro-4-methoxyphenylamine, the desired compound was obtained.

### b) 2-(4-Carboxyphenyl)amino-4-(4-hydroxymethylpiperidin-1-yl)-7H-pyrrolo[2,3-d]pyrimidine

To a solution of 354 mg of the compound obtained in the previous section in DME (9 mL), a solution of LiOH.H₂O (188 mg) in 4.5 mL of H₂O was added. The mixture was stirred at room temperature for 40 h. and cooled to 0° C. A 1N aqueous HCl solution (4 mL) was added and the mixture was concentrated. The crude product thus obtained was chromatographed over a SCX-2 column to afford 53 mg of the desired compound.

LC-MS (method 4): t_{R}=1.55 min; m/z=368 (MH⁺).

### c) Title Compound

To a solution of the compound obtained in the previous section (100 mg, 0.2 mmol) in DMF (3 mL), a mixture of EDC.HCl (117 mg, 0.60 mmol), HOBT (82 mg, 0.60 mmol), DIEA (87 μL, 0.60 mmol) and 2-aminoethanol (61 μL, 1.0 mmol) was added under Ar-atmosphere. The resulting mixture was stirred at room temperature overnight and concentrated to dryness. The crude product thus obtained was chromatographed over silica gel using CH₂Cl₂/MeOH/NH₃ mixtures of increasing polarity as eluent, to afford 51 mg of the desired compound (62% yield).

LC-MS (method 4): t_{R}=1.35 min; m/z=411 (MH⁺)

| | | | HPLC | t_{R} | |
|---|---|---|---|---|---|
| Example | Compound name | Starting material | method | (min) | m/z |
| 11a | 4-(4- | 2- | 4 | 1.53 | 425 |
| | hydroxymethylpiperidin- | methoxyethylamine | | | |
| | 1-yl)-2-[4-(2- | | | | |
| | methoxyethylaminocarbonyl)phenyl]amino- | | | | |
| | 7H-pyrrolo[2,3- | | | | |
| | d]pyrimidine | | | | |
| 11b | 4-(4- | 2-(2-aminoethyl)-1- | 4 | 1.25 | 478 |
| | hydroxymethylpiperidin- | methylpyrrolidine | | | |
| | 1-yl)-2-[(4-(2-(1- | | | | |
| | methylpyrrolidin-2- | | | | |
| | yl)ethyl)aminocarbonyl)phenyl]amino- | | | | |
| | 7H- | | | | |
| | pyrrolo[2,3- | | | | |
| | d]pyrimidine | | | | |
| 11c | 4-(4- | 4-(2- | 4 | 1.18 | 480 |
| | hydroxymethylpiperidin- | aminoethyl)morpholine | | | |
| | 1-yl)-2-[(4-(2- | | | | |
| | (morpholin-4- | | | | |
| | yl)ethyl)aminocarbonyl)phenyl]amino- | | | | |
| | 7H- | | | | |
| | pyrrolo[2,3- | | | | |
| | d]pyrimidine | | | | |

### Example 12

### (S)-2-(3-Aminosulfonylphenyl)amino-4-(3-(2-hydroxypropan-2-yl)piperidin-1-yl)-7H-pyrrolo[2,3-d]pyrimidine

### a) (S)-2-Chloro-4-(3-ethoxycarbonylpiperidin-1-yl)-7H-pyrrolo[2,3-d]pyrimidine

Following a similar procedure to that described in example 1 section a, but using (S)-ethyl 3-piperidinecarboxylate instead of piperidine, the desired compound was obtained.

LC-MS (method 4): t_{R}=3.01 min; m/z=309 (MH⁺).

### b) (S)-2-(3-Aminosulfonylphenyl)amino-4-(3-ethoxycarbonylpiperidin-1-yl)-7H-pyrrolo[2,3-d]pyrimidine

Following a similar procedure to that described in example 2 section b, but using the compound obtained in the previous section and 3-aminobenzenesulfonamide instead of 4-(2-azabicyclo[2.2.1]heptan-2-yl)-2-chloro-7H-pyrrolo[2,3-d]pyrimidine and 3-fluoro-4-methoxyphenylamine, the desired compound was obtained.

LC-MS (method 4): t_{R}=2.05 min; m/z=445 (MH⁺).

### c) Title Compound

To a solution of the compound obtained in the previous section (65 mg, 0.15 mmol) in THF (3 mL), a 1.4 M solution of methylmagnesium bromide in THF (0.75 mL, 1.05 mmol) was added at 0° C. The resulting mixture was stirred under Ar-atmosphere at room temperature overnight. The mixture was concentrated to dryness and the residue thus obtained was chromatographed over silica gel using CH₂Cl₂/MeOH/NH₃ mixtures of increasing polarity as eluent, to afford 17 mg of the desired compound (26% yield).

LC-MS (method 4): t_{R}=1.78 min; m/z=431 (MH⁺)

### Example 13

### 2-(3-Aminosulfonylphenyl)amino-4-(7-oxo-6-azabicyclo[3.2.1]octan-6-yl)-7H-pyrrolo[2,3-d]pyrimidine

### a) 4-(3-Carboxycyclohexylamino)-2-chloro-7H-pyrrolo[2,3-d]pyrimidine

To a solution of 2,4-dichloro-7H-pyrrolo[2,3-d]pyrimidine (0.50 g, 2.66 mmol) in THF/H₂O (1:1) (7 mL), 3-aminocyclohexane carboxylic acid (0.38 g, 2.66 mmol) and K₂CO₃ (0.55 g, 3.98 mmol) were added. The reaction was stirred at 110° C. in a sealed tube for 10 h. The resulting mixture was diluted with H₂O and the phases were separated. Aqueous 1N HCl was added at 0° C. until pH=3 and extracted thrice with EtOAc/MeOH (9:1). The combined organic phases were dried over Na₂SO₄ and concentrated to dryness and the crude product thus obtained was directly used in the next step.

LC-MS (method 2): t_{R}=0.94 min; m/z=295 (MH⁺).

### a) 2-Chloro-4-(7-oxo-6-azabicyclo[3.2.1]octan-6-yl)-7H-pyrrolo[2,3-d]pyrimidine

To a solution of the product obtained in the previous section in DMF (25 mL), HBTU (1.14 g, 3.00 mmol) and DIEA (0.65 mL, 3.73 mmol) were added. The reaction was stirred under Ar-atmosphere at room temperature for 18 h. The resulting mixture was concentrated to dryness and the residue was dissolved in DMF (20 mL). DIEA (0.65 mL, 3.73 mmol) was added and the mixture was stirred overnight at 120° C. The resulting mixture was evaporated to dryness and the crude product thus obtained was chromatographed over silica gel using CHCl₃/MeOH mixtures of increasing polarity as eluent, to afford 0.15 g of the desired compound (20% yield).

LC-MS (method 2): t_{R}=1.95 min; m/z=277 (MH⁺).

### b) Title Compound

Following a similar procedure to that described in example 2 section b, but using the compound obtained in the previous section, and 3-aminobenzenesulfonamide instead of 4-(2-azabicyclo[2.2.1]heptan-2-yl)-2-chloro-7H-pyrrolo[2,3-d]pyrimidine and 3-fluoro-4-methoxyphenylamine, the desired product was obtained (19% yield).

LC-MS (method 2): t_{R}=1.72 min; m/z=413 (MH⁺).

### Example 14

### Biological Assay 1

### JAK3 Kinase Inhibition

In a final volume of 50 μL, 5 μL of the test product dissolved in 10% DMSO (final concentration, 0.001-10 μM), was incubated with 4 μg/mL of human JAK3 781-1124, 1 μg/mL of Poly-L-Ala, L-Glu, L-Lys, L-Tyr and ATP (0.2 μM, approximately 2×10⁵ cpm of γ³³P-ATP) in HEPES buffer (60 mM, pH 7.5) with Mg²⁺chloride (3 mM), Mn²⁺chloride (3 mM), sodium orthovanadate (3 μM) and dithiothreitol (1.2 mM). The reaction was started by adding Mg²⁺[γ³³P-ATP]. After incubation for 50 min at room temperature, the reaction was quenched by the addition of 50 μL of 2% phosphoric acid solution. The reaction mixture was filtered in vacuo and washed three times with a 150 mM phosphoric acid solution. 200 μL of liquid scintillation was added before drying it and counting it.

The compounds of all examples showed more than 50% of inhibition of JAK3 activity at 10 μM in this assay.

1-40. (canceled)

41. A compound of Formula (I): 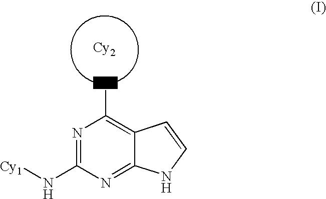
or a salt thereof, wherein:
Cy₁ is phenyl or a 5- to 6-membered aromatic heterocycle bonded to the NH group through a C atom, each of which is optionally fused to a 5- or 6-membered saturated, partially unsaturated or aromatic carbocyclic or heterocyclic ring, wherein Cy₁ optionally comprises from 1 to 4 heteroatoms selected from N, O and S, wherein one or more C or S atoms of the optional 5- or 6-membered fused ring is optionally replaced with one or more groups selected from CO, SO and SO₂ groups, and wherein Cy₁ is optionally substituted with one or more R₁;
Cy₂ is a 3- to 7-membered monocyclic or 6- to 11-membered bicyclic heterocycle, wherein the ring which contains the N atom bonded to the pyrrolopyrimidine moiety is saturated or partially unsaturated, wherein Cy₂ comprises from 1 to 4 heteroatoms selected from N, O and S, wherein one or more C or S atoms of said monocyclic or bicyclic is optionally replaced with one or more groups selected from CO, SO and SO₂, and wherein Cy₂ is optionally substituted with one or more R₂;
each R₁ is independently selected from C₁₋₄ alkyl, C₂₋₄ alkenyl, C₂₋₄ alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —C(═N—OH)R₄ or Cy₃, wherein C₁₋₄ alkyl, C₂₋₄ alkenyl and C₂₋₄ alkynyl is optionally substituted with one or more R₆, and wherein Cy₃ is optionally substituted with one or more R₇;
each R₂ is independently selected from C₁₋₄ alkyl, C₂₋₄ alkenyl, C₂₋₄ alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —NR₅SO₂R₄, —C(═N—OH)R₄ or Cy₃, wherein C₁₋₄ alkyl, C₂₋₄ alkenyl and C₂₋₄ alkynyl is optionally substituted with one or more R₆, and wherein Cy₃ is optionally substituted with one or more R₇;
R₃ is hydrogen or R₄;
R₄ is C₁₋₄ alkyl, C₂₋₄ alkenyl, C₂₋₄ alkynyl, or Cy₄, wherein C₁₋₄ alkyl, C₂₋₄ alkenyl and C₂₋₄ alkynyl are optionally substituted with one or more R₆, and wherein Cy₄ is optionally substituted with one or more R₈;
R₅ is hydrogen or C₁₋₄ alkyl;
R₆ is halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, —C(═N—OH)R₁₀ or Cy₄, wherein Cy₄ is optionally substituted with one or more R₈;
R₇ is a C₁₋₄ alkyl optionally substituted with one or more R₁₁, or R₇ is selected one or more R₁₂;
R₈ is C₁₋₄ alkyl, haloC₁₋₄ alkyl, C₁₋₄ alkoxyC₁₋₄ alkyl, hydroxyC₁₋₄ alkyl, cyanoC₁₋₄alkyl, or R₈ is selected from one or more R₁₂;
R₉ is hydrogen or R₁₀;
R₁₀ is C₁₋₄ alkyl, haloC₁₋₄ alkyl, C₁₋₄ alkoxyC₁₋₄ alkyl, hydroxyC₁₋₄ alkyl, cyanoC₁₋₄alkyl, Cy₅-C₁₋₄ alkyl or Cy₄, wherein Cy₄ is optionally substituted with one or more R₈;
R₁₁ is halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, or —C(═N—OH)R₁₀;
R₁₂ is halogen, —CN, —NO₂, —COR₁₃, —CO₂R₁₃, —CONR₁₃R₁₃, —OR₁₃, —OCOR₁₄, —OCONR₁₄R₁₄, —OCO₂R₁₄, —SR₁₃, —SOR₁₄, —SO₂R₁₄, —SO₂NR₁₃R₁₃, —SO₂NR₅COR₁₄, —NR₁₃R₁₃, —NR₅COR₁₃, —NR₅CONR₁₃R₁₃, —NR₅CO₂R₁₄, —NR₅SO₂R₁₄ or —C(═N—OH)R₁₄;
R₁₃ is hydrogen or R₁₄;
R₁₄ is C₁₋₄ alkyl, haloC₁₋₄ alkyl, C₁₋₄ alkoxyC₁₋₄ alkyl or hydroxyC₁₋₄ alkyl;
or two R₁₃ groups or two R₁₄ groups on the nitrogen atom to which they are attached, form a 5- or 6-membered saturated ring, optionally comprising one or two heteroatoms selected from N, S and O, wherein said heteroatoms are optionally substituted with one or more C₁₋₄ alkyl groups;
Cy₃ and Cy₄ are each independently selected from a 3- to 7-membered monocyclic or 6- to 11-membered bicyclic ring, wherein said monocyclic and bicyclic rings are optionally carbocyclic or heterocyclic, wherein the heterocyclic ring system optionally comprises from 1 to 4 heteroatoms selected from N, S and O, wherein each Cy₃ and Cy₄ is optionally saturated, partially unsaturated or aromatic, and is optionally bonded to the rest of the molecule through any available C or N atom, and wherein one or more C or S atoms of the ring is optionally replaced by one or more groups selected from CO, SO and SO₂ groups;
Cy₅ is selected from (a), (b), or (c): 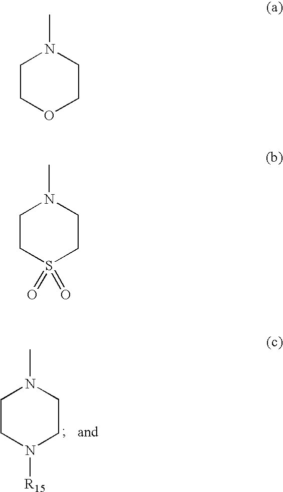
R₁₅ is hydrogen or C₁₋₄ alkyl.

42. A compound according to claim 41, wherein Cy₁ is phenyl substituted with one or more R₁.

43. A compound according to claim 42, wherein Cy₁ is phenyl substituted at one or two of positions 3, 4 and 5 with R₁.

44. A compound according to claim 43, wherein Cy₁ is phenyl substituted at position 3 or 4 with R₁.

45. A compound according to claim 41, wherein Cy₁ is a 5- or 6-membered aromatic heterocycle bonded to the NH group through a C atom, each of which is optionally fused to a 5- or 6-membered saturated, partially unsaturated or aromatic carbocyclic or heterocyclic ring, wherein Cy₁ optionally comprises from 1 to 4 heteroatoms selected from N, O and S, wherein one or more C or S atoms of the optional 5- or 6-membered fused ring is optionally replaced with one or more groups selected from CO, SO and SO₂ groups, and wherein Cy₁ is optionally substituted with one or more R₁;

46. A compound according to claim 41, wherein R₁ is C₁₋₄ alkyl, halogen, —CN, —COR₃, —CO₂R₃, —CONR₃R₃, —COCONR₃R₃, —OR₃, —SR₃, —SO₂R₄, —SO₂NR₃R₃, —SO₂NR₅COR₄, —NR₃R₃, —NR₅COR₃, —NR₅CONR₃R₃, or Cy₃, wherein the C₁₋₄ alkyl group is optionally substituted with one or more R₆, and wherein Cy₃ is optionally substituted with one or more R₇.

47. A compound according to claim 46, wherein R₁ is C₁₋₄ alkyl, halogen, —CONR₃R₃, —OR₃, —SO₂NR₃R₃, —SO₂NR₅COR₄, —NR₅COR₃ or Cy₃, wherein the C₁₋₄ alkyl group is optionally substituted with one or more R₆, and wherein Cy₃ is optionally substituted with one or more R₇.

48. A compound according to claim 47, wherein R₁ is C₁₋₄ alkyl, hydroxyC₁₋₄ alkyl, C₁₋₄ alkoxyC₁₋₄ alkyl, Cy₄-C₁₋₄ alkyl, NR₉R₉SO₂—C₁₋₄ alkyl, NR₉R₉CO—C₁₋₄ alkyl, R₁₀CONR₅SO₂—C₁₋₄ alkyl, R₉CONR₅—C₁₋₄ alkyl, halogen, —CONR₃R₃, —OR₃, —SO₂NR₃R₃, —SO₂NR₅COR₄, —NR₅COR₃ or Cy₃, wherein Cy₃ is optionally substituted with one or more R₇ and wherein Cy₄ is optionally substituted with one or more R₈.

49. A compound according to claim 41, wherein
R₁ is Cy₃; and
Cy₃ is a 5- or 6-membered saturated monocyclic heterocycle comprising 1 or 2 heteroatoms selected from N, S and O, wherein said heterocycle is optionally bonded to the rest of the molecule through any available C or N atom, and wherein one or more C or S atoms of the ring is optionally replaced with one or more groups selected from CO, SO and SO₂, and wherein said Cy₃ is optionally substituted with one or more R₇.

50. A compound according to claim 49, wherein said 5- or 6-membered saturated monocyclic heterocycle comprises at least one N heteroatom.

51. A compound according to claim 41, wherein Cy₄ is a 5- or 6-membered saturated monocyclic heterocycle comprising 1 or 2 heteroatoms selected from N, S and O, wherein said heterocycle is optionally bonded to the rest of the molecule through any available C or N atom, wherein one or more C or S of the ring atoms is optionally replaced with one or more groups selected from CO, SO and SO₂ groups and wherein said Cy₄ₐ is optionally substituted with one or more R₈.

52. A compound according to claim 41, wherein for R₁,
R₃ is hydrogen or R₄; and
R₄ is C₁₋₄ alkyl or Cy₄, wherein said C₁₋₄ alkyl is optionally substituted with one or more R₆, and wherein Cy₄ is optionally substituted with one or more R₈.

53. A compound according to claim 52, wherein
R₃ is hydrogen or R₄; and
R₄ is C₁₋₄ alkyl, Cy₄-C₁₋₄ alkyl, hydroxyC₁₋₄ alkyl, C₁₋₄ alkoxy C₁₋₄ alkyl or Cy₄, wherein any of Cy₄ is optionally substituted with one or more R₈.

54. A compound according to claim 41, wherein Cy₂ is selected from a saturated 5- to 7-membered monocyclic or 6- to 11-membered bicyclic heterocycle, and wherein Cy₂ comprises 1 to 3 heteroatoms selected from N, O and S, and wherein one or more of the ring atoms are optionally replaced with one or more groups selected from CO, SO and SO₂, and wherein Cy₂ is optionally substituted with one or more R₂.

55. A compound according to claim 54, wherein Cy₂ is selected from (a) to (i): 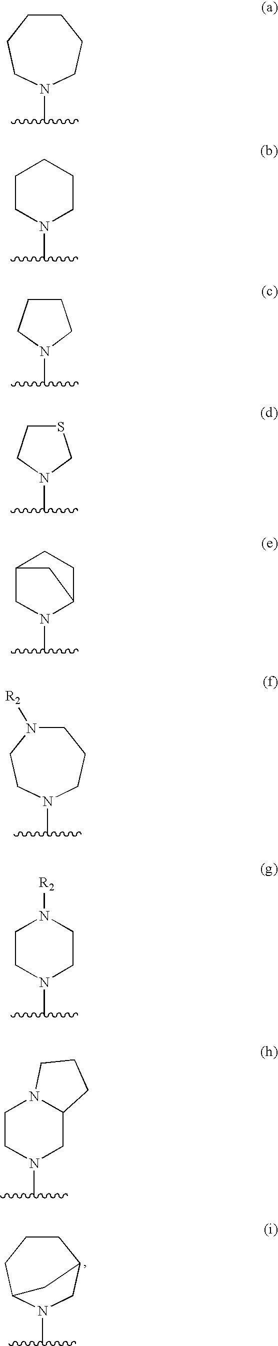
wherein one or more C or S atoms of Cy₂ is optionally replaced with one or more groups selected from CO, SO and SO₂ groups, and wherein Cy₂ is optionally substituted with one or more R₂.

56. A compound according to claim 55, wherein Cy₂ is (b): 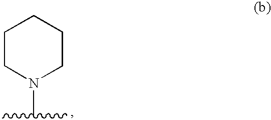
wherein one or more C atoms of Cy₂ is optionally replaced with one or more CO groups, and wherein Cy₂ is optionally substituted with one or more R₂.

57. A compound according to claim 55, wherein Cy₂ is (c): 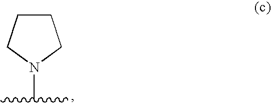
wherein one or more C atoms of Cy₂ is optionally replaced with one or more CO groups, and wherein Cy₂ is optionally substituted with one or more R₂.

58. A compound according to claim 41, wherein each R₂ is independently selected from C₁₋₄ alkyl, halogen, —CN, —COR₃, —CO₂R₃, —CONR₃R₃, —OR₃, —NR₃R₃, —NR₅COR₃, —NR₅CONR₃R₃, —NR₅SO₂R₄ or Cy₃, wherein C₁₋₄ alkyl is optionally substituted with one or more R₆ and wherein Cy₃ is optionally substituted with one or more R₇.

59. A compound according to claim 58, wherein each R₂ is independently selected from C₁₋₄ alkyl, C₁₋₄ alkoxyC₁₋₄ alkyl, hydroxyC₁₋₄ alkyl, haloC₁₋₄alkyl, halogen, —CN, —COR₃, —CO₂R₃, —CONR₃R₃, —OR₃, —NR₃R₃, —NR₅COR₃ or Cy₃, wherein Cy₃ is optionally substituted with one or more R₇.

60. A compound according to claim 59, wherein each R₂ is independently selected from C₁₋₄ alkyl, —COR₃, —OR₃, —NR₃R₃, —NR₅COR₃, —NR₅CONR₃R₃ or —NR₅SO₂R₄, wherein C₁₋₄ alkyl is optionally substituted with one or more R₆.

61. A compound according to claim 60, wherein each R₂ is independently selected from C₁₋₄ alkyl, C₁₋₄ alkoxyC₁₋₄ alkyl, hydroxyC₁₋₄ alkyl, haloC₁₋₄ alkyl, Cy₄-C₁₋₄ alkyl, R₉CO—C₁₋₄ alkyl, NR₉R₉—C₁₋₄alkyl, R₉CONR₅—C₁₋₄ alkyl, R₁₀SO₂NR₅—C₁₋₄alkyl, NR₉R₉CO—C₁₋₄ alkyl, NR₉R₉CONR₅—C₁₋₄ alkyl, —COR₃, —OR₃, —NR₃R₃, —NR₅COR₃, —NR₅CONR₃R₃ or —NR₅SO₂R₄, wherein Cy₄ is optionally substituted with one or more R₈.

62. A compound according to claim 41, wherein
R₂ is Cy₃; and
Cy₃ is a saturated 3- to 7-membered monocyclic or 6- to 11-membered bicyclic ring wherein said monocyclic and bicyclic ring is optionally carbocyclic or heterocyclic, wherein the heterocyclic ring system optionally comprises from 1 to 4 heteroatoms selected from N, S and O, wherein Cy₃ is optionally bonded to the rest of the molecule through any available C or N atom, wherein one or more C or S atoms of the ring is optionally replaced with one or more groups selected from CO, SO and SO₂ groups, and wherein Cy₃ is optionally substituted with one or more R₇.

63. A compound according to claim, 41 wherein for R₂,
R₃ is hydrogen or R₄; and
R₄ is C₁₋₄ alkyl optionally substituted with one or more R₆.

64. A pharmaceutical composition comprising a compound according to claim 41, or a pharmaceutically acceptable salt thereof, and one or more pharmaceutically acceptable excipients.

65. A method for the treatment at least one disease selected from transplant rejection, immune, autoimmune and inflammatory diseases, neurodegenerative diseases, and proliferative disorders, in a subject in need thereof, comprising administering to said subject an effective amount of at least one compound of Formula (I): 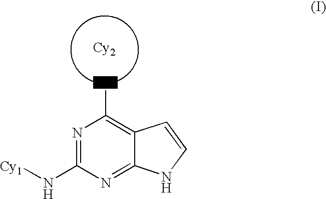
or a pharmaceutically acceptable salt thereof, wherein:
Cy₁ is phenyl or a 5- to 6-membered aromatic heterocycle bonded to the NH group through a C atom, each of which is optionally fused to a 5- or 6-membered saturated, partially unsaturated or aromatic carbocyclic or heterocyclic ring, wherein Cy₁ optionally comprises from 1 to 4 heteroatoms selected from N, O and S, wherein one or more C or S atoms of the optional 5- or 6-membered fused ring is optionally replaced with one or more groups selected from CO, SO and SO₂ groups, and wherein Cy₁ is optionally substituted with one or more R₁;
Cy₂ is a 3- to 7-membered monocyclic or 6- to 11-membered bicyclic heterocycle, wherein the ring which contains the N atom bonded to the pyrrolopyrimidine moiety is saturated or partially unsaturated, wherein Cy₂ comprises from 1 to 4 heteroatoms selected from N, O and S, wherein one or more C or S atoms of said monocyclic or bicyclic is optionally replaced with one or more groups selected from CO, SO and SO₂, and wherein Cy₂ is optionally substituted with one or more groups selected from R₂;
each R₁ is independently selected from C₁₋₄ alkyl, C₂₋₄ alkenyl, C₂₋₄ alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —C(═N—OH)R₄ or Cy₃, wherein C₁₋₄ alkyl, C₂₋₄ alkenyl and C₂₋₄ alkynyl is optionally substituted with one or more R₆, and wherein Cy₃ is optionally substituted with one or more R₇;
each R₂ is independently selected from C₁₋₄ alkyl, C₂₋₄ alkenyl, C₂₋₄ alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —NR₅SO₂R₄, —C(═N—OH)R₄ or Cy₃, wherein C₁₋₄ alkyl, C₂₋₄ alkenyl and C₂₋₄ alkynyl is optionally substituted with one or more R₆, and wherein Cy₃ is optionally substituted with one or more R₇;
R₃ is hydrogen or R₄;
R₄ is C₁₋₄ alkyl, C₂₋₄ alkenyl, C₂₋₄ alkynyl, or Cy₄, wherein C₁₋₄ alkyl, C₂₋₄ alkenyl and C₂₋₄ alkynyl is optionally substituted with one or more R₆, and wherein Cy₄ is optionally substituted with one or more R₈;
R₅ is hydrogen or C₁₋₄ alkyl;
R₆ is halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, —C(═N—OH)R₁₀ or Cy₄, wherein Cy₄ is optionally substituted with one or more R₈;
R₇ is a C₁₋₄ alkyl optionally substituted with one or more R₁₁, or R₇ is selected one or more R₁₂;
R₈ is C₁₋₄ alkyl, haloC₁₋₄ alkyl, C₁₋₄ alkoxyC₁₋₄ alkyl, hydroxyC₁₋₄ alkyl, cyanoC₁₋₄ alkyl, or R₈ is selected from one or more R₁₂;
R₉ is hydrogen or R₁₀;
R₁₀ is C₁₋₄ alkyl, haloC₁₋₄ alkyl, C₁₋₄ alkoxyC₁₋₄ alkyl, hydroxyC₁₋₄ alkyl, cyanoC₁₋₄ alkyl, Cy₅-C₁₋₄ alkyl or Cy₄, wherein Cy₄ is optionally substituted with one or more R₈;
R₁₁ is halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, or —C(═N—OH)R₁₀;
R₁₂ is halogen, —CN, —NO₂, —COR₁₃, —CO₂R₁₃, —CONR₁₃R₁₃, —OR₁₃, —OCOR₁₄, —OCONR₁₄R₁₄, —OCO₂R₁₄, —SR₁₃, —SOR₁₄, —SO₂R₁₄, —SO₂NR₁₃R₁₃, —SO₂NR₅COR₁₄, —NR₁₃R₁₃, —NR₅COR₁₃, —NR₅CONR₁₃R₁₃, —NR₅CO₂R₁₄, —NR₅SO₂R₁₄ or —C(═N—OH)R₁₄;
R₁₃ is hydrogen or R₁₄;
R₁₄ is C₁₋₄ alkyl, haloC₁₋₄ alkyl, C₁₋₄ alkoxyC₁₋₄ alkyl or hydroxyC₁₋₄ alkyl;
or two R₁₃ groups or two R₁₄ groups on the nitrogen atom to which they are attached, form a 5- or 6-membered saturated ring, optionally comprising one or two heteroatoms selected from N, S and O, wherein said heteroatoms are optionally substituted with one or more C₁₋₄ alkyl groups;
Cy₃ and Cy₄ are each independently selected from a 3- to 7-membered monocyclic or 6- to 11-membered bicyclic ring, wherein said monocyclic and bicyclic rings are optionally carbocyclic or heterocyclic, wherein the heterocyclic ring system optionally comprises from 1 to 4 heteroatoms selected from N, S and O, wherein each Cy₃ and Cy₄ is optionally saturated, partially unsaturated or aromatic, and is optionally bonded to the rest of the molecule through any available C or N atom, and wherein one or more C or S atoms of the ring is optionally replaced by one or more groups selected from CO, SO and SO₂ groups;
Cy₅ is selected from (a), (b), or (c): 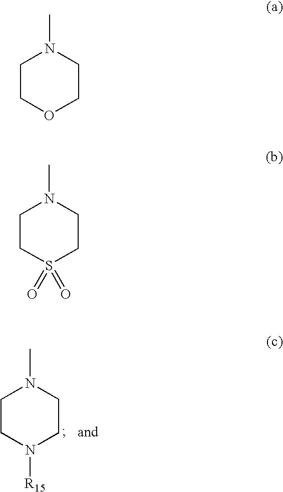
R₁₅ is hydrogen or C₁₋₄ alkyl.

66. The method according to claim 65, wherein the disease is selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas, and thromboembolic and allergic complications associated with leukemias and lymphomas.

67. A process for the preparation of a compound of Formula (I) 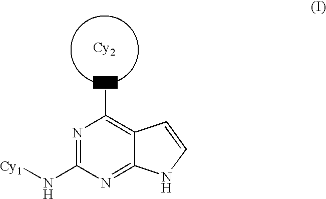
or a salt thereof, wherein:
Cy₁ is phenyl or a 5- to 6-membered aromatic heterocycle bonded to the NH group through a C atom, each of which is optionally fused to a 5- or 6-membered saturated, partially unsaturated or aromatic carbocyclic or heterocyclic ring, wherein Cy₁ optionally comprises from 1 to 4 heteroatoms selected from N, O and S, wherein one or more C or S atoms of the optional 5- or 6-membered fused ring is optionally replaced with one or more groups selected from CO, SO and SO₂ groups, and wherein Cy₁ is optionally substituted with one or more R₁;
Cy₂ is a 3- to 7-membered monocyclic or 6- to 11-membered bicyclic heterocycle, wherein the ring which contains the N atom bonded to the pyrrolopyrimidine moiety is saturated or partially unsaturated, wherein Cy₂ comprises from 1 to 4 heteroatoms selected from N, O and S, wherein one or more C or S atoms of said monocyclic or bicyclic is optionally replaced with one or more groups selected from CO, SO and SO₂, and wherein Cy₂ is optionally substituted with one or more groups selected from R₂;
each R₁ is independently selected from C₁₋₄ alkyl, C₂₋₄ alkenyl, C₂₋₄ alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —C(═N—OH)R₄ or Cy₃, wherein C₁₋₄ alkyl, C₂₋₄ alkenyl and C₂₋₄ alkynyl is optionally substituted with one or more R₆, and wherein Cy₃ is optionally substituted with one or more R₇;
each R₂ is independently selected from C₁₋₄ alkyl, C₂₋₄ alkenyl, C₂₋₄ alkynyl, halogen, —CN, —NO₂, —COR₃, —CO₂R₃, —CONR₃R₃, —COCONR₃R₃, —OR₃, —OCOR₄, —OCONR₄R₄, —OCO₂R₄, —SR₃, —SOR₄, —SO₂R₄, —SO₂NR₃R₃, —SO₂NR₅COR₄, —NR₃R₃, —NR₅COR₃, —NR₅CONR₃R₃, —NR₅CO₂R₄, —NR₅SO₂R₄, —C(═N—OH)R₄ or Cy₃, wherein C₁₋₄ alkyl, C₂₋₄ alkenyl and C₂₋₄ alkynyl is optionally substituted with one or more R₆, and wherein Cy₃ is optionally substituted with one or more R₇;
R₃ is hydrogen or R₄;
R₄ is C₁₋₄ alkyl, C₂₋₄ alkenyl, C₂₋₄ alkynyl, or Cy₄, wherein C₁₋₄ alkyl, C₂₋₄ alkenyl and C₂₋₄ alkynyl are optionally substituted with one or more R₆, and wherein Cy₄ is optionally substituted with one or more R₈;
R₅ is hydrogen or C₁₋₄ alkyl;
R₆ is halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, —C(═N—OH)R₁₀ or Cy₄, wherein Cy₄ is optionally substituted with one or more R₈;
R₇ is a C₁₋₄ alkyl optionally substituted with one or more R₁₁, or R₇ is selected from one or more R₁₂;
R₈ is C₁₋₄ alkyl, haloC₁₋₄ alkyl, C₁₋₄ alkoxyC₁₋₄ alkyl, hydroxyC₁₋₄ alkyl, cyanoC₁₋₄ alkyl, or R₈ is selected from one or more R₁₂;
R₉ is hydrogen or R₁₀;
R₁₀ is C₁₋₄ alkyl, haloC₁₋₄ alkyl, C₁₋₄ alkoxyC₁₋₄ alkyl, hydroxyC₁₋₄ alkyl, cyanoC₁₋₄ alkyl, Cy₅-C₁₋₄ alkyl or Cy₄, wherein Cy₄ is optionally substituted with one or more R₈;
R₁₁ is halogen, —CN, —NO₂, —COR₉, —CO₂R₉, —CONR₉R₉, —OR₉, —OCOR₁₀, —OCONR₁₀R₁₀, —OCO₂R₁₀, —SR₉, —SOR₁₀, —SO₂R₁₀, —SO₂NR₉R₉, —SO₂NR₅COR₁₀, —NR₉R₉, —NR₅COR₉, —NR₅CONR₉R₉, —NR₅CO₂R₁₀, —NR₅SO₂R₁₀, or —C(═N—OH)R₁₀;
R₁₂ is halogen, —CN, —NO₂, —COR₁₃, —CO₂R₁₃, —CONR₁₃R₁₃, —OR₁₃, —OCOR₁₄, —OCONR₁₄R₁₄, —OCO₂R₁₄, —SR₁₃, —SOR₁₄, —SO₂R₁₄, —SO₂NR₁₃R₁₃, —SO₂NR₅COR₁₄, —NR₁₃R₁₃, —NR₅COR₁₃, —NR₅CONR₁₃R₁₃, —NR₅CO₂R₁₄, —NR₅SO₂R₁₄ or —C(═N—OH)R₁₄;
R₁₃ is hydrogen or R₁₄;
R₁₄ is C₁₋₄ alkyl, haloC₁₋₄ alkyl, C₁₋₄ alkoxyC₁₋₄ alkyl or hydroxyC₁₋₄ alkyl;
or two R₁₃ groups or two R₁₄ groups on the nitrogen atom to which they are attached, form a 5- or 6-membered saturated ring, optionally comprising one or two heteroatoms selected from N, S and O, wherein said heteroatoms are optionally substituted with one or more C₁₋₄ alkyl groups;
Cy₃ and Cy₄ are each independently selected from a 3- to 7-membered monocyclic or 6- to 11-membered bicyclic ring, wherein said monocyclic and bicyclic rings are optionally carbocyclic or heterocyclic, wherein the heterocyclic ring system optionally comprises from 1 to 4 heteroatoms selected from N, S and O, wherein each Cy₃ and Cy₄ is optionally saturated, partially unsaturated or aromatic, and is optionally bonded to the rest of the molecule through any available C or N atom, and wherein one or more C or S atoms of the ring is optionally replaced by one or more groups selected from CO, SO or SO₂ groups;
Cy₅ is selected from (a), (b), or (c): 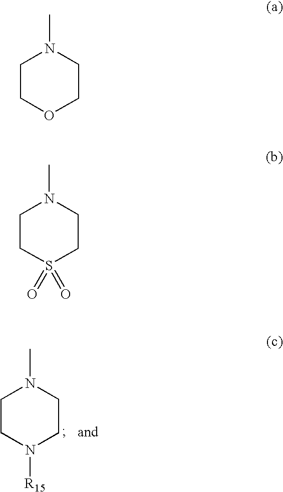
R₁₅ is hydrogen or C₁₋₄ alkyl,
the process comprising:
(a) reacting a compound of Formula (IV) with a compound of Formula (V) 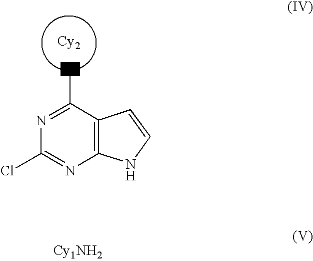
wherein Cy₂ and Cy₂ are as described above; or
(b) converting, in one or a plurality of steps, a compound of Formula (I) into another compound of Formula (I).
